(19) United States
(12) Patent Application Publication
(10) Pub. No.: US 2023/0192644 A1
(43) Pub. Date: Jun. 22, 2023

(54) PIPERIDINE-2,6-DIONES AS SMALL MOLECULE DEGRADERS OF HELIOS AND METHODS OF USE

(71) Applicant: DANA-FARBER CANCER INSTITUTE, INC., Boston, MA (US)

(72) Inventors: Nathanael S. Gray, Stanford, CA (US); Eric S. Fischer, Chestnut Hill, MA (US); Alyssa Verano, Allston, MA (US); Hu Liu, Newton, MA (US); Tinghu Zhang, Brookline, MA (US); Lyn H. Jones, Winchester, MA (US); Eric S. Wang, Brookline, MA (US); Radoslaw Nowak, Boston, MA (US); Jianwei Che, Sharon, MA (US)

(73) Assignee: DANA-FARBER CANCER INSTITUTE, INC., Boston, MA (US)

(21) Appl. No.: 17/926,074

(22) PCT Filed: May 20, 2021
(86) PCT No.: PCT/US2021/033328

§ 371 Date: Nov. 17, 2022

Related U.S. Application Data
(60) Provisional application No. 63/028,011, filed on May 21, 2020.

(51) Int. Cl. C07D 401/04; C07D 401/14; C07D 405/14

(52) U.S. Cl. CPC C07D 401/04; C07D 401/14; C07D 405/14

(57) ABSTRACT

Disclosed are compounds and pharmaceutically acceptable salts, hydrates, solvates, prodrugs, stereoisomers, and tautomers thereof that may cause degradation of various proteins e.g., IKZF2 (Helios). Also disclosed are pharmaceutical compositions containing same, and methods of making and using the compounds to treat diseases and disorders associated with Helios and which may benefit from Helios degradation.

### RELATED APPLICATIONS

This application claims the benefit of priority under 35 U.S.C. § 119(e) to U.S. Provisional Application No. 63/028,011, filed May 21, 2020, which is incorporated herein by reference in its entirety.

### GOVERNMENT LICENSE RIGHTS

This invention was made with government support under grant number R01 CA214608-03 awarded by the National Institutes of Health. The government has certain rights in the invention.

### BACKGROUND OF THE INVENTION

Imide molecules, such as thalidomide and its analogs, bind to Cereblon (CRBN), a substrate adaptor for the ubiquitously expressed cullin ring ligase 4 (CUL4)-RBX1-DDB1-CRBN (CUL4CRBN) E3 ligase (Kronke et al., Science 343:301-305 (2014); Ito et al., Science 327:1345-1350 (2010)). This results in the recruitment, ubiquitination, and the subsequent proteasomal degradation of neo-substrates, namely Ikaros (IKZF1) and Aiolos (IKZF3), but not any other members of the IKZF zinc finger transcription factor family. CC-885, an imide analog, is predicted to have some activity in inducing Helios degradation, but also induces degradation of GSPT1, a key translation termination factor (Matyskiela et al., Nature 535:252-257 (2016)).

Helios (IKZF2), a member of the IKZF family, is a critical regulator of T cell activity and function. Genetic deletion of Helios resulted in an enhanced anti-tumor immune response (Kim et al., Science 350:334-339 (2015)). Notably, Helios is highly expressed in regulatory T cells (Elkord et al., Expert Opin. Biol. Ther. 12:1423-1425 (2012)), a subpopulation of T cells that restricts the activity of effector T cells. Selective deletion of Helios in regulatory T cells resulted in both loss of suppressive activity and acquisition of effector T cell functions (Najagawa et al., Proc. Natl. Acad. Sci. USA 113:6248-6253 (2016); Yates et al., Proc. Natl. Acad. Sci. USA 115:2162-2167 (2018)). Thus, Helios is a critical factor in restricting T cell effector function in Tregs.

Helios expression has also been reported to be upregulated in ‘exhausted’ T cells, in the settings of both chronic viral infections (Crawford et al., Immunity 40:289-302 (2014), Doering et al., Immunity 371130-1144 (2012); Scott-Browne et al., Immunity 45:1327-1340 (2016)) and tumors (Martinez et al., Immunity 42:265-278 (2015); Mognol et al., Proc. Natl. Acad. Sci. USA 114:E2776-E2785 (2017); Pereira et al., J. Leukoc. Biol. 102:601-615 (2017); Singer et al., Cell 166:1500-1511 (2016); Schietinger et al., Immunity 45:389-401 (2016)), as well as in dysfunctional chimeric antigen receptor (CAR) T cells (Long et al., Nat. Med. 21:581-590 (2015)). Overexpression or aberrant expression of Helios and various splice isoforms have been reported in several hematological malignancies, including T cell leukemias and lymphomas (Nakase at al., Exp. Hematol. 30:313-317 (2002); Tabayashi et al., Cancer Sci. 98:182-188 (2007); Asanuma et al., Cancer Sci. 104:1097-1106 (2013)). Moreover, knockdown of Helios in a model of mixed lineage leukemia (MLL)-driven myeloid leukemia potently suppressed proliferation and increased cell death (Park et al., J. Clin. Invest. 125:1286-1298 (2015); Park et al., Cell Stem Cell 24:153-165 (2019)).

### SUMMARY OF THE INVENTION

A first aspect of the present invention is directed to a compound represented by a structure of formula (I):

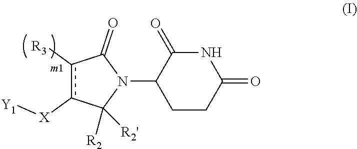

wherein R₂, R₂′, R₃, m₁, X, and Y₁ are as defined herein, or a pharmaceutically acceptable salt, hydrate, solvate, prodrug, stereoisomer, or tautomer thereof.

Another aspect of the present invention is directed to a pharmaceutical composition that includes a therapeutically effective amount of a compound of formula (I) or a pharmaceutically acceptable salt, hydrate, solvate, prodrug, stereoisomer, or tautomer thereof, and a pharmaceutically acceptable carrier. In some embodiments, the pharmaceutical composition comprises a co-crystal.

Another aspect of the present invention is directed to methods of treating diseases or disorders characterized or mediated by activity of a protein that is a substrate for a complex between cereblon (CRBN) and an inventive compound, that entail the administration of a therapeutically effective amount of a compound of formula (I) or a pharmaceutically acceptable salt, hydrate, solvate, prodrug, stereoisomer, or tautomer thereof, to a subject in need thereof. In some embodiments, the protein is IKZF1, IKZF2, or IKZF3.

In some embodiments, the present methods are directed to methods of treating diseases or disorders that would benefit from IKZF2 (Helios) degradation. In some embodiments, the disease or disorder is cancer. In some embodiments, the cancer is T cell leukemia, T cell lymphoma, Hodgkin's lymphoma, non-Hodgkin's lymphoma, myeloid leukemia, non-small cell lung cancer (NSCLC), melanoma, triple-negative breast cancer (TNBC), nasopharyngeal cancer (NPC), microsatellite stable colorectal cancer (mssCRC), thymoma, or carcinoid.

In some embodiments, the present methods are directed to methods of treating diseases or disorders that would benefit from thioredoxin-interacting protein (TXNIP) degradation. In some embodiments, the disease or disorder is gout, idiopathic pulmonary fibrosis, silicosis, asbestosis, nonalcoholic steatohepatitis, atherosclerosis, diabetes, diabetic nephropathy, diabetic retinopathy, or diabetic cardiomyopathy.

As demonstrated in the working examples, compounds of the present invention exhibit potent degradation of IKZF2 (Helios), or potent degradation of IKZF1 (Ikaros) and IKZF2.

Although not intending to be bound by any particular theory of operation, it is believed that inventive compounds may enhance an anti-tumor immune response by converting regulatory T cells into effector T cells, and by rescuing effector T cell function in exhausted T cells or CAR-T cells.

### BRIEF DESCRIPTION OF THE DRAWINGS

FIG. 1 is an immunoblot showing Helios degradation in Jurkat cells after 4 hours with treatment of two inventive compounds 36 and 65 at the indicated concentrations.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group, or a methyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

The terms “alkoxyl” or “alkoxy” as used herein refer to an alkyl group, as defined above, having an oxygen radical attached thereto, and which is the point of attachment. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group),“aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Unless stated otherwise, and to the extent not further defined for any particular group(s), any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1, 2, 3, or 4) heteroatoms such as oxygen, sulfur, and nitrogen grouped in a linear, branched, or cyclic structural format.

Representative examples of substituents may thus include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

In one aspect, compounds of the invention are represented by formula (I):

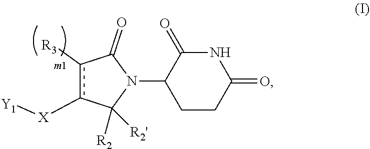

or a pharmaceutically acceptable salt, hydrate, solvate, prodrug, stereoisomer, or tautomer thereof,
wherein:
R₂ and R₂′ are independently hydrogen, (C₁-C₆)alkyl, or (C₁-C₆)haloalkyl, or
R₂ and R₂′ together form ═O;
each R₃ is independently hydrogen, halogen, (C₁-C₆)alkyl, or (C₁-C₆)haloalkyl;
X is —O—, —S—, —NR₆—, or —C(R₅)₂—;

### Y₁ is

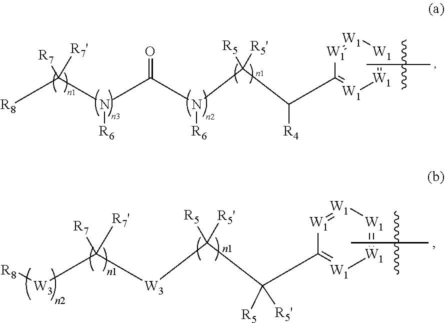

provided that when R₂ and R₂′ together form ═O, R₃ is H, X is NH, W₁ is CH, n₁ is 0, n₂ is 0, w₃ is NH, and R₈ is optionally substituted aryl, one of R₅ and R₅′ is not H,

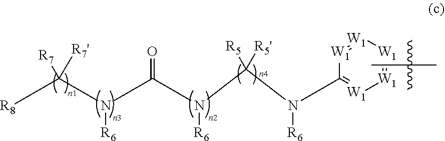

provided that when R₂ and R₂′ together form ═O, R₃ is H, X is NH, W₁ is CH, R₆ is H, n₄ is 1, n₂ is 0, n₃ is 1, R₆ is H, n₁ is 0, and R₈ is optionally substituted aryl, one of R₅ and R₅′ is not H,

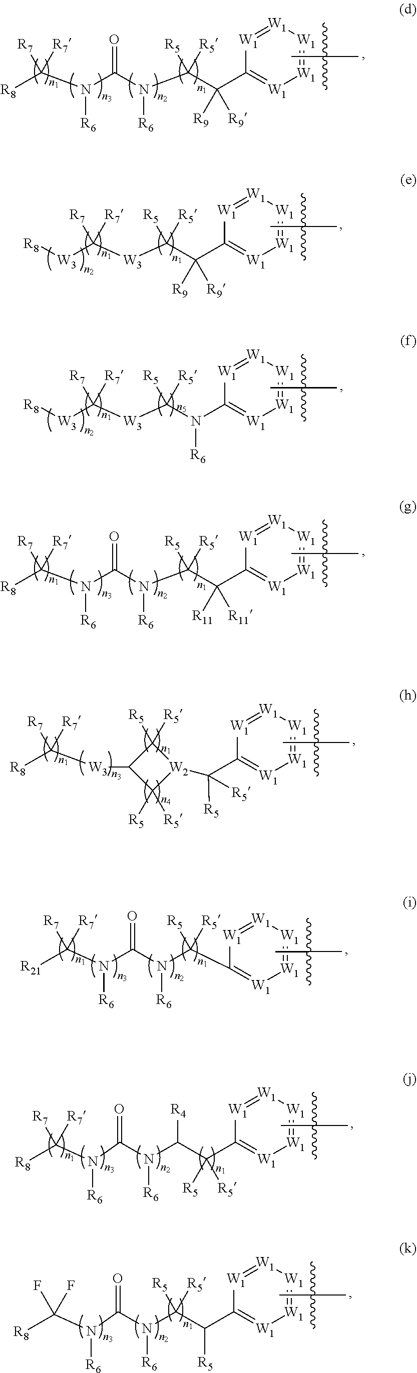

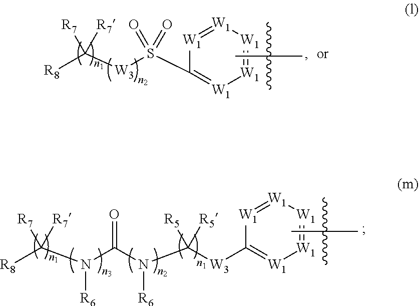

R₄ is —CF₃,

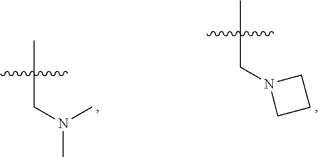

(C₃-C₇)alkyl, (C₃-C₇)haloalkyl, (C₃-C₇)cycloalkyl, 4- to 7-membered heterocycloalkyl, (C₆-C₁₀)aryl, monocyclic or bicyclic 5- to 10-membered heteroaryl, —N(R₆)₂, —OR₆, 4- to 7-membered heterocycloalkyl which contains at least one nitrogen atom and is linked via the nitrogen atom, (C₂-C₆)alkenyl, or (C₂-C₆)alkynyl; wherein said alkyl, cycloalkyl, heterocycloalkyl, aryl, or heteroaryl is further optionally and independently substituted by one or more identical or different R₁₀ groups;
R₅ and R₅′ are absent or independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups, or
R₅ and R₅′ together with the same carbon atom to which they are attached form a spiro (C₃-C₇)cycloalkyl group or a 4- to 7-membered heterocycloalkyl group, or
R₅ and R₅′, when on different carbon atoms, together with the atoms to which they are attached form a (C₃-C₇)cycloalkyl group or a 4- to 7-membered heterocycloalkyl group, or
R₅ and R₅′, when on adjacent atoms, together with the atoms to which they are attached form a (C₆-C₁₀)aryl or a 5- or 6-membered heteroaryl; wherein said cycloalkyl, heterocycloalkyl, aryl or heteroaryl is further optionally and independently substituted by one or more identical or different R₁₀ groups, or
R₅ and R₉, together with the atoms to which they are attached form a (C₃-C₇)cycloalkyl group or a 4- to 7-membered heterocycloalkyl group; wherein said cycloalkyl, or heterocycloalkyl is further optionally and independently substituted by one or more identical or different R₁₀ groups;
R₆ is hydrogen, (C₁-C₆)alkyl, (C₁-C₆)haloalkyl, (C₃-C₇)cycloalkyl, 4- to 7-membered heterocycloalkyl, (C₆-C₁₀)aryl, or monocyclic or bicyclic 5- to 10-membered heteroaryl; wherein said alkyl, cycloalkyl, heterocycloalkyl, aryl or heteroaryl is further optionally and independently substituted by one or more identical or different R₁₀ groups, or
two R₆ when on different nitrogen atoms, together with the atoms to which they are attached form a 4- to 7-membered heterocycloalkyl group; wherein said heterocycloalkyl is further optionally and independently substituted by one or more identical or different R₁₀ groups, or
R₆ and R₇, together with the atoms to which they are attached form a 4- to 8-membered heterocycloalkyl group; wherein said heterocycloalkyl is further optionally and independently substituted by one or more identical or different R₁₀ groups;
R₇ and R₇′ are absent or independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups, or
R₇ and R₇′ together with the same carbon atom to which they are attached form a spiro (C₃-C₇)cycloalkyl group or a 4- to 7-membered heterocycloalkyl group, or
R₇ and R₇′, when on different carbon atoms, together with the atoms to which they are attached form a (C₃-C₇)cycloalkyl group or a 4- to 7-membered heterocycloalkyl group, or
R₇ and R₇′, when on adjacent atoms, together with the atoms to which they are attached form a (C₆-C₁₀)aryl or a 5- or 6-membered heteroaryl; wherein said cycloalkyl, heterocycloalkyl, aryl or heteroaryl is further optionally and independently substituted by one or more identical or different R₁₀ groups;
R₈ is a (C₆-C₁₀)aryl, or monocyclic or bicyclic 5- to 10-membered heteroaryl; wherein said aryl or heteroaryl is further optionally and independently substituted by one or more identical or different groups selected from R₁₀ and

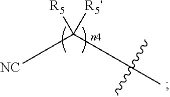

R₉ and R₉′ are independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups, or
R₉ and R₅, together with the atoms to which they are attached form a (C₃-C₇)cycloalkyl group or a 4- to 7-membered heterocycloalkyl group; wherein said cycloalkyl, or heterocycloalkyl is further optionally and independently substituted by one or more identical or different R₁₀ groups, or
R₉ and R₉′ together with the same carbon atom to which they are attached form a spiro (C₃-C₇)cycloalkyl group or a 4- to 7-membered heterocycloalkyl group;
each R₁₀ is independently alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, or heteroaryl, or
two adjacent R₁₀ groups taken together with the respective atoms to which each is attached form aryl, heteroaryl, 5- to 8-membered cycloalkyl, or 5- to 8-membered heterocycloalkyl;
R₁₁ and R₁₁′ are independently (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups, provided that R₁₁ and R₁₁′ are both not methyl;
R₂₁ is a monocyclic or bicyclic 5- to 10-membered heteroaryl; wherein said heteroaryl is further optionally and independently substituted by one or more identical or different R₁₀ groups;
m₁ is 0, 1, or 2;
n₁ is independently 0, 1, 2, or 3;
n₂ and n₃ are independently 0 or 1, provided that n₂ and n₃ cannot both be 0;
n₄ is 1, 2, 3, 4, or 5;
n₅ is 2, 3, 4, or 5;
W₁ is N, CR₅, or a carbon atom that is the point of attachment;
W₂ is N or CR₅; and
W₃ is —O—, —S—, —NR₆—, or —S(O₂)—.

In some embodiments, formula I is of formula I-1:

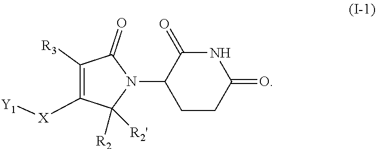

In some embodiments, formula I is of formula I-2:

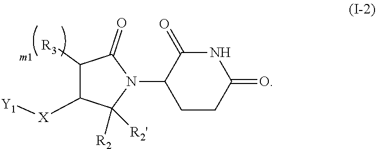

In some embodiments, R₂ and R₂′ are each hydrogen.

In some embodiments, R₂ and R₂′ together form ═O.

In some embodiments, R₃ is hydrogen.

In some embodiments, R₃ is halogen.

In some embodiments, X is —O—.

In some embodiments, X is —S—.

In some embodiments, X is —NR₆—.

In some embodiments, X is —C(R₅)₂—.

In some embodiments, Y₁ is

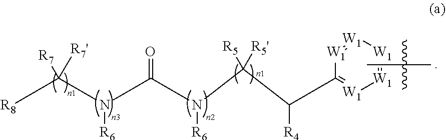

In some embodiments, Y₁ is

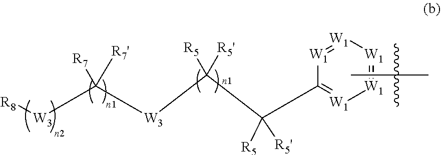

provided that when R₂ and R₂′ together form ═O, R₃ is H, X is NH, W₁ is CH, n₁ is 0, n₂ is 0, W₃ is NH, and R₈ is optionally substituted aryl, one of R₅ and R₅′ is not H.

In some embodiments, Y₁ is

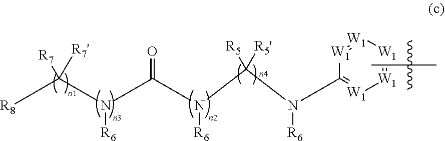

provided that when R₂ and R₂′ together form ═O, R₃ is H, X is NH, W₁ is CH, R₆ is H, n₄ is 1, n₂ is 0, n₃ is 1, R₆ is H, n₁ is 0, and R₈ is optionally substituted aryl, one of R₅ and R₅′ is not H.

In some embodiments, Y₁ is

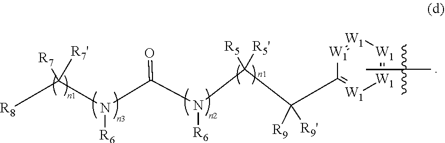

In some embodiments, Y₁ is

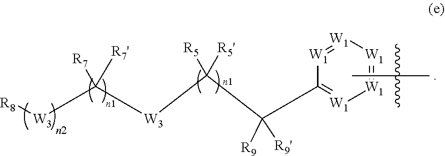

In some embodiments, Y₁ is

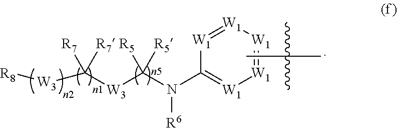

In some embodiments, Y₁ is

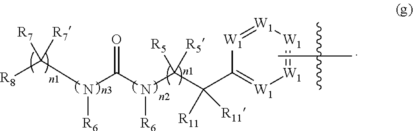

In some embodiments, Y₁ is

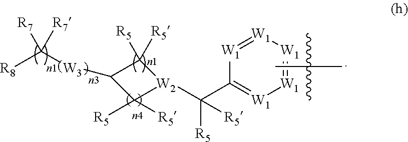

In some embodiments, Y₁ is

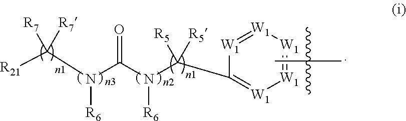

In some embodiments, Y₁ is

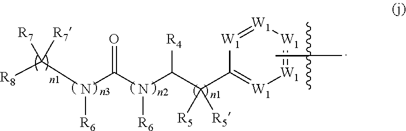

In some embodiments, Y₁ is

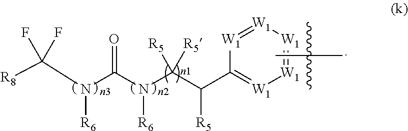

In some embodiments, Y₁ is

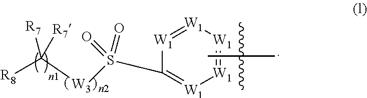

In some embodiments, Y₁ is

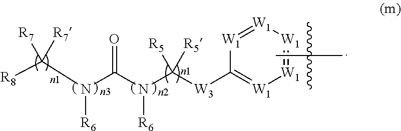

In some embodiments, R₄ is (C₃-C₇)alkyl, (C₃-C₇)haloalkyl, (C₃-C₇)cycloalkyl, 4- to 7-membered heterocycloalkyl, (C₆-C₁₀)aryl, monocyclic or bicyclic 5- to 10-membered heteroaryl, —N(R₆)₂, —OR₆, 4- to 7-membered heterocycloalkyl which contains at least one nitrogen atom and is linked via the nitrogen atom, (C₂-C₆)alkenyl, or (C₂-C₆)alkynyl; wherein said alkyl, cycloalkyl, heterocycloalkyl, aryl, or heteroaryl is further optionally and independently substituted by one or more identical or different R₁₀ groups.

In some embodiments, R₅ and R₅′ are absent or independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups.

In some embodiments, R₅ and R₅′ are independently hydrogen, methyl, ethyl, isopropyl, phenyl, chloro, fluoro, —NH₂, —NMe₂, —CF₃

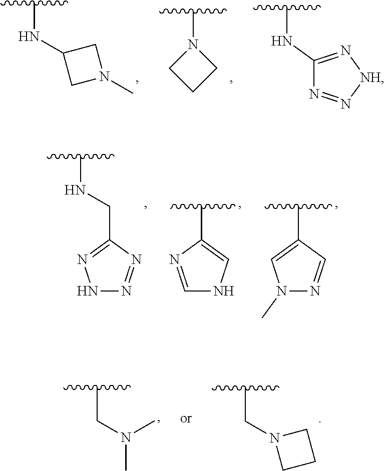

In some embodiments, R₅ and R₅′ together with the same carbon atom to which they are attached form a spiro (C₃-C₇)cycloalkyl group or a 4- to 7-membered heterocycloalkyl group.

In some embodiments, R₅ and R₅′ together with the same carbon atom to which they are attached form a spiro-cycle which is a cyclopropyl, cyclobutyl, oxetane, azetidine, or tetrahydropyran spiro-cycle.

In some embodiments, R₅ and R₅′, when on different carbon atoms, together with the atoms to which they are attached form a (C₃-C₇)cycloalkyl group or a 4- to 7-membered heterocycloalkyl group; wherein said cycloalkyl or heterocycloalkyl is further optionally and independently substituted by one or more identical or different R₁₀ groups.

In some embodiments, R₅ and R₅′ when on different carbon atoms, together with the atoms to which they are attached form a cyclopropyl or cyclobutyl ring.

In some embodiments, R₅ and R₅′, when on adjacent atoms, together with the atoms to which they are attached form a (C₆-C₁₀)aryl or a 5- or 6-membered heteroaryl; wherein said aryl or heteroaryl is further optionally and independently substituted by one or more identical or different R₁₀ groups.

In some embodiments, R₆ is hydrogen, (C₁-C₆)alkyl, (C₁-C₆)haloalkyl, (C₃-C₇)cycloalkyl, 4- to 7-membered heterocycloalkyl, (C₆-C₁₀)aryl, or monocyclic or bicyclic 5- to 10-membered heteroaryl; wherein said alkyl, cycloalkyl, heterocycloalkyl, aryl or heteroaryl is further optionally and independently substituted by one or more identical or different R₁₀ groups.

In some embodiments, two R₆ when on different nitrogen atoms, together with the atoms to which they are attached form a 4- to 7-membered heterocycloalkyl group; wherein said heterocycloalkyl is further optionally and independently substituted by one or more identical or different R₁₀ groups.

In some embodiments, R₆ and R₇, together with the atoms to which they are attached form a 4- to 8-membered heterocycloalkyl group; wherein said heterocycloalkyl is further optionally and independently substituted by one or more identical or different R₁₀ groups.

In some embodiments, R₇ and R₇′ are absent or independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups.

In some embodiments, R₇ and R₇′ are independently hydrogen, methyl, ethyl, isopropyl, phenyl, chloro, fluoro, —NH₂, —NMe₂, —CF₃

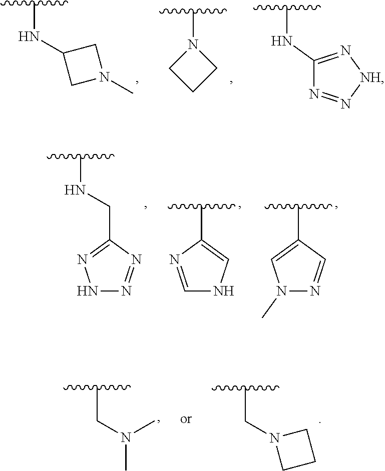

In some embodiments, R₇ and R₇′ together with the same carbon atom to which they are attached form a spiro (C₃-C₇)cycloalkyl group or a 4- to 7-membered heterocycloalkyl group; wherein said cycloalkyl or heterocycloalkyl is further optionally and independently substituted by one or more identical or different R₁₀ groups.

In some embodiments, R₇ and R₇′ together with the same carbon atom to which they are attached form a spiro-cycle which is a cyclopropyl, cyclobutyl, oxetane, azetidine, or tetrahydropyran spiro-cycle.

In some embodiments, R₇ and R₇′, when on different carbon atoms, together with the atoms to which they are attached form a (C₃-C₇)cycloalkyl group or a 4- to 7-membered heterocycloalkyl group; wherein said cycloalkyl or heterocycloalkyl is further optionally and independently substituted by one or more identical or different R₁₀ groups.

In some embodiments, R₇ and R₇′ when on different carbon atoms, together with the atoms to which they are attached form a cyclopropyl or cyclobutyl ring.

In some embodiments, R₇ and R₇′, when on adjacent atoms, together with the atoms to which they are attached form a (C₆-C₁₀)aryl or a 5- or 6-membered heteroaryl; wherein said aryl or heteroaryl is further optionally and independently substituted by one or more identical or different R₁₀ groups.

In some embodiments, R₈ is a (C₆-C₁₀)aryl; wherein said aryl is further optionally and independently substituted by one or more identical or different groups selected from R₁₀ and

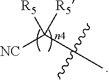

In some embodiments, R₈ is monocyclic or bicyclic 5- to 10-membered heteroaryl; wherein said heteroaryl is further optionally and independently substituted by one or more identical or different groups selected from R₁₀ and

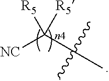

In some embodiments, R₈ is

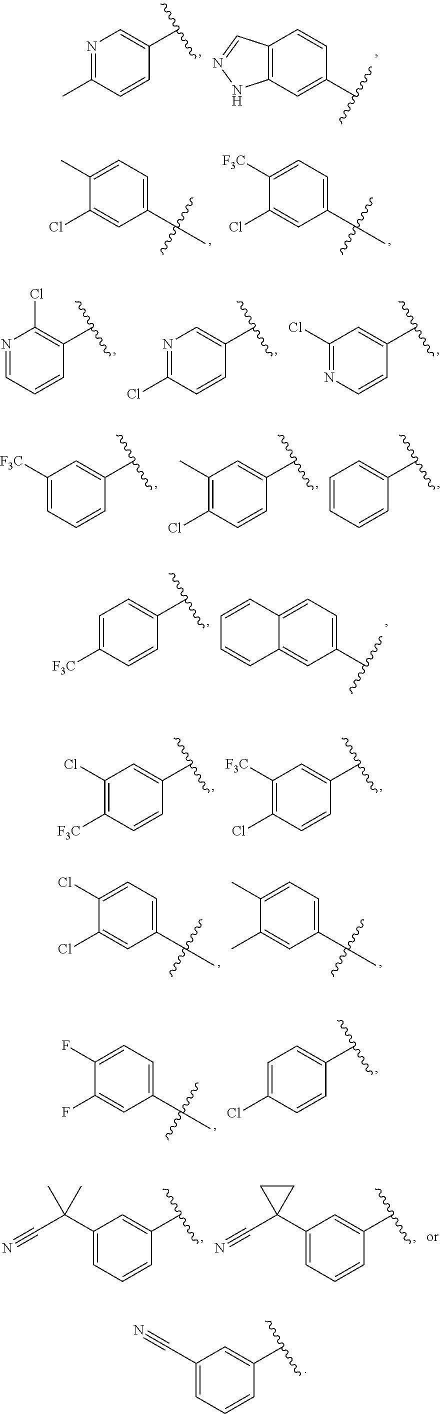

In some embodiments, R₉ and R₉′ are independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups.

In some embodiments, R₉ and R₉′ are independently hydrogen, methyl, ethyl, isopropyl, phenyl, chloro, fluoro, —NH₂, —NMe₂, —CF₃

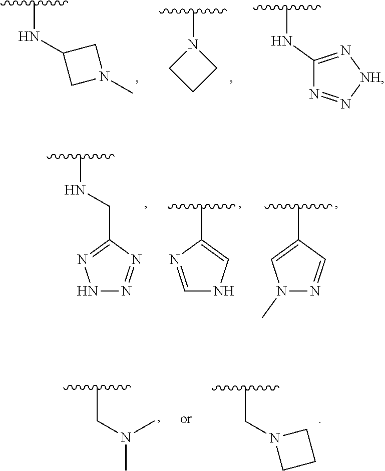

In some embodiments, R₉ and R₅, together with the atoms to which they are attached form a (C₃-C₇)cycloalkyl group or a 4- to 7-membered heterocycloalkyl group; wherein said cycloalkyl, or heterocycloalkyl is further optionally and independently substituted by one or more identical or different R₁₀ groups.

In some embodiments, R₉ and R₉′ together with the same carbon atom to which they are attached form a spiro (C₃-C₇)cycloalkyl group or a 4- to 7-membered heterocycloalkyl group.

In some embodiments, R₉ and R₉′ together with the same carbon atom to which they are attached form a spiro-cycle which is a cyclopropyl, cyclobutyl, oxetane, azetidine, or tetrahydropyran spiro-cycle.

In some embodiments, each R₁₀ is independently alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, or heteroaryl.

In some embodiments, two adjacent R₁₀ groups taken together with the respective atoms to which each is attached form aryl, heteroaryl, 5- to 8-membered cycloalkyl, or 5- to 8-membered heterocycloalkyl.

In some embodiments, R₁₁ and R₁₁′ are independently (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, or (C₂-C₆)alkenyl; wherein said alkyl, alkynyl, cycloalkyl, heterocycloalkyl, aryl, or heteroaryl is further optionally and independently substituted by one or more identical or different R₁₀ groups, provided that R₁₁ and R₁₁′ are both not methyl.

In some embodiments, R₂₁ is a monocyclic 5- to 10-membered heteroaryl; wherein said heteroaryl is further optionally and independently substituted by one or more identical or different R₁₀ groups.

In some embodiments, R₂₁ is a bicyclic 5- to 10-membered heteroaryl; wherein said heteroaryl is further optionally and independently substituted by one or more identical or different R₁₀ groups.

In some embodiments, R₂₁ is

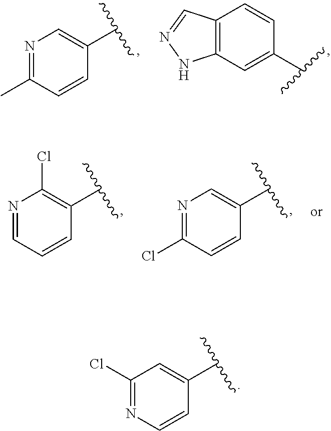

In some embodiments, m₁ is 0.

In some embodiments, m₁ is 1.

In some embodiments, m₁ is 2.

In some embodiments, n₁ is 0.

In some embodiments, n₁ is 1.

In some embodiments, n₁ is 2.

In some embodiments, n₁ is 3.

In some embodiments, n₂ is 0.

In some embodiments, n₂ is 1.

In some embodiments, n₃ is 0.

In some embodiments, n₃ is 1.

In some embodiments, n₄ is 1.

In some embodiments, n₄ is 2.

In some embodiments, n₄ is 3.

In some embodiments, n₄ is 4.

In some embodiments, n₄ is 5.

In some embodiments, n₅ is 2.

In some embodiments, n₅ is 3.

In some embodiments, n₅ is 4.

In some embodiments, n₅ is 5.

In some embodiments, W₁ is N.

In some embodiments, W₁ is CR₅.

In some embodiments, W₁ is a carbon atom that is the point of attachment.

In some embodiments, W₂ is N.

In some embodiments, W₂ is CR₅.

In some embodiments, W₃ is —O—.

In some embodiments, W₃ is —S—.

In some embodiments, W₃ is —NR₆—.

In some embodiments, W₃ is —S(O)₂—.

In some embodiments, the compound of formula (I) is represented by any of formulas Ia-m:

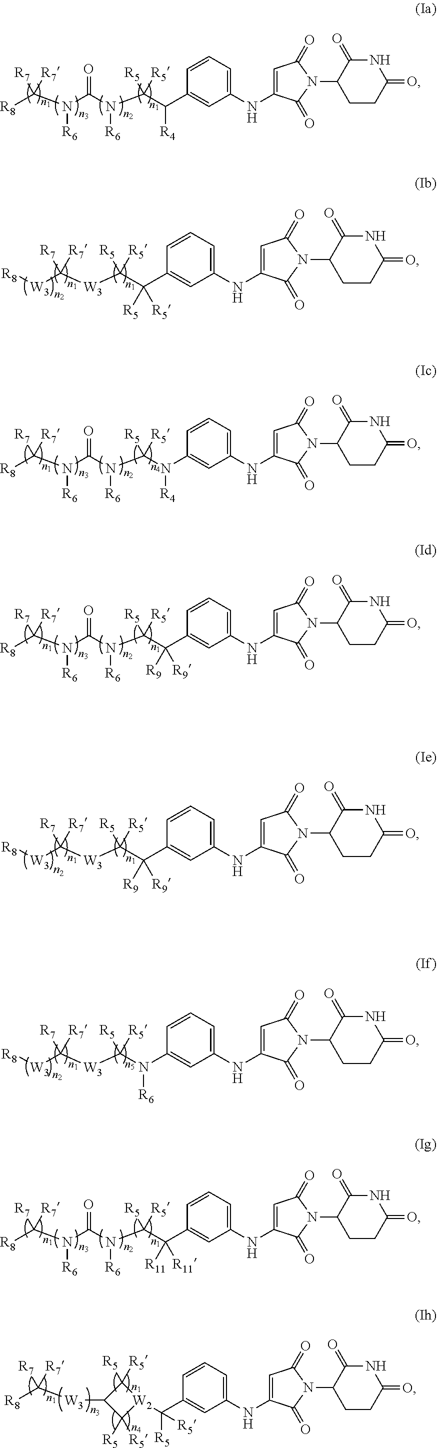

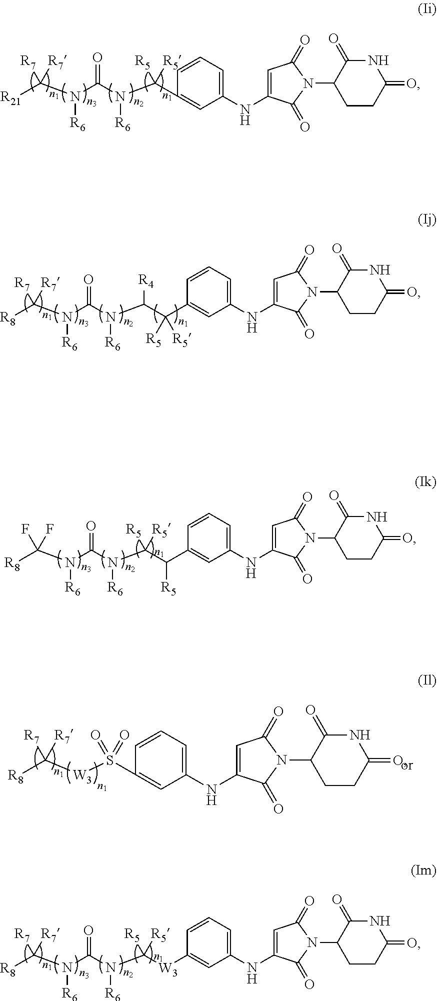

or a pharmaceutically acceptable salt, hydrate, solvate, prodrug, stereoisomer, or tautomer thereof.

In some embodiments, the compound of formula (Ia) is:

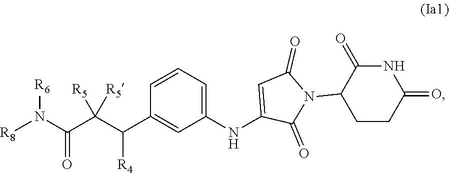

or a pharmaceutically acceptable salt, hydrate, solvate, prodrug, stereoisomer, or tautomer thereof,
wherein
R₆ is hydrogen or (C₁-C₆)alkyl; and
R₄, R₅, R₅′, and R₈ as defined as above.

In some embodiments, the compound of formula (Ib) is:

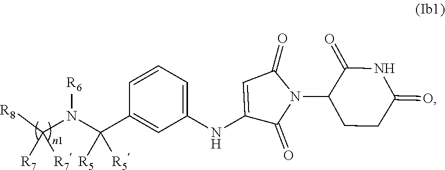

or a pharmaceutically acceptable salt, hydrate, solvate, prodrug, stereoisomer, or tautomer thereof,
wherein
R₆ is hydrogen or (C₁-C₆)alkyl;
n₁ is 1 or 2; and
R₅, R₅′, R₇, R₇′, and R₈ as defined as above.

In some embodiments, the compound of formula (Id) is:

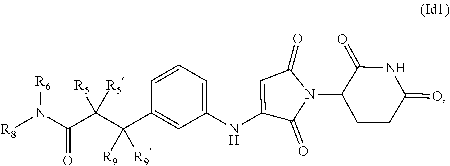

or a pharmaceutically acceptable salt, hydrate, solvate, prodrug, stereoisomer, or tautomer thereof,
wherein
R₆ is hydrogen or (C₁-C₆)alkyl;
R₉ and R₅, together with the atoms to which they are attached form a (C₃-C₇)cycloalkyl group or a 4- to 7-membered heterocycloalkyl group; wherein said cycloalkyl, or heterocycloalkyl is further optionally and independently substituted by one or more identical or different R₁₀ groups; and
R₅′, R₉′, and R₈ as defined as above.

In some embodiments, the compound of formula (Ii) is:

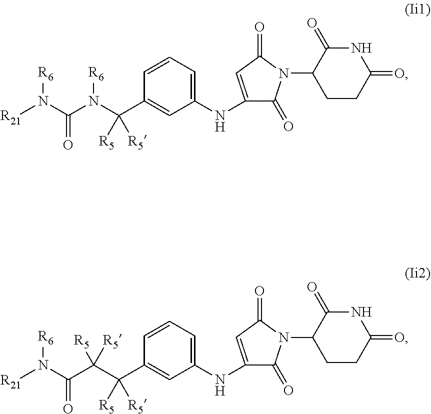

or a pharmaceutically acceptable salt, hydrate, solvate, prodrug, stereoisomer, or tautomer thereof,
wherein
R₆ is hydrogen or (C₁-C₆)alkyl;
R₂₁ is a monocyclic 6-membered heteroaryl; wherein said heteroaryl is further optionally and independently substituted by one or more identical or different R₁₀ groups; and
R₅ and R₅′ as defined as above.

In some embodiments, the compound of formula (Ij) is:

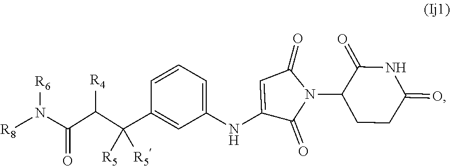

or a pharmaceutically acceptable salt, hydrate, solvate, prodrug, stereoisomer, or tautomer thereof,
wherein
R₆ is hydrogen or (C₁-C₆)alkyl; and
R₄, R₅, R₅′, and R₈ as defined as above.

Representative examples of compounds of the invention have the following structures:

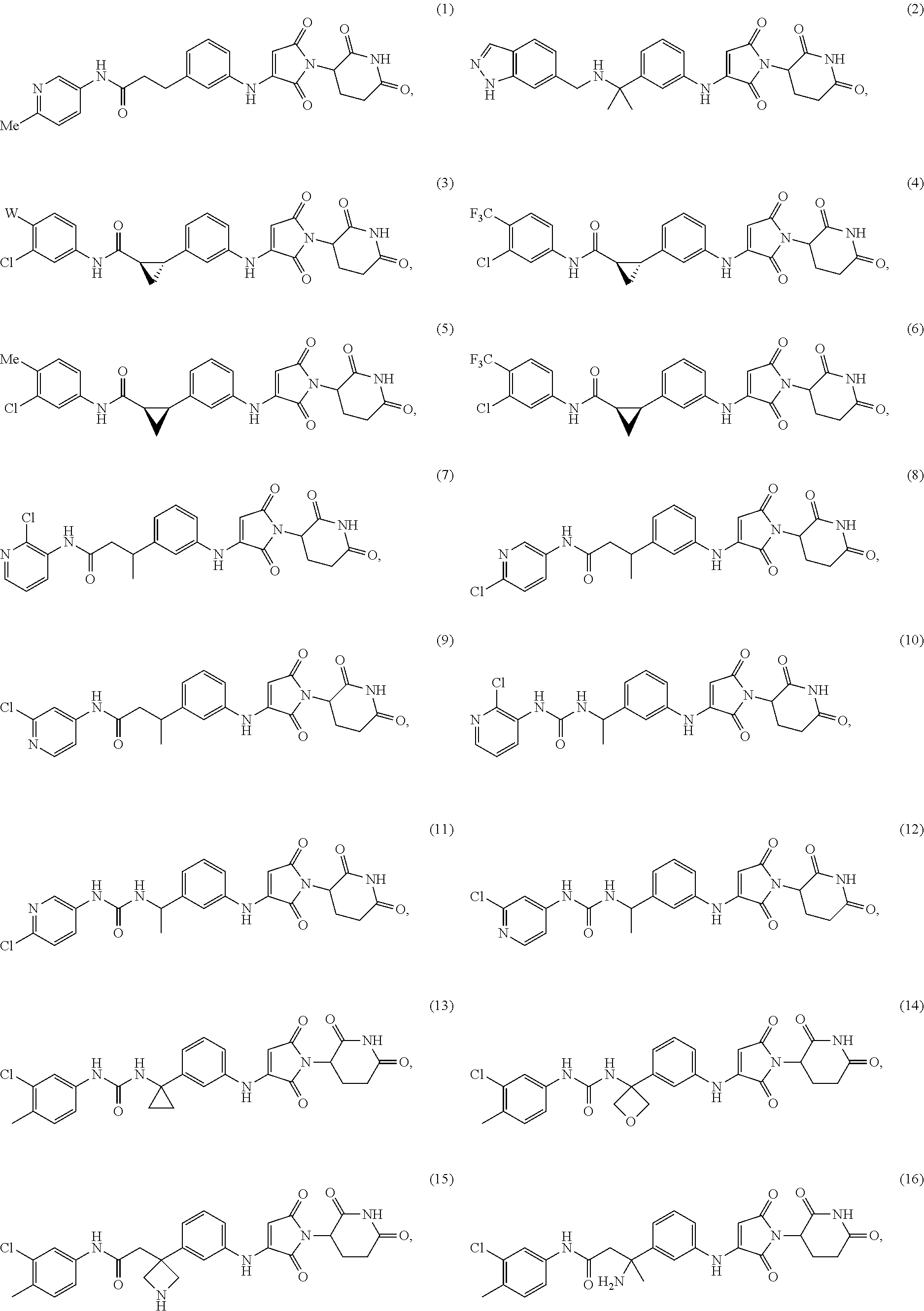

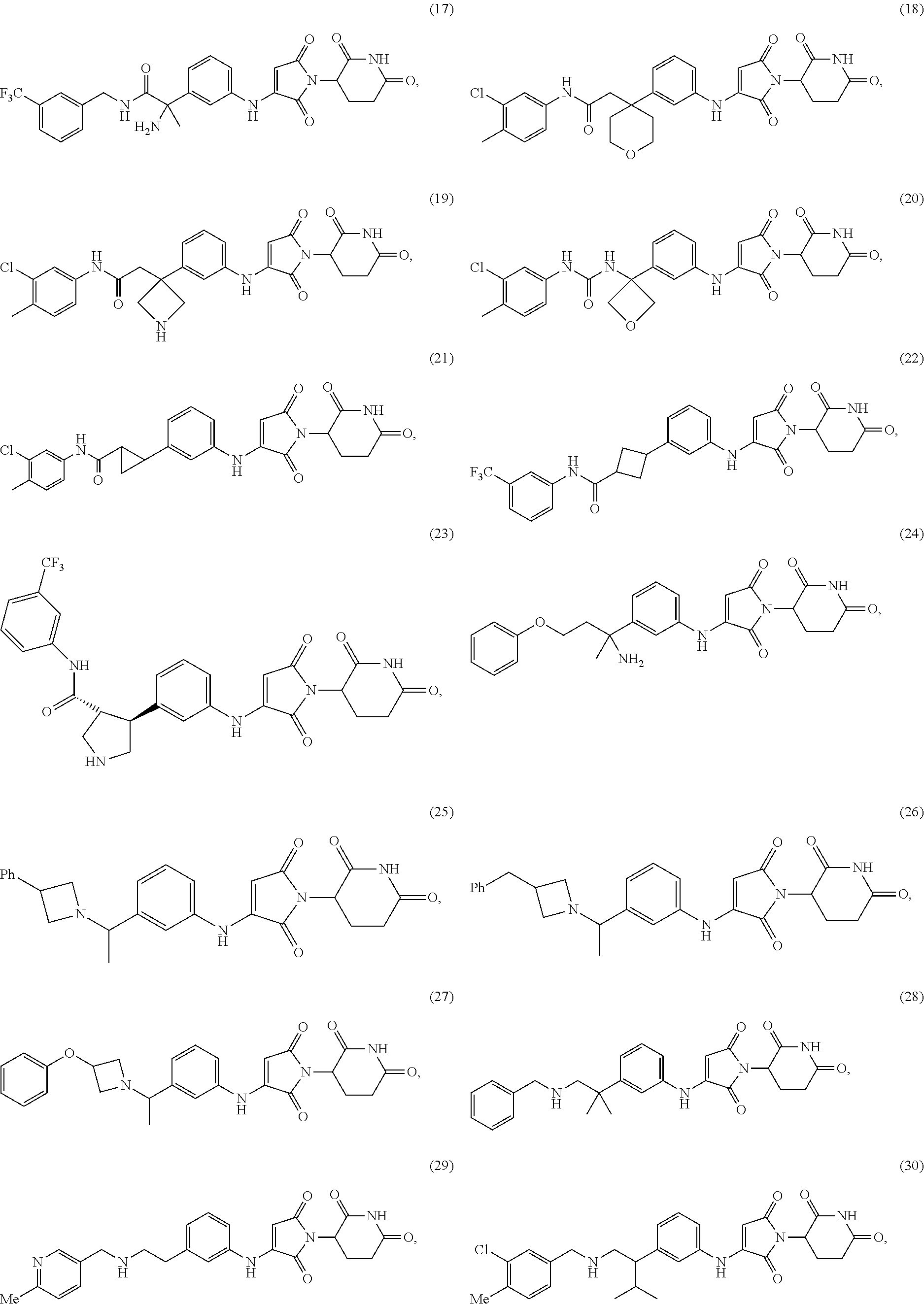

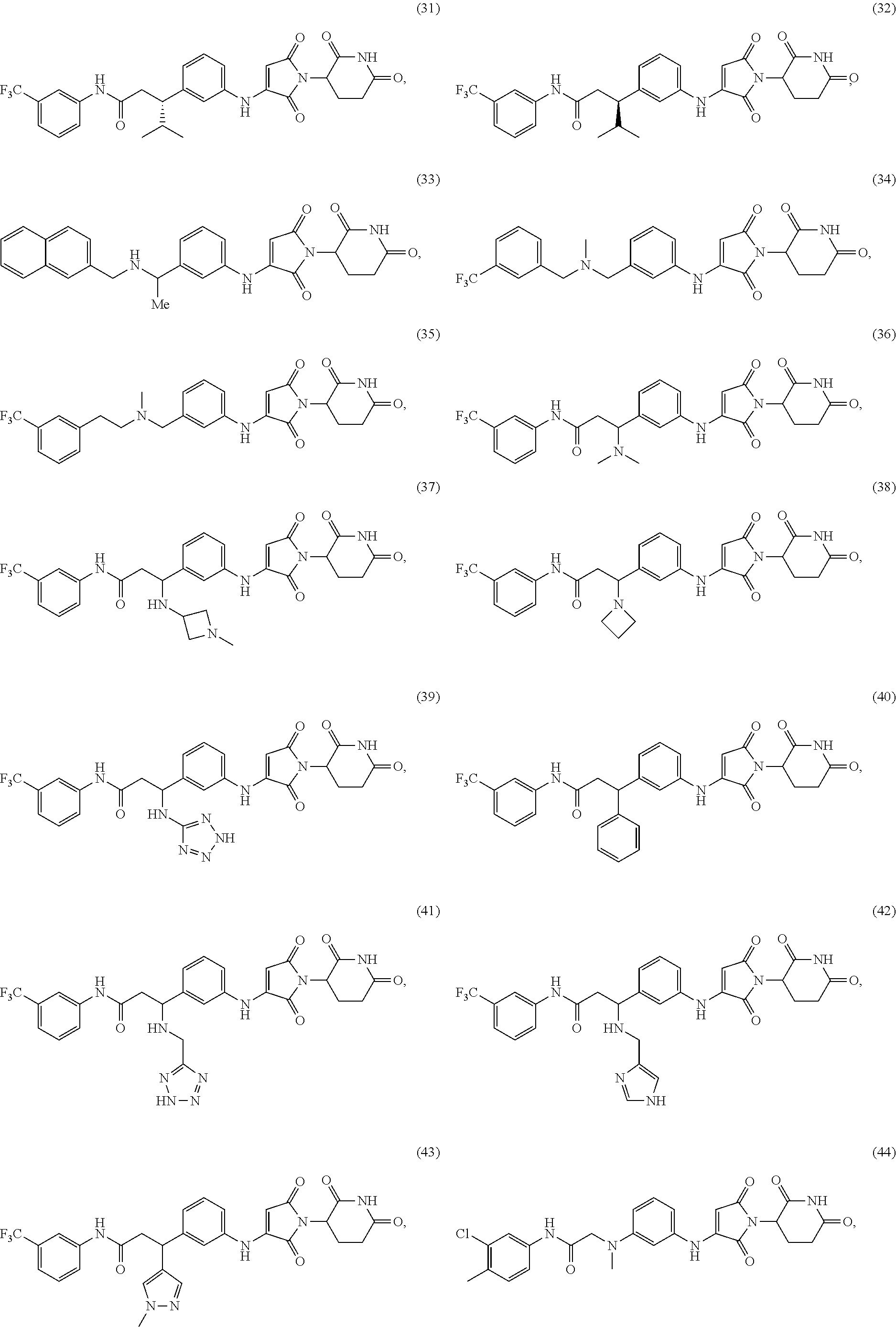

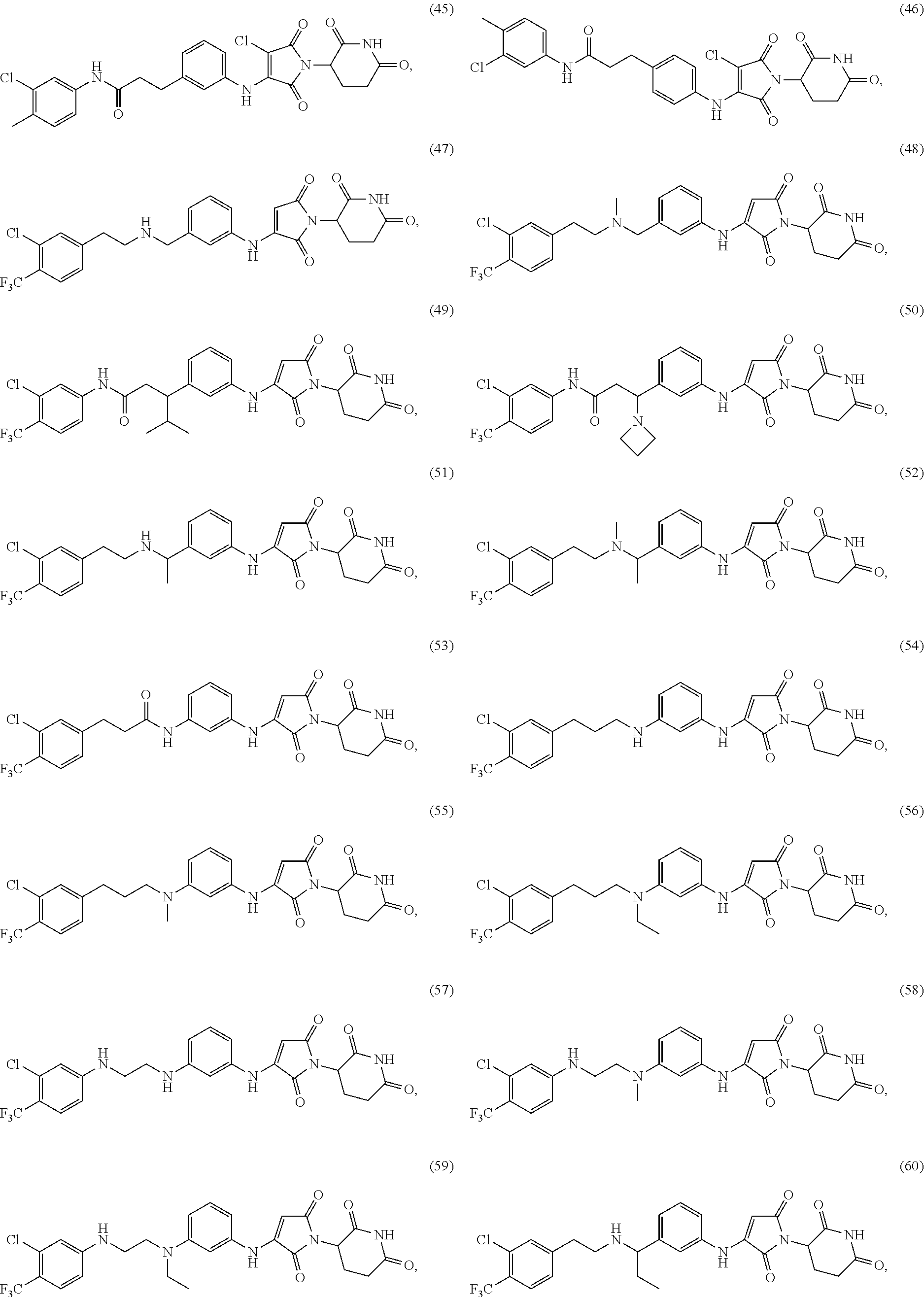

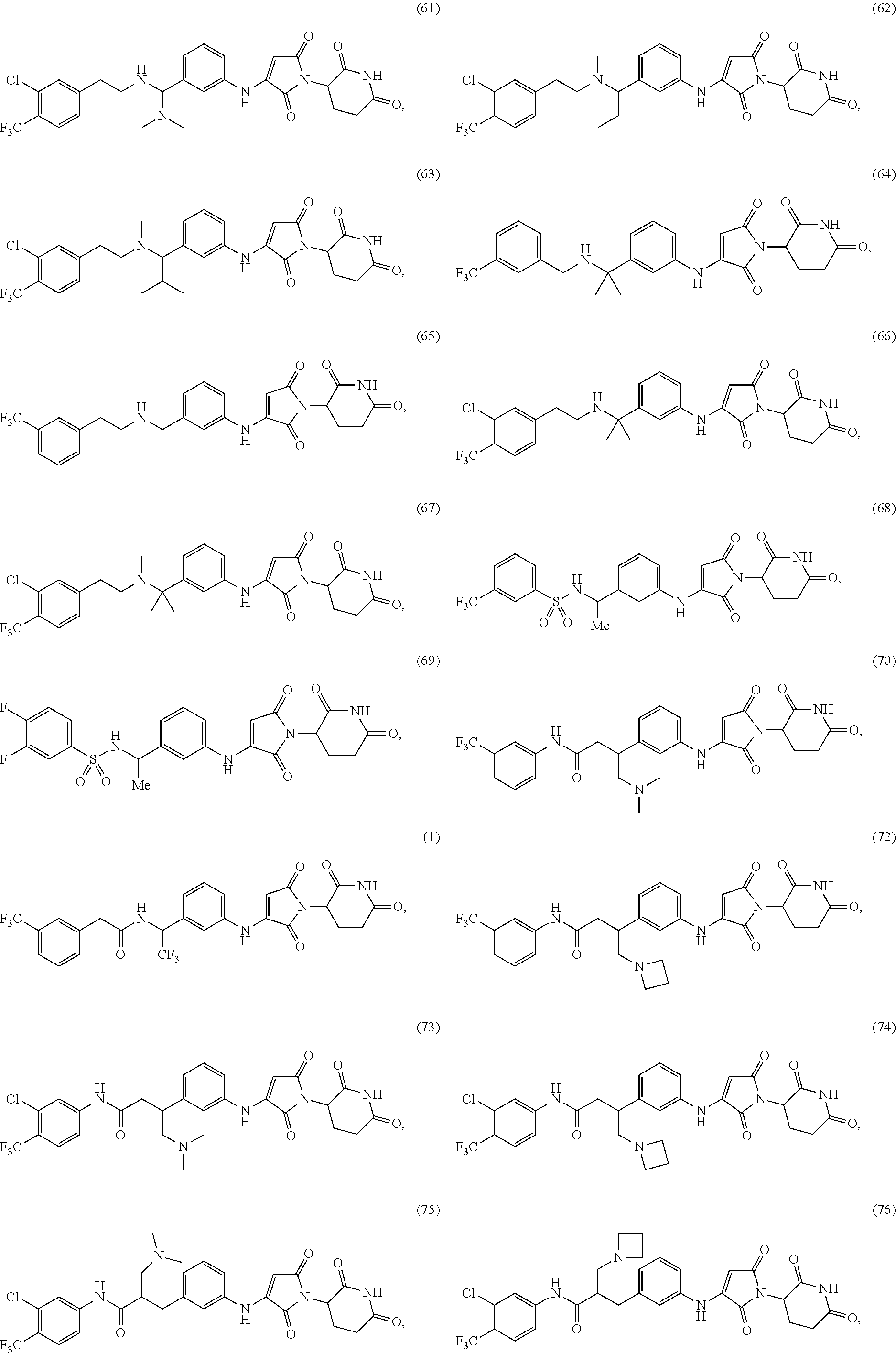

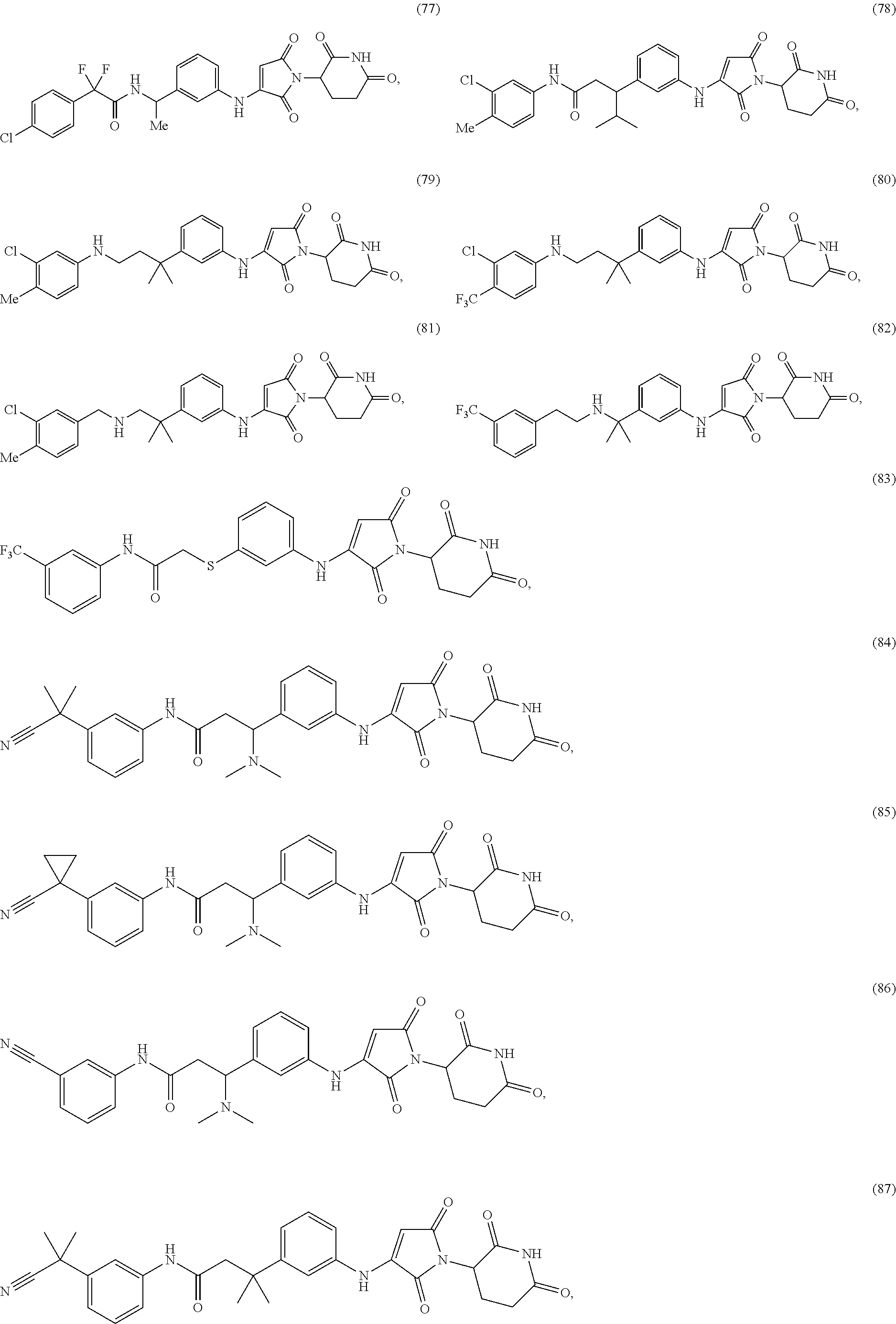

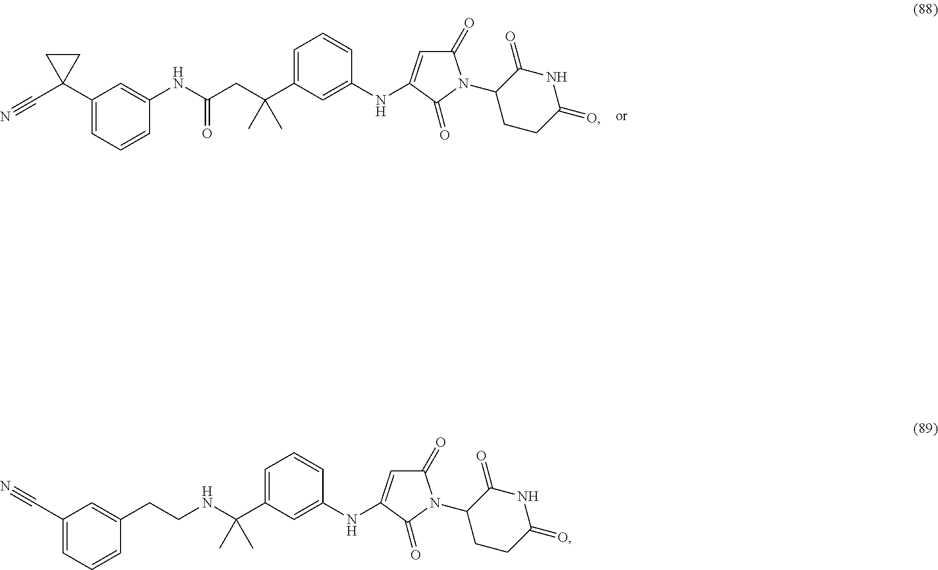 [SMILES: CC(C)(CC(=O)Nc1cccc(C2(C#N)CC2)c1)c1cccc(NC2=CC(=O)N(C3CCC(=O)NC3=O)C2=O)c1 ; CC(C)(NCCc1cccc(C#N)c1)c1cccc(NC2=CC(=O)N(C3CCC(=O)NC3=O)C2=O)c1]

or a pharmaceutically acceptable salt, hydrate, solvate, prodrug, stereoisomer, or tautomer thereof.

Compounds of the present invention may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or abase. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin. Suitable base salts include aluminum, calcium, lithium, magnesium, potassium, sodium, or zinc salts.

Compounds of the present invention may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In some embodiments, the compound is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

Thus, the term compounds of formula (I) embraces the free base form of the compounds, as well as isotopic derivatives, N-oxides, crystalline forms (also known as polymorphs), active metabolites of the compounds having the same type of activity, prodrugs, tautomers, and unsolvated as well as solvated (e.g., hydrated) forms with pharmaceutically acceptable solvents such as water, ethanol, and the like, of the compounds.

The compounds of the present invention may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

Thus, in addition to salts and stereoisomers, the “compounds” of the present invention encompass their isotopic derivatives, their tautomeric forms, their polymorphs, their pharmaceutically acceptable hydrates, their pharmaceutically acceptable prodrugs, and their pharmaceutically acceptable solvates.

In some embodiments, the pharmaceutical composition comprises a co-crystal of an inventive compound. The term “co-crystal”, as used herein, refers to a stoichiometric multi-component system comprising a compound of the invention and a co-crystal former wherein the compound of the invention and the co-crystal former are connected by non-covalent interactions. The term “co-crystal former”, as used herein, refers to compounds which can form intermolecular interactions with a compound of the invention and co-crystallize with it. Representative examples of co-crystal formers include benzoic acid, succinic acid, fumaric acid, glutaric acid, trans-cinnamic acid, 2,5-dihydroxybenzoic acid, glycolic acid, trans-2-hexanoic acid, 2-hydroxycaproic acid, lactic acid, sorbic acid, tartaric acid, ferulic acid, suberic acid, picolinic acid, salicyclic acid, maleic acid, saccharin, 4,4′-bipyridine p-aminosalicyclic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present invention is directed to a method for making an inventive compound, or a pharmaceutically acceptable salt, hydrate, solvate, prodrug, stereoisomer, or tautomer thereof. Broadly, the inventive compounds or a pharmaceutically acceptable salt, hydrate, solvate, prodrug, stereoisomer, or tautomer thereof may be prepared by any process known to be applicable to the preparation of chemically related compounds. The compounds of the present invention will be better understood in connection with the synthetic schemes that described in various working examples and which illustrate non-limiting methods by which the compounds of the invention may be prepared.

### Pharmaceutical Compositions

Another aspect of the present invention is directed to a pharmaceutical composition that includes a therapeutically effective amount of a compound of formula (I) or a pharmaceutically acceptable salt, hydrate, solvate, prodrug, stereoisomer, or tautomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of formula (I) and their pharmaceutically acceptable salts, hydrates, solvates, prodrugs, stereoisomers, or tautomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrastemal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds are formulated for oral or intravenous administration (e.g., systemic intravenous injection).

Accordingly, compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (I) may be formulated for topical administration which as used herein, refers to administration intradermally by invention of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of an inventive compound or a pharmaceutically acceptable salt, hydrate, solvate, prodrug, stereoisomer, or tautomer thereof that is effective in producing the desired therapeutic response in a patient suffering from a disease or disorder characterized or mediated by activity of a protein that is a substrate for a complex between cereblon (CRBN) and an inventive compound. The term “therapeutically effective amount” thus includes the amount of the inventive compound or a pharmaceutically acceptable salt, hydrate, solvate, prodrug, stereoisomer, or tautomer thereof, that when administered, induces a positive modification in the disease or disorder to be treated, is sufficient to prevent development or progression of the disease or disorder, or alleviate to some extent, one or more of the symptoms of the disease or disorder being treated in a subject, or which simply kills or inhibits the growth of diseased cells, or reduces the protein that is a substrate for a complex between cereblon (CRBN) in diseased cells. In some embodiments, the disease or disorder is characterized or mediated by activity of IKZF1, IKZF2 (Helios), or IKZF3. In some embodiments, the disease or disorder is mediated by IKZF2 activity.

The total daily dosage of the compounds and usage thereof may be decided in accordance with standard medical practice, e.g., by the attending physician using sound medical judgment. The specific therapeutically effective dose for any particular subject will depend upon a variety of factors, including the following: the disease or disorder being treated and the severity thereof (e.g., its present status); the activity of the compound employed; the specific composition employed; the age, body weight, general health, sex and diet of the subject; the time of administration, route of administration, and rate of excretion of the compound employed; the duration of the treatment; drugs used in combination or coincidental with the specific compound employed; and like factors well known in the medical arts (see, for example, Hardman et al., eds., Goodman and Gilman's The Pharmacological Basis of Therapeutics, 10th Edition, McGraw-Hill Press, 155-173, 2001).

Compounds of formula (I) may be effective over a wide dosage range. In some embodiments, the total daily dosage (e.g., for adult humans) may range from about 0.001 to about 1600 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the compound may be administered at a dose in range from about 0.01 mg to about 200 mg/kg of body weight per day. In some embodiments, a dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

### Methods of Use

In another aspect, the compounds and pharmaceutically acceptable salts and stereoisomers of the present invention may be useful in the treatment of diseases and disorders characterized or mediated by activity of a protein that is a substrate for a complex between CRBN and an inventive compound, and which protein participates in the inception or manifestation of one or more symptoms or markers, severity or progression of the disease or disorder, and/or wherein the degradation of the targeted protein may confer a therapeutic benefit.

A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

In some embodiments, the disease or disorder is characterized or mediated by activity of IKZF1, IKZF2, or IKZF3. In some embodiments, the disease or disorder is mediated by IKZF2 activity and the methods entail administration of a therapeutically effective amount of a compound of formula (I), or a pharmaceutically acceptable salt, hydrate, solvate, prodrug, stereoisomer, or tautomer thereof, to a subject in need thereof.

In some embodiments, the present methods treat diseases or disorders involving TXNIP, and entail administration of a therapeutically effective amount of a compound of formula (I), or a pharmaceutically acceptable salt, hydrate, solvate, prodrug, stereoisomer, or tautomer thereof, to a subject in need thereof.

In some embodiments, compounds of formula (I) may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present invention may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

Exemplary types of non-cancerous (e.g., cell proliferative) diseases or disorders that may be amenable to treatment with the compounds of the present invention include inflammatory diseases and conditions, autoimmune diseases, neurodegenerative diseases, heart diseases, viral diseases, chronic and acute kidney diseases or injuries, metabolic diseases, and allergic and genetic diseases.

Representative examples of specific non-cancerous diseases and disorders include rheumatoid arthritis, alopecia areata, lymphoproliferative conditions, autoimmune hematological disorders (e.g. hemolytic anemia, aplastic anemia, anhidrotic ectodermal dysplasia, pure red cell anemia and idiopathic thrombocytopenia), cholecystitis, acromegaly, rheumatoid spondylitis, osteoarthritis, gout, scleroderma, sepsis, septic shock, dacryoadenitis, cryopyrin associated periodic syndrome (CAPS), endotoxic shock, endometritis, gram-negative sepsis, keratoconjunctivitis sicca, toxic shock syndrome, asthma, adult respiratory distress syndrome, chronic obstructive pulmonary disease, chronic pulmonary inflammation, chronic graft rejection, hidradenitis suppurativa, inflammatory bowel disease, Crohn's disease, Behcet's syndrome, systemic lupus erythematosus, glomerulonephritis, multiple sclerosis, juvenile-onset diabetes, autoimmune uveoretinitis, autoimmune vasculitis, thyroiditis, Addison's disease, lichen planus, appendicitis, bullous pemphigus, pemphigus vulgaris, pemphigus foliaceus, paraneoplastic pemphigus, myasthenia gravis, immunoglobulin A nephropathy, Hashimoto's disease, Sjogren's syndrome, vitiligo, Wegener granulomatosis, granulomatous orchitis, autoimmune oophoritis, sarcoidosis, rheumatic carditis, ankylosing spondylitis, Grave's disease, autoimmune thrombocytopenic purpura, psoriasis, psoriatic arthritis, eczema, dermatitis herpetiformis, ulcerative colitis, pancreatic fibrosis, hepatitis, hepatic fibrosis, CD14 mediated sepsis, non-CD14 mediated sepsis, acute and chronic renal disease, irritable bowel syndrome, pyresis, restenosis, cervicitis, stroke and ischemic injury, neural trauma, acute and chronic pain, allergic rhinitis, allergic conjunctivitis, chronic heart failure, congestive heart failure, acute coronary syndrome, cachexia, malaria, leprosy, leishmaniosis, Lyme disease, Reiter's syndrome, acute synovitis, muscle degeneration, bursitis, tendonitis, tenosynovitis, herniated, ruptured, or prolapsed intervertebral disk syndrome, osteopetrosis, rhinosinusitis, thrombosis, silicosis, pulmonary sarcosis, bone resorption diseases, such as osteoporosis, fibromyalgia, AIDS and other viral diseases such as Herpes Zoster, Herpes Simplex I or II, influenza virus and cytomegalovirus, diabetes Type I and II, obesity, insulin resistance and diabetic retinopathy, 22q11.2 deletion syndrome, Angelman syndrome, Canavan disease, celiac disease, Charcot-Marie-Tooth disease, color blindness, Cri du chat, Down syndrome, cystic fibrosis, Duchenne muscular dystrophy, haemophilia, Klinefleter's syndrome, neurofibromatosis, phenylketonuria, Prader-Willi syndrome, sickle cell disease, Tay-Sachs disease, Turner syndrome, urea cycle disorders, thalassemia, otitis, pancreatitis, parotitis, pericarditis, peritonitis, pharyngitis, pleuritis, phlebitis, pneumonitis, uveitis, polymyositis, proctitis, interstitial lung fibrosis, dermatomyositis, atherosclerosis, arteriosclerosis, amyotrophic lateral sclerosis, asociality, varicosis, vaginitis, depression, and Sudden Infant Death Syndrome.

In other embodiments, the methods are directed to treating subjects having cancer. Generally, the compounds of the present invention may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers includes adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), polycythemia vera, lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), germ cell tumor, ovarian germ cell tumor, head and neck cancer, Hodgkin's lymphoma, leukemia, lymphoma, multiple myeloma, hepatocellular carcinoma, hypopharyngeal cancer, intraocular melanoma, ocular cancer, islet cell tumors (endocrine pancreas), renal cancer (e.g., Wilm's Tumor, clear cell renal cell carcinoma), liver cancer, lung cancer (e.g., non-small cell lung cancer and small cell lung cancer), Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, essential thrombocythemia, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, pituitary tumor, plasma cell neoplasm, pleuropulmonary blastoma, prostate cancer, retinoblastoma rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, transitional cell cancer of the renal pelvis and ureter and other urinary organs, urethral cancer, gestational trophoblastic tumor, vaginal cancer and vulvar cancer.

Sarcomas that may be treatable with compounds of the present invention include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue) and mesenchymous or mixed mesodermal tumor (mixed connective tissue types).

In some embodiments, methods of the present invention entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, polycythemia vera, chronic myelocytic leukemia, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, ameningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), squamous cell carcinoma, adenocarcinoma, small cell carcinoma, large cell carcinoma, squamous cell carcinoma, and mesothelioma. Lung cancer can include “scar carcinoma”, bronchoalveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, compounds of the present invention may be used to treat non-metastatic or metastatic lung cancer (e.g., NSCLC, ALK-positive NSCLC, NSCLC harboring ROS1 Rearrangement, Lung Adenocarcinoma, and Squamous Cell Lung Carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell types).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, a compound of the present invention may be used to treat T cell leukemia or T cell lymphoma.

In some embodiments, a compound of the present invention may be used to treat Hodgkin's lymphoma or non-Hodgkin's lymphoma.

In some embodiments, a compound of the present invention may be used to treat myeloid leukemia.

In some embodiments, a compound of the present invention may be used to treat non-small cell lung cancer (NSCLC).

In some embodiments, a compound of the present invention may be used to treat melanoma.

In some embodiments, a compound of the present invention may be used to treat triple-negative breast cancer (TNBC).

In some embodiments, a compound of the present invention may be used to treat nasopharyngeal cancer (NPC).

In some embodiments, a compound of the present invention may be used to treat microsatellite stable colorectal cancer (mssCRC).

In some embodiments, a compound of the present invention may be used to treat thymoma.

In some embodiments, a compound of the present invention may be used to treat carcinoid.

In some embodiments, a compound of the present invention may be used to treat gastrointestinal stromal tumor (GIST).

In some embodiments, the disease or disorder is gout, idiopathic pulmonary fibrosis, silicosis, asbestosis, nonalcoholic steatohepatitis, atherosclerosis, diabetes, diabetic nephropathy, diabetic retinopathy, or diabetic cardiomyopathy.

The compounds of formula (I) and their pharmaceutically acceptable salts and stereoisomers may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which have been unsuccessful, or partially successful but who have become intolerant to the particular treatment. Therapy may also be given as an adjuvant treatment, i.e., to prevent reoccurrence of cancer in patients with no currently detectable disease or after surgical removal of a tumor. Thus, in some embodiments, the compound may be administered to a patient who has received prior therapy, such as chemotherapy, radioimmunotherapy, surgical therapy, immunotherapy, radiation therapy, targeted therapy or any combination thereof.

The methods of the present invention may entail administration of an inventive compound or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day off period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of five days.

### Combination Therapy

The compounds of the present invention and their pharmaceutically acceptable salts, hydrates, solvates, prodrugs, stereoisomers, or tautomers may be used in combination or concurrently with at least one other active agent e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second agent, the first of the two agents is, in some cases, still detectable at effective concentrations at the site of treatment. The sequence and time interval may be determined such that they can act together (e.g., synergistically to provide an increased benefit than if they were administered otherwise). For example, the agents may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of formula (I) or a pharmaceutically acceptable salt, hydrate, solvate, prodrug, stereoisomer, or tautomer thereof in combination with one or more additional therapeutic agents known for use in treating the disease or disorder (e.g., cancer). The dosage of the additional anticancer therapeutic may be the same or even lower than known or recommended doses. See, Hardman et al., eds., Goodman & Gilman's The Pharmacological Basis Of Basis Of Therapeutics, 10th ed., McGraw-Hill, New York, 2001; Physician's Desk Reference 60th ed., 2006. For example, anti-cancer agents that may be used in combination with the inventive compounds are known in the art. See, e.g., U.S. Pat. No. 9,101,622 (Section 5.2 thereof) and U.S. Pat. No. 9,345,705 B2 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immune-modulators, therapeutic antibodies (e.g., mono-specific and bispecific antibodies) and CAR-T therapy.

In some embodiments, the compound of the invention and the additional anticancer therapeutic agent may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more anticancer therapeutics may be administered within the same patient visit.

In some embodiments, the compound of formula (I) and the additional therapeutic agent (e.g., an anti-cancer therapeutic) are cyclically administered. By way of example in the context of cancer treatment, cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, and depending on the particular cancer being treated, the compound of the present invention may be used in combination with at least one other anti-cancer agents such as Paclitaxel (e.g., ovarian cancer, breast cancer, lung cancer, Kaposi sarcoma, cervical cancer, and pancreatic cancer), Topotecan (e.g., ovarian cancer and lung cancer), Irinotecan (e.g., colon cancer, and small cell lung cancer), Etoposide (e.g., testicular cancer, lung cancer, lymphomas, and non-lymphocytic leukemia), Vincristine (e.g., leukemia), Leucovorin (e.g., colon cancer), Altretamine (e.g., ovarian cancer), Daunorubicin (e.g., acute myeloid leukemia (AML), acute lymphocytic leukemia (ALL), chronic myelogenous leukemia (CML), and Kaposi's sarcoma), Trastuzumab (e.g., breast cancer, stomach cancer, and esophageal cancer), Rituximab (e.g., non-Hodgkin's lymphoma), Cetuximab (e.g., colorectal cancer, metastatic non-small cell lung cancer and head and neck cancer), Pertuzumab (e.g., metastatic HER2-positive breast cancer), Alemtuzumab (e.g., chronic lymphocytic leukemia (CLL), cutaneous T-cell lymphoma (CTCL) and T-cell lymphoma), Panitumumab (e.g., colon and rectum cancer), Tamoxifen (e.g., breast cancer), Fulvestrant (e.g., breast cancer), Letrazole (e.g., breast cancer), Exemestane (e.g., breast cancer), Azacytidine (e.g., myelodysplastic syndromes), Mitomycin C (e.g., gastro-intestinal cancers, anal cancers, and breast cancers), Dactinomycin (e.g., Wilms tumor, rhabdomyosarcoma, Ewing's sarcoma, trophoblastic neoplasm, testicular cancer, and ovarian cancer), Erlotinib (e.g., non-small cell lung cancer and pancreatic cancer), Sorafenib (e.g., kidney cancer and liver cancer), Temsirolimus (e.g., kidney cancer), Bortezomib (e.g., multiple myeloma and mantle cell lymphoma), Pegaspargase (e.g., acute lymphoblastic leukemia), Cabometyx (e.g., hepatocellular carcinoma, medullary thyroid cancer, and renal cell carcinoma), Keytruda (e.g., cervical cancer, gastric cancer, hepatocellular carcinoma, Hodgkin lymphoma, melanoma, Merkel cell carcinoma, non-small cell lung cancer, urothelial carcinoma, and squamous cell carcinoma of the head and neck), Nivolumab (e.g., colorectal cancer, hepatocellular carcinoma, melanoma, non-small cell lung cancer, renal cell carcinoma, small cell lung cancer, and urothelial carcinoma), and Regorafenib (e.g., colorectal cancer, gastrointestinal stromal tumor, and hepatocellular carcinoma).

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain a compound of the present invention or a pharmaceutical composition which contains the compound and a pharmaceutically acceptable carrier wherein the compound and the carrier may be disposed in the same or separate containers. The kits or pharmaceutical systems of the invention may also include printed instructions for using the compounds and compositions.

These and other aspects of the present invention will be further appreciated upon consideration of the following Examples, which are intended to illustrate certain particular embodiments of the invention but are not intended to limit its scope, as defined by the claims.

### EXAMPLES

### Example 1. Synthesis of 3-(3-bromo-2,5-dioxo-2,5-dihydro-1H-pyrrol-1-yl)-piperidine-2,6-dione

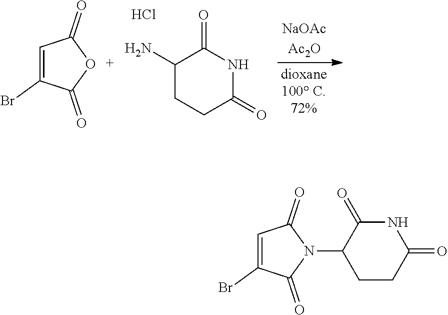 [SMILES: O=C1C=C(Br)C(=O)O1 ; NC1CCC(=O)NC1=O ; Cl ; O=C1CCC(N2C(=O)C=C(Br)C2=O)C(=O)N1]

In a 40-mL vial, 3-aminopiperidine-2,6-dione hydrochloride (1.0 g, 6.08 mmol) was dissolved in dioxane (14 mL, 0.45 M). Bromomaleic anhydride (621 μL, 6.68 mmol) was added, and the reaction mixture was stirred at 80° C. for 1 hour. Sodium acetate (550 mg, 6.68 mmol) was added, and the reaction mixture was stirred at 80° C. for 5 hours. Acetic anhydride (632 μL, 6.68 mmol) was added dropwise, and the reaction mixture was stirred at 100° C. for 15 hours. Upon cooling to room temperature (rt), the reaction mixture was concentrated in vacuo. The crude product was dissolved in CH₂Cl₂ and carefully washed with satd. aq. NaHCO₃ and water. The organic layers were collected, dried over Na₂SO₄, and filtered. Concentration in vacuo provided a crude oil, which was dissolved in water and acetonitrile, frozen, and lyophilized. The title compound was obtained as a light brown solid (1.25 g, 72% yield), which was used without further purification.

### Example 2: Synthesis of 3-(2,5-dioxo-3-((3-(2-((3-(trifluoromethyl)phenethyl)amino)propan-2-yl)phenyl)amino)-2,5-dihydro-1H-pyrrol-1-yl)piperidine-2,6-dione (65)

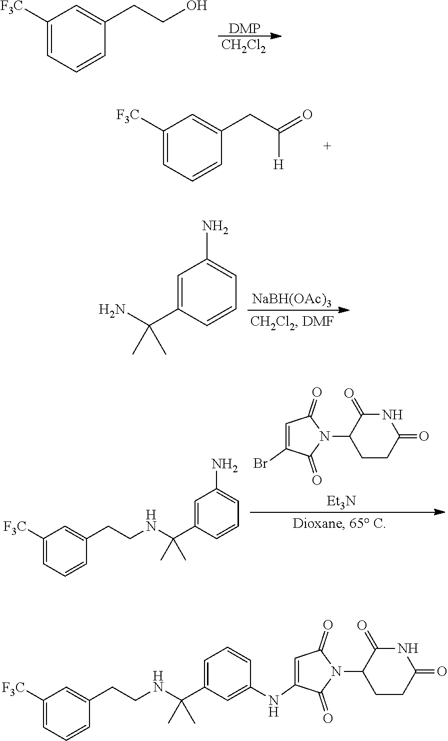

### 2-(3-(trifluoromethyl)phenyl)-acetaldehyde

To a solution of 2-(3-(trifluoromethyl)-phenyl)ethan-1-ol (338 mg, 1.78 mmol) in CH₂Cl₂ (0.2 M) was added Dess-Martin periodinane (DMP; 1.13 g, 2.67 mmol). The reaction mixture was stirred at rt for 3 hours. Upon complete conversion of starting material, the reaction mixture was diluted with CH₂Cl₂, washed with satd. aq. Na₂S₂O₃, H₂O, dried over Na₂SO₄, filtered over Celite®, and concentrated in vacuo. The title compound was obtained in quantitative yield (335 mg), and the crude material was taken forward without further purification.

### 3-(2-((3-(trifluoromethyl)phenethyl)amino)propan-2-yl)aniline

To a solution of 2-(3-(trifluoromethyl)phenyl)-acetaldehyde (136 mg, 0.723 mmol) and 3-(2-aminopropan-2-yl)aniline (109 mg, 0.723 mmol) in 1:1 CH₂Cl₂/DMF (8 mL) was added sodium triacetoxyborohydride (317 mg, 2.17 mmol). The reaction mixture was stirred at rt for 14 hours. The reaction was diluted with CH₂Cl₂, washed with satd. aq. Na₂CO₃, dried over Na₂SO₄, filtered, and concentrated in vacuo. Purification by silica flash chromatography yielded the title compound (200 mg). LC-MS m/z: (pos) 323.12 ([M+H]⁺).

### 3-(2,5-dioxo-3-((3-(2-((3-(trifluoromethyl)phenethyl)amino)propan-2-yl)phenyl)amino)-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (110 mg, 0.414 mmol) and 3-(2-((3-(trifluoromethyl)phenethyl)amino)propan-2-yl)aniline (200 mg, 0.62 mmol) in dioxane (4 mL) was added triethylamine (173 μL, 1.24 mmol). The reaction was stirred at 65° C. for 16 hours. Upon cooling to rt, the reaction mixture was filtered over a short plug of silica and Celite®, and concentrated in vacuo. Purification by preparative thin layer chromatography (prep TLC) with 1:20 1.75 N NH₃ in MeOH/CH₂Cl₂ yielded the title compound as a yellow solid (11.8 mg). LC-MS m/z: (pos) 529.19 ([M+H]⁺).

### Example 3: Synthesis of 3-(3-((3-(2-((3-chloro-4-(trifluoromethyl)phenethyl)amino)propan-2-yl)phenyl)amino)-2,5-dioxo-2,5-dihydro-1H-pyrrol-1-yl)piperidine-2,6-dione (66)

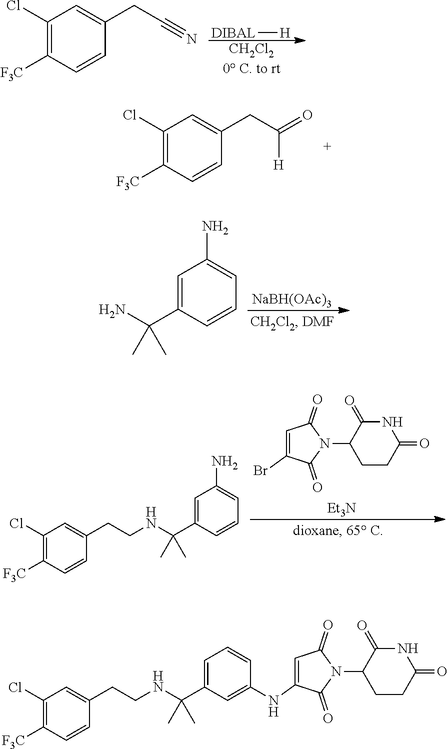

### 2-(3-chloro-4-(trifluoromethyl)phenyl)acetaldehyde

2-(3-chloro-4-(trifluoromethyl)-phenyl)acetonitrile (245.8 mg, 1.12 mmol) was dissolved in CH₂Cl₂ (0.1 M) and cooled to 0° C. Diisobutylaluminium hydride (1.34 mmol) was added dropwise as a 1M solution in CH₂Cl₂. The reaction mixture was stirred at 0° C. for 2 hours. Upon warming to rt, the reaction mixture was extracted with CH₂Cl₂, washed with a satd. aq. solution of Rochelle salt, dried over Na₂SO₄, filtered, and concentrated in vacuo. The crude title compound was obtained (218.6 mg), and was taken forward without further purification. LC-MS m/z: (pos) 223.99 ([M+H]⁺).

### 3-(2-((3-chloro-4-(trifluoromethyl)phenethyl)amino)propan-2-yl)aniline

To a solution of 2-(3-chloro-4-(trifluoromethyl)phenyl)acetaldehyde (218.6 mg, 0.982 mmol) and 3-(2-aminopropan-2-yl)aniline (295 mg, 1.96 mmol) in 1:1 CH₂Cl₂/DMF (10 mL) was added sodium triacetoxyborohydride (624 mg, 2.95 mmol) and stirred at rt for 14 hours. The reaction was diluted with CH₂Cl₂, washed with satd. aq. Na₂CO₃, dried over Na₂SO₄, filtered, and concentrated in vacuo. Purification by silica flash chromatography yielded the title compound (214.4 mg). LC-MS m/z: (pos) 357.03 ([M+H]⁺).

### 3-(3-((3-(2-((3-chloro-4-(trifluoromethyl)phenethyl)amino)propan-2-yl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (123.2 mg, 0.429 mmol) and 3-(2-((3-chloro-4-(trifluoromethyl)phenethyl)amino)propan-2-yl)aniline (214.4 mg, 0.601 mmol) in dioxane (4 mL) was added triethylamine (180 μL, 1.29 mmol). The reaction was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound as a yellow solid. LC-MS m/z: (pos) 563.11 ([M+H]⁺).

### Example 4: Synthesis of 3-(3-((3-(2-((3-chloro-4-(trifluoromethyl)phenethyl)(methyl)amino)propan-2-yl)phenyl)-amino)-2,5-dioxo-2,5-dihydro-1H-pyrrol-1-yl)piperidine-2,6-dione (67)

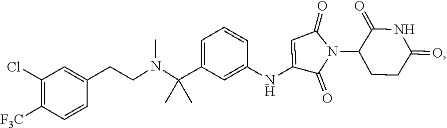 [SMILES: CN(CCc1ccc(C(F)(F)F)c(Cl)c1)C(C)(C)c1cccc(NC2=CC(=O)N(C3CCC(=O)NC3=O)C2=O)c1]

Crude 3-(3-((3-(2-((3-chloro-4-(trifluoromethyl)phenethyl)amino)propan-2-yl)phenyl)amino)-2,5-dioxo-2,5-dihydro-1H-pyrrol-1-yl)piperidine-2,6-dione (200 mg, 0.355 mmol) was dissolved in CH₂Cl₂ (4 mL) and was cooled to 0° C. Iodomethane (110 μL, 1.78 mmol) and N,N-diisopropylethylamine (DIPEA; 620 μL, 3.55 mmol) were added at 0° C. The reaction mixture was stirred at 0° C. for 30 min, then stirred at rt for 4 hours. To avoid bis-methylation, the reaction mixture was extracted with CH₂Cl₂, washed with water, dried over Na₂SO₄, filtered, and concentrated in vacuo. Purification by prep TLC with 1:20 1.75 N NH₃ in MeOH/CH₂Cl₂ yielded the title compound as a yellow solid. LC-MS m/z: (pos) 577.50 ([M+H]⁺).

### Example 5: Synthesis of 3-(3-((-(((3-chloro-4-(trifluoromethyl)phenethyl)(methyl)amino)methyl)phenyl)amino)-2,5-dioxo-2,5-dihydro-1H-pyrrol-1-yl)piperidine-2,6-dione (48)

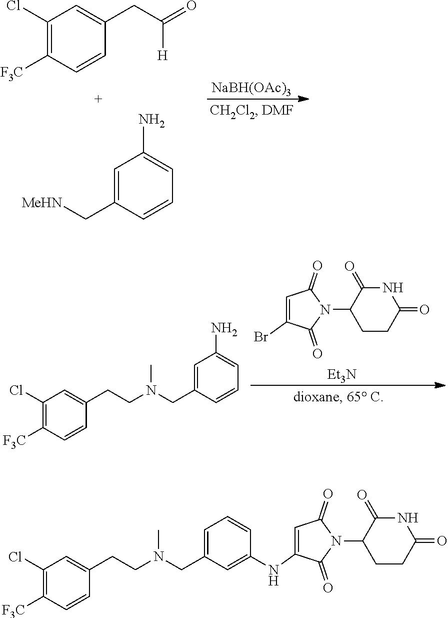

### 3-(((3-chloro-4-(trifluoromethyl)phenethyl)(methyl)amino)methyl)aniline

To a solution of 2-(3-chloro-4-(trifluoromethyl)phenyl)acetaldehyde (230.9 mg, 1.04 mmol) and 3-((methylamino)methyl)aniline (283.3 mg, 2.08 mmol) was added sodium triacetoxyborohydride (661.3 mg, 3.12 mmol) and stirred at rt for 14 hours. The reaction mixture was extracted with CH₂Cl₂, washed with satd. aq. Na₂CO₃, dried over Na₂SO₄, filtered, and concentrated in vacuo. Purification by silica flash chromatography yielded the title compound (338.7 mg). LC-MS m/z: (pos) 343.05 ([M+H]⁺).

### 3-(3-((-(((3-chloro-4-(trifluoromethyl)phenethyl)(methyl)amino)methyl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (189 mg, 0.659 mmol) and 3-(((3-chloro-4-(trifluoromethyl)phenethyl)(methyl)amino)-methyl)aniline (338.7 mg, 0.988 mmol) in dioxane (8 mL) was added triethylamine (276 μL, 1.98 mmol). The reaction mixture was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound. LC-MS m/z: (pos) 549.13 ([M+H]⁺).

### Example 6: Synthesis of 3-(3-((-(((3-chloro-4-(trifluoromethyl)phenethyl)(methyl)-amino)methyl)phenyl)amino)-2,5-dioxo-2,5-dihydro-1H-pyrrol-1-yl)piperidine-2,6-dione (47)

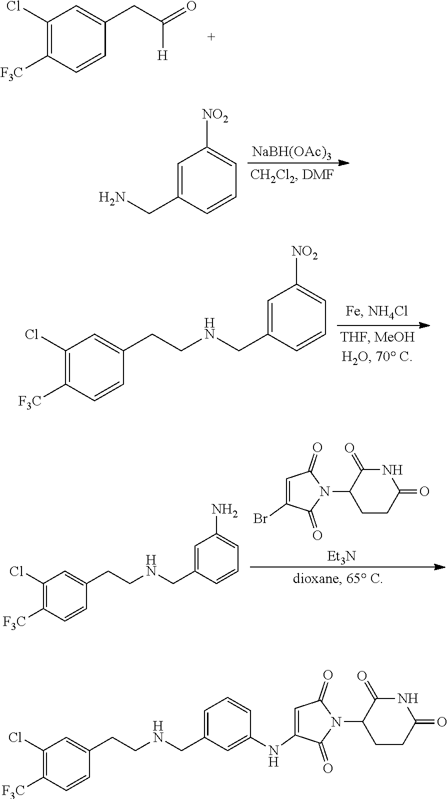

### 3-(((3-chloro-4-(trifluoromethyl)phenethyl)amino)methyl)aniline

To a solution of 2-(3-chloro-4-(trifluoromethyl)phenyl)acetaldehyde (290.2 mg, 1.30 mmol) and (3-nitrophenyl)methanamine (270.5 mg, 1.43 mmol) was added sodium triacetoxyborohydride (661.3 mg, 3.12 mmol) and stirred at rt for 14 hours. The reaction mixture was extracted with CH₂Cl₂, washed with satd. aq. Na₂CO₃, dried over Na₂SO₄, filtered, and concentrated in vacuo to give the crude nitro-intermediate, which was taken forward directly to nitro reduction. LC-MS m/z: (pos) 358.97 ([M+H]⁺).

The crude nitro-intermediate was dissolved in a solution of THF (4 mL) and MeOH (1 mL). Iron powder (218 mg, 3.9 mmol) was added, followed by 2 mL of satd. aq. NH₄Cl. The reaction mixture was stirred at 70° C. for 3 hours. Upon cooling to rt, the reaction mixture was extracted with CH₂Cl₂, washed with H₂O, dried over Na₂SO₄, filtered over Celite®, and concentrated in vacuo. The title compound was obtained (232.1 mg) and taken forward without purification. LC-MS m/z: (pos) 329.01 ([M+H]⁺).

### 3-(3-((3-(((3-chloro-4-(trifluoromethyl)phenethyl)amino)methyl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (145 mg, 0.505 mmol) and 3-(((3-chloro-4-(trifluoromethyl)phenethyl)amino)-methyl)aniline (232.1 mg, 0.706 mmol) in dioxane (8 mL) was added triethylamine (211 μL, 1.52 mmol). The reaction mixture was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound. LC-MS m/z: (pos) 535.09 ([M+H]⁺).

### Example 7: Synthesis of 3-(3-((3-(1-((3-chloro-4-(trifluoromethyl)phenethyl)-amino)propyl)phenyl)amino)-2,5-dioxo-2,5-dihydro-1H-pyrrol-1-yl)piperidine-2,6-dione (60)

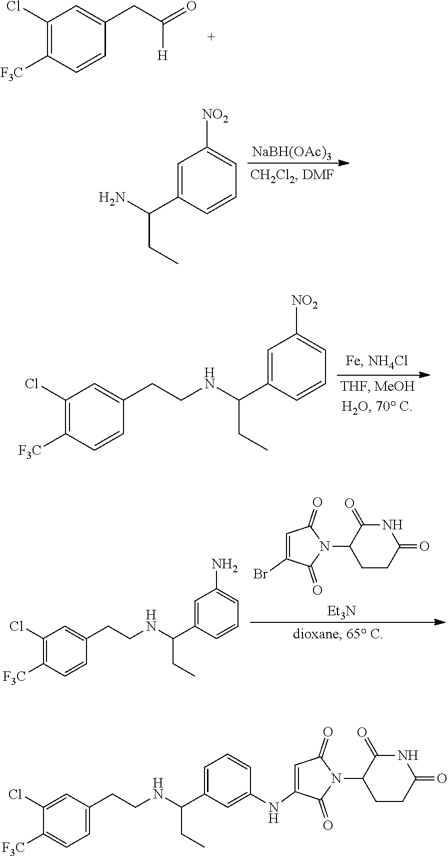

### N-(3-chloro-4-(trifluoromethyl)phenethyl)-1-(3-nitrophenyl)propan-1-amine

To a solution of 2-(3-chloro-4-(trifluoromethyl)phenyl)acetaldehyde (224.3 mg, 1.01 mmol) and 1-(3-nitrophenyl)propan-1-amine (151.3 mg, 0.84 mmol) was added sodium triacetoxyborohydride (534 mg, 2.52 mmol) and stirred at rt for 14 hours. The reaction mixture was extracted with CH₂Cl₂, washed with satd. aq. Na₂CO₃, dried over Na₂SO₄, filtered, and concentrated in vacuo to yield the crude title compound (85.4 mg). LC-MS m/z: (pos) 387.28 ([M+H]⁺).

### 3-(1-((3-chloro-4-(trifluoromethyl)phenethyl)amino)propyl)aniline

Crude N-(3-chloro-4-(trifluoromethyl)phenethyl)-1-(3-nitrophenyl)propan-1-amine (85.4 mg, 0.221 mmol) was dissolved in a solution of THF (4 mL) and MeOH (1 mL). Iron powder (37 mg, 0.66 mmol) was added, followed by 2 mL satd. aq. NH₄Cl. The reaction mixture was stirred at 70° C. for 4 hours. Upon cooling to rt, the reaction mixture was extracted with CH₂Cl₂, washed with H₂O, dried over Na₂SO₄, filtered over Celite®, and concentrated in vacuo. The crude title compound was obtained and taken forward without purification. LC-MS m/z: (pos) 357.26 ([M+H]⁺).

### 3-(3-((3-(1-((3-chloro-4-(trifluoromethyl)phenethyl)amino)propyl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (71 mg, 0.248 mmol) and 3-(1-((3-chloro-4-(trifluoromethyl)phenethyl)-amino)propyl)aniline (115 mg, 0.322 mmol) in dioxane (4 mL) was added triethylamine (104 μL, 0.744 mmol). The reaction was stirred at 65° C. for 15 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound as a yellow solid. LC-MS m/z: (pos) 563.46 ([M+H]⁺).

### Example 8: Synthesis of 3-(3-((3-(1-((3-chloro-4-(trifluoromethyl)phenethyl)-amino)propyl)phenyl)amino)-2,5-dioxo-2,5-dihydro-1H-pyrrol-1-yl)piperidine-2,6-dione (36)

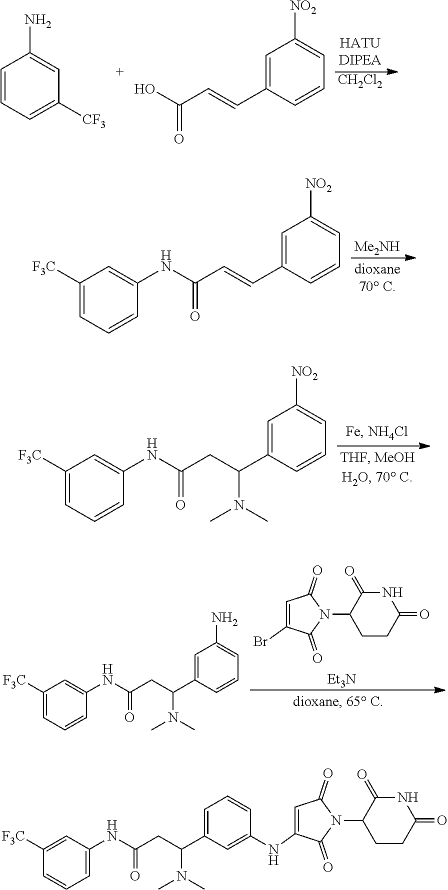

### (E)-3-(3-nitrophenyl)-N-(3-(trifluoromethyl)phenyl)acrylamide

To a solution of (E)-3-(3-nitrophenyl)acrylic acid (1.04 g, 5.4 mmol) in CH₂Cl₂ (20 mL) was added hexafluorophosphate azabenzotriazole tetramethyl uronium (HATU; 2.26 g, 5.94 mmol), DIPEA (2.82 mL, 16.2 mmol), and 3-(trifluoromethyl)aniline (870 mg, 5.4 mmol) sequentially. The reaction mixture was stirred at rt for 4 hours. Upon complete conversion of (E)-3-(3-nitrophenyl)acrylic acid the reaction mixture was concentrated in vacuo. Purification by silica flash chromatography yielded the title compound as a light yellow solid in quantitative yield (1.81 g). LC-MS m/z: (pos) 336.98 ([M+H]⁺).

### 3-(dimethylamino)-3-(3-nitrophenyl)-N-(3-(trifluoromethyl)phenyl)propanamide

To a solution of (E)-3-(3-nitrophenyl)-N-(3-(trifluoromethyl)phenyl)acrylamide (443 mg, 1.31 mmol) in dioxane (5 mL) was added dimethylamine as a 2 M solution in THF (3.3 mL, 6.6 mmol). The reaction mixture was stirred at 70° C. for 16 hours. Partial conversion was observed, and upon cooling to rt, the reaction mixture was concentrated in vacuo. Purification by silica flash chromatography yield the title compound (200 mg) and recovered (E)-3-(3-nitrophenyl)-N-(3-(trifluoromethyl)phenyl)acrylamide (185 mg). LC-MS m/z: (pos) 382.33 ([M+H]⁺).

### 3-(3-aminophenyl)-3-(dimethylamino)-N-(3-(trifluoromethyl)phenyl)propanamide

3-(dimethylamino)-3-(3-nitrophenyl)-N-(3-(trifluoromethyl)phenyl)propanamide (200 mg, 0.524 mmol) was dissolved in a solution of THF (4 mL) and MeOH (1 mL). Iron powder (88 mg, 1.57 mmol) was added, followed by 2 mL satd. aq. NH₄Cl. The reaction mixture was stirred at 70° C. for 4 hours. Upon cooling to rt, the reaction mixture was extracted with CH₂Cl₂, washed with H₂O, dried over Na₂SO₄, filtered over Celite®, and concentrated in vacuo. The title compound (147 mg) was obtained as a brown oil, and crude material was taken forward without purification. LC-MS m/z: (pos) 352.31 ([M+H]⁺).

### 3-(dimethylamino)-3-(3-((1-(2,6-dioxopiperidin-3-yl)-2,5-dioxo-2,5-dihydro-1H-pyrrol-3-yl)amino)phenyl)-N-(3-(trifluoromethyl)phenyl)propanamide

To a solution of 3-(3-bromo-2,5-dioxo-2,5-dihydro-1H-pyrrol-1-yl)piperidine-2,6-dione (92 mg, 0.322 mmol) and 3-(3-aminophenyl)-3-(dimethylamino)-N-(3-(trifluoromethyl)phenyl)propanamide (147 mg, 0.418 mmol) in dioxane (4 mL) was added triethylamine (135 μL, 0.966 mmol). The reaction was stirred at 65° C. for 15 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound. LC-MS m/z: (pos) 558.21 ([M+H]⁺).

### Example 9: Synthesis of 3-(azetidin-1-yl)-3-(3-((1-(2,6-dioxopiperidin-3-yl)-2,5-dioxo-2,5-dihydro-1H-pyrrol-3-yl)amino)phenyl)-N-(3-(trifluoromethyl)phenyl)propanamide (38)

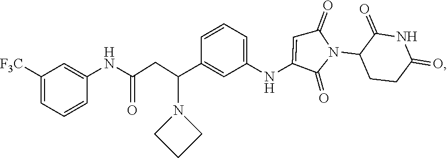 [SMILES: O=C1CCC(N2C(=O)C=C(Nc3cccc(C(CC(=O)Nc4cccc(C(F)(F)F)c4)N4CCC4)c3)C2=O)C(=O)N1]

Compound 38 was prepared in a similar manner as compound 36. LC-MS m/z: (pos) 570.30 ([M+H]⁺).

### Example 10: Synthesis of N-(3-chloro-4-methylphenyl)-3-(3-((1-(2,6-dioxopiperidin-3-yl)-2,5-dioxo-2,5-dihydro-1H-pyrrol-3-yl)amino)phenyl)-4-methylpentanamide (30)

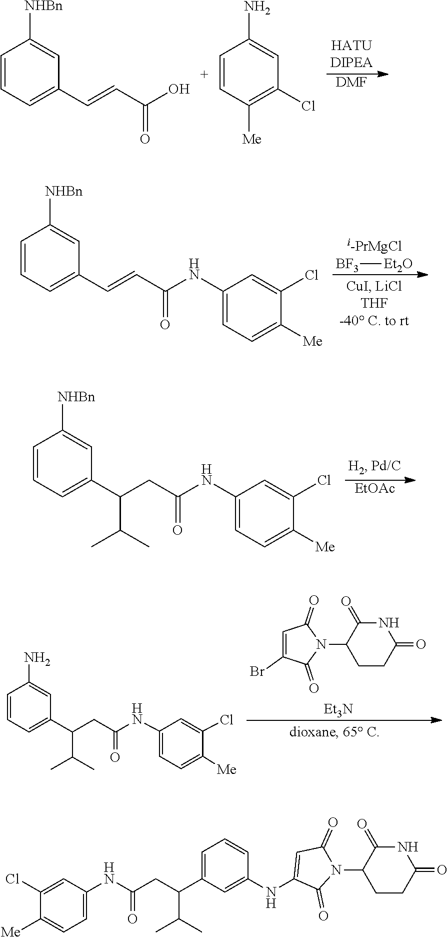

### (E)-3-(3-(benzylamino)phenyl)-N-(3-chloro-4-methylphenyl)acrylamide

To a solution of (E)-3-(3-(benzylamino)phenyl)acrylic acid (1.025 g, 4.05 mmol) in DMF (10 mL) was added HATU (1.85 g, 4.86 mmol), DIPEA (2.12 mL, 12.15 mmol), and 3-chloro-4-methylaniline (540 μL, 0.446 mmol), sequentially. The reaction mixture was stirred at rt for 3 hours. Upon complete conversion of (E)-3-(3-(benzylamino)phenyl)acrylic acid, the reaction mixture was extracted with EtOAc, washed with H₂O 2×, brine, dried over Na₂SO₄, filtered, and concentrated in vacuo. Purification by silica flash chromatography (0-80% EtOAc/hexanes) yielded the title compound as a yellow solid (1.217 g, 80% yield). LC-MS m/z: (pos) 377.19 ([M+H]⁺).

### 3-(3-(benzylamino)phenyl)-N-(3-chloro-4-methylphenyl)-4-methylpentanamide

(E)-3-(3-(benzylamino)phenyl)-N-(3-chloro-4-methylphenyl)acrylamide (221 mg, 0.586 mmol) in THF (6 mL) was put under a nitrogen atmosphere. Copper (I) iodide (11.2 mg, 0.059 mmol) and lithium chloride (5 mg, 0.117 mmol) was added, and the reaction mixture was stirred under N₂ for 1 hour at rt. The reaction mixture was then cooled to −40° C., and boron trifluoride etherate (73 μL, 0.586 mmol) was added dropwise over 10 minutes. Isopropylmagnesium chloride (2.35 mmol) was added dropwise over 1 hour as a 2 M solution in THF. The reaction mixture was stirred at −40° C. for 1 hour and then slowly warmed to rt. The reaction mixture was carefully quenched with H₂O, extracted with EtOAc, washed with H₂O, brine, dried over Na₂SO₄, filtered, and concentrated in vacuo. Purification by silica flash chromatography yielded the title compound as a brown oil (62.3 mg, 25% yield). LC-MS m/z: (pos) 421.25 ([M+H]⁺), 443.24 ([M+Na]⁺).

### 3-(3-aminophenyl)-N-(3-chloro-4-methylphenyl)-4-methylpentanamide

To a solution of 3-(3-(benzylamino)phenyl)-N-(3-chloro-4-methylphenyl)-4-methylpentanamide (62.3 mg, 0.15 mmol) in EtOAc (6 mL) was added palladium on carbon (0.015 mmol), followed by the attachment of a H₂ balloon. The reaction mixture was stirred under H₂ for 5 hours. Upon complete conversion, the reaction mixture was filtered over Celite® to remove the Pd catalyst, washed with EtOAc, and concentrated in vacuo to yield the crude title compound, which was used without further purification. LC-MS m/z: (pos) 331.13 ([M+H]⁺).

### N-(3-chloro-4-methylphenyl)-3-(3-((1-(2,6-dioxopiperidin-3-yl)-2,5-dioxo-2,5-dihydro-1H-pyrrol-3-yl)amino)phenyl)-4-methylpentanamide

To a solution of 3-(3-bromo-2,5-dioxo-2,5-dihydro-1H-pyrrol-1-yl)piperidine-2,6-dione (60 mg, 0.209 mmol) and 3-(3-aminophenyl)-N-(3-chloro-4-methylphenyl)-4-methylpentanamide (97 mg, 0.293 mmol) in dioxane (4 mL) was added triethylamine (60 μL, 0.418 mmol). The reaction was stirred at 65° C. for 15 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound. ¹H NMR (500 MHz, DMSO-d₆) δ 11.05 (s, 1H), 9.89 (s, 1H), 9.75 (s, 1H), 7.66 (d, J=2.0 Hz, 1H), 7.28 (t, J=8.1 Hz, 1H), 7.25-7.15 (m, 4H), 6.96 (d, J=7.6 Hz, 1H), 5.63 (s, 1H), 4.96 (dd, J=13.0, 5.4 Hz, 1H), 2.98 (dt, J=9.8, 6.1 Hz, 1H), 2.86 (ddd, J=17.1, 13.8, 5.3 Hz, 1H), 2.78 (dd, J=14.8, 5.5 Hz, 1H), 2.66-2.53 (m, 2H), 2.45 (dd, J=13.3, 4.5 Hz, 1H), 2.22 (s, 3H), 2.01-1.95 (m, 1H), 1.92-1.83 (m, 1H), 0.92 (d, J=6.6 Hz, 3H), 0.75 (d, J=6.7 Hz, 3H). LC-MS m/z: (pos) 537.14 ([M+H]⁺).

### Example 11: Synthesis of 3-(3-((1-(2,6-dioxopiperidin-3-yl)-2,5-dioxo-2,5-dihydro-1H-pyrrol-3-yl)amino)phenyl)-3-phenyl-N-(3-(trifluoromethyl)phenyl)propanamide (40)

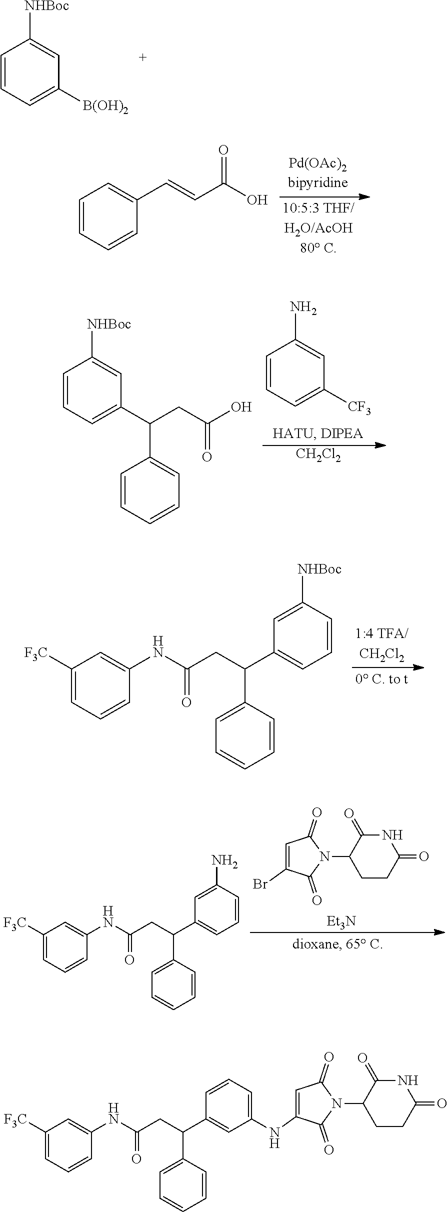

### 3-(3-((tert-butoxycarbonyl)amino)phenyl)-3-phenylpropanoic acid

To a solution of (3-((tert-butoxycarbonyl)amino)phenyl)boronic acid (516 mg, 2.18 mmol) in THF (5 mL) was added Pd(OAc)₂ (12.2 mg, 0.055 mmol) and bipyridine (17.2 mg, 0.11 mmol). Cinnamic acid (161.4 mg, 1.09 mmol) was then added, followed by H₂O (2.5 mL) and AcOH (800 μL). The reaction mixture was stirred at 80° C. for 16 hours. Upon cooling to rt, the reaction mixture was extracted with EtOAc, washed with H₂O, dried over Na₂SO₄, filtered over Celite®, and concentrated in vacuo. Purification by silica flash chromatography (0-60% EtOAc/CH₂Cl₂ yielded the title compound (126.8 mg, 34% yield). LC-MS m/z: (pos) 364.33 ([M+Na]⁺).

### tert-Butyl (3-(3-oxo-1-phenyl-3-((3-(trifluoromethyl)phenyl)amino)propyl)phenyl)carbam-ate

To a solution of 3-(3-((tert-butoxycarbonyl)amino)phenyl)-3-phenylpropanoic acid (126.8 mg, 0.371 mmol) in 10:1 CH₂Cl₂/DMF (3.3 mL) was added HATU (170 mg, 0.446), DIPEA (194 μL, 1.11 mmol) and 3-(trifluoromethyl)aniline (72 mg, 0.446 mmol), sequentially. The reaction mixture was stirred at rt for 3 hours. Upon complete conversion, the reaction mixture was extracted with CH₂Cl₂, washed with H₂O, dried over Na₂SO₄, and filtered, and concentrated in vacuo. Purification by silica flash chromatography yielded the title compound (47.5 mg). LC-MS m/z: (pos) 507.15 ([M+Na]⁺).

### 3-(3-aminophenyl)-3-phenyl-N-(3-(trifluoromethyl)phenyl)propanamide

tert-Butyl (3-(3-oxo-1-phenyl-3-((3-(trifluoromethyl)phenyl)-amino)propyl)phenyl)carbamate (47.5 mg, 0.098 mmol) was dissolved in CH₂Cl₂ (2.4 mL) and cooled to 0° C. Trifluoroacetic acid (600 μL) was added. The reaction mixture was stirred at 0° C. for 1 hour and then at rt for 2 hours. Upon complete conversion, the reaction mixture was concentrated in vacuo to give the title compound in quantitative yield, and was taken forward without purification. LC-MS m/z: (pos) 385.10 ([M+H]⁺).

### 3-(3-((1-(2,6-dioxopiperidin-3-yl)-2,5-dioxo-2,5-dihydro-1H-pyrrol-3-yl)amino)phenyl)-3-phenyl-N-(3-(trifluoromethyl)phenyl)propanamide

To a solution of 3-(3-bromo-2,5-dioxo-2,5-dihydro-1H-pyrrol-1-yl)piperidine-2,6-dione (26 mg, 0.089 mmol) and 3-(3-aminophenyl)-3-phenyl-N-(3-(trifluoromethyl)phenyl)propanamide (37.7 mg, 0.098 mmol) in dioxane (3 mL) was added triethylamine (37 μL, 0.267 mmol). The reaction was stirred at 65° C. for 15 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound as a yellow solid (8.8 mg). LC-MS m/z: (pos) 591.24 ([M+H]⁺), 613.18 ([M+Na]⁺).

### Example 12: Synthesis of N-(3-chloro-4-(trifluoromethyl)phenyl)-4-(dimethylamino)-3-(3-((1-(2,6-dioxopiperidin-3-yl)-2,5-dioxo-2,5-dihydro-1H-pyrrol-3-yl)amino)phenyl)butanamide (73)

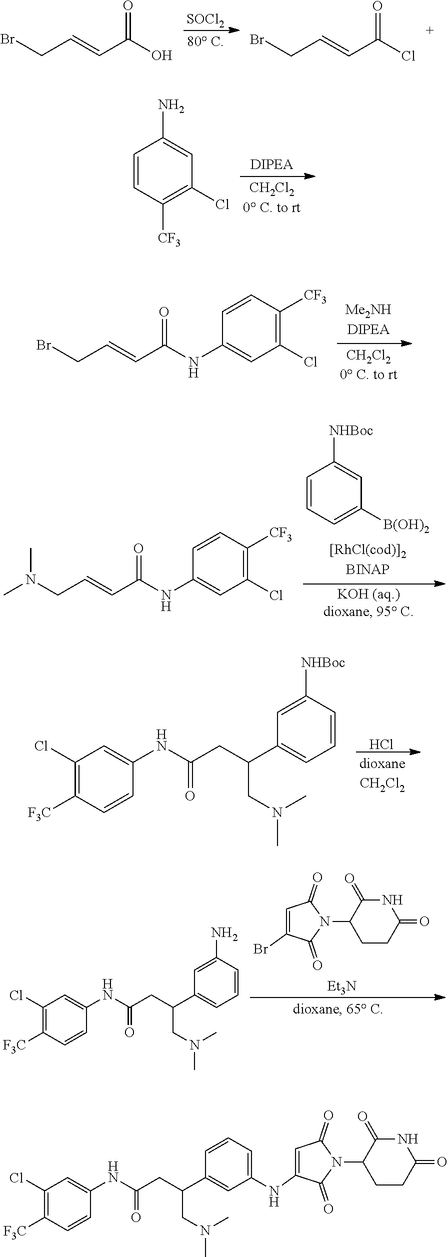

### (E)-4-bromobut-2-enoyl chloride

(E)-4-bromobut-2-enoic acid (1 g, 6.06 mmol) was dissolved in thionyl chloride (1 M) and stirred at 80° C. for 3 hours. Upon cooling to rt, the reaction mixture was concentrated in vacuo to yield the title compound which was stored as a 0.1 M solution in CH₂Cl₂.

### (E)-4-bromo-N-(3-chloro-4-(trifluoromethyl)phenyl)but-2-enamide

A solution of (E)-4-bromobut-2-enoyl chloride (1.84 mmol) in CH₂Cl₂ (10 mL) was cooled to 0° C. A solution of 3-chloro-4-(trifluoromethyl)aniline (300 mg, 1.54 mmol) and DIPEA (400 μL, 2.30 mmol) in 5 mL CH₂Cl₂ was added dropwise to (E)-4-bromobut-2-enoyl chloride at 0° C. The reaction mixture was stirred at 0° C., then warmed to rt over 1 hour. The reaction mixture was then diluted with CH₂Cl₂, washed with H₂O, dried over Na₂SO₄, filtered, and concentrated in vacuo to yield the title compound (276.7 mg), which was taken forward without purification. LC-MS m/z: (pos) 342.11 ([M+H]⁺).

### tert-Butyl (3-(4-((3-chloro-4-(trifluoromethyl)phenyl)amino)-1-(dimethylamino)-4-oxo-butan-2-yl)phenyl)carbamate

(E)-4-bromo-N-(3-chloro-4-(trifluoromethyl)phenyl)but-2-enamide (276.7 mg, 0.808 mmol) was dissolved in CH₂Cl₂ (2 mL) and cooled to 0° C. DIPEA (281 μL, 1.62 mmol) was added, followed by dimethylamine (1.21 mmol) as a 2 M solution in THF. The reaction mixture was stirred at 0° C. for 1 hour, then warmed to rt for 1 hour. The reaction mixture was extracted with CH₂Cl₂, washed with H₂O, dried over Na₂SO₄, filtered, and concentrated in vacuo to give the crude dimethylamino intermediate, which was taken forward without purification. LC-MS m/z: (pos) 307.25 ([M+H]⁺).

To a solution of (3-((tert-butoxycarbonyl)amino)phenyl)boronic acid (575 mg, 2.42 mmol) in dioxane (8.1 mL) under nitrogen gas was added cyclooctadiene rhodium chloride dimer (19.9 mg, 0.0404 mmol) and 2,2′-bis(diphenylphosphino)-1,1′-binaphthyl (BINAP; 101 mg, 0.162 mmol). The crude intermediate (247.8 mg, 0.808 mmol) was then added, followed by potassium hydroxide (1.62 mmol) as a 3.8 M solution in H₂O. The reaction mixture was purged with N₂ gas, and stirred at 95° C. for 12 hours. Upon cooling to rt, the reaction mixture was extracted with EtOAc, washed with H₂O, dried over Na₂SO₄, filtered over Celite®, and concentrated in vacuo. Purification by silica flash chromatography yielded the title compound (237.4 mg, 59% yield). LC-MS m/z: (pos) 500.53 ([M+H]⁺).

### 3-(3-aminophenyl)-N-(3-chloro-4-(trifluoromethyl)phenyl)-4-(dimethylamino)butanamide

To a solution of tert-butyl (3-(4-((3-chloro-4-(trifluoromethyl)phenyl)amino)-1-(dimethylamino)-4-oxo-butan-2-yl)phenyl)carbamate (237.4 mg, 0.475 mmol) in CH₂Cl₂ (5 mL) was added hydrochloric acid (0.95 mmol) as a 4 M solution in dioxane. The reaction mixture was stirred at rt for 10 hours. Upon complete conversion, the reaction mixture was concentrated in vacuo to yield the title compound (188 mg), which was taken forward without purification. LC-MS m/z: (pos) 400.38 ([M+H]⁺).

### N-(3-chloro-4-(trifluoromethyl)phenyl)-4-(dimethylamino)-3-(3-((1-(2,6-dioxopiperidin-3-yl)-2,5-dioxo-2,5-dihydro-1H-pyrrol-3-yl)amino)phenyl)butanamide

To a solution of 3-(3-bromo-2,5-dioxo-2,5-dihydro-1H-pyrrol-1-yl)piperidine-2,6-dione (52 mg, 0.181 mmol) and 3-(3-aminophenyl)-N-(3-chloro-4-(trifluoromethyl)phenyl)-4-(dimethylamino)butanamide (94 mg, 0.235 mmol) in dioxane (4 mL) was added triethylamine (76 μL, 0.543 mmol). The reaction mixture was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound as a yellow solid. LC-MS m/z: (pos) 606.52 ([M+H]⁺).

### Example 13: Synthesis of 3-(3-((3-(1-((3-chloro-4-methylphenyl)amino)-4-methylpentan-3-yl)phenyl)amino)-2,5-dioxo-2,5-dihydro-1H-pyrrol-1-yl)piperidine-2,6-dione (78)

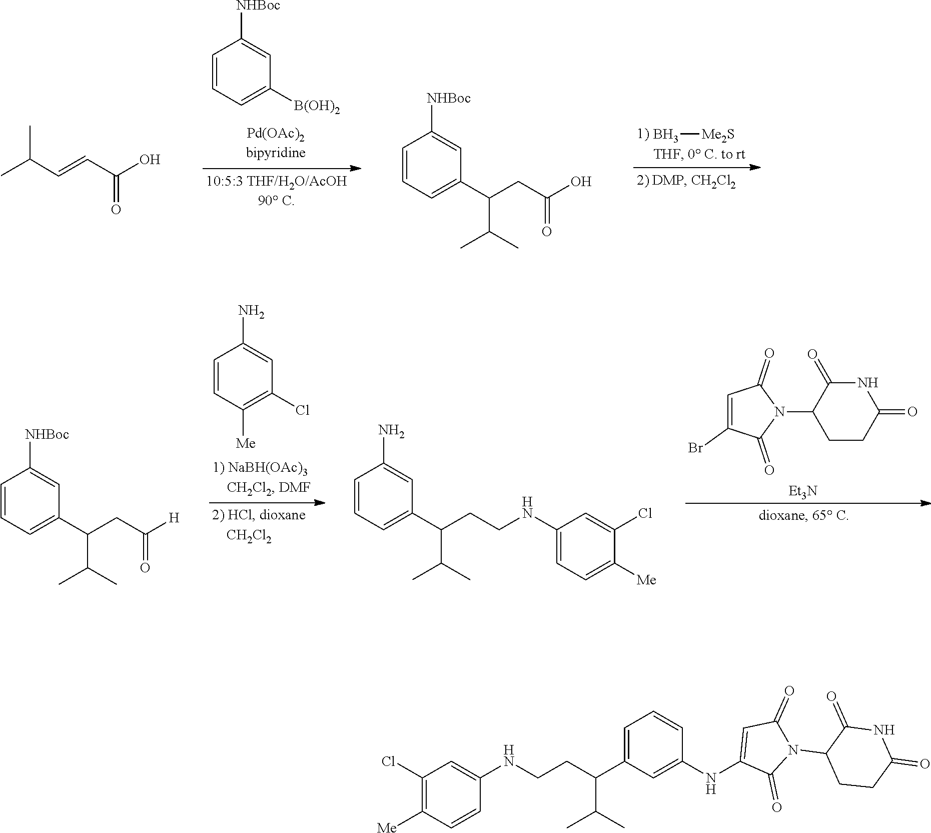

### 3-(3-((tert-butoxycarbonyl)amino)phenyl)-4-methylpentanoic acid

To a solution of (3-((tert-butoxycarbonyl)amino)phenyl)boronic acid (2 g, 8.43 mmol) in THF (8 mL) was added Pd(OAc)₂ (95 mg, 0.422 mmol) and bipyridine (132 mg, 0.844 mmol). (E)-4-methylpent-2-enoic acid (503 μL, 4.22 mmol) was then added, followed by H₂O (4 mL) and AcOH (2 mL). The reaction mixture was stirred 90° C. for 16 hours. Upon cooling to rt, the reaction mixture was extracted with CH₂Cl₂, washed with H₂O, dried over Na₂SO₄, filtered over Celite®, and concentrated in vacuo. Purification by silica flash chromatography (0-20% EtOAc/CH₂Cl₂) yielded the title compound as a beige solid (520.1 mg, 40% yield). LC-MS m/z: (pos) 330.14 ([M+Na]⁺).

### tert-Butyl (3-(4-methyl-1-oxopentan-3-yl)phenyl)carbamate

3-(3-((tert-butoxycarbonyl)amino)phenyl)-4-methylpentanoic acid (168.7 mg, 0.549 mmol) was dissolved in THF (6 mL) and cooled to 0° C. Borane dimethylsulfide (63 μL, 0.659 mmol) was added dropwise at 0° C. The reaction mixture was stirred at 0° C. for 1 hour and then at rt for 3 hours. Upon complete conversion, the reaction was quenched carefully by dropwise addition of MeOH and concentrated in vacuo to give the alcohol intermediate as a light yellow oil in quantitative yield, which was taken forward without purification LC-MS m/z: (pos) 316.10 ([M+Na]⁺).

The crude alcohol intermediate (161.1 mg, 0.549 mmol) was then dissolved in CH₂Cl₂ (8 mL). DMP (350 mg, 0.824 mmol) was added and the reaction mixture stirred at rt for 4 hours. The reaction mixture was extracted with CH₂Cl₂, washed with satd. aq. Na₂S₂O₃, dried over Na₂SO₄, filtered, and concentrated in vacuo to give the crude title compound, which was taken forward without purification. LC-MS m/z: (pos) 314.33 ([M+Na]⁺).

### N-(3-(3-aminophenyl)-4-methylpentyl)-3-chloro-4-methylaniline

tert-Butyl (3-(4-methyl-1-oxopentan-3-yl)phenyl)carbamate (208 mg, 0.714 mmol) and 3-chloro-4-methylaniline (111 mg, 0.785 mmol) were dissolved in 1:1 solution of CH₂Cl₂/DMF (6 mL). Sodium triacetoxyborohydride (303 mg, 1.43 mmol) was added, and the reaction mixture was stirred for 16 hours. The reaction mixture was extracted with CH₂Cl₂, washed with satd. aq. NaHCO₃, H₂O, dried over Na₂SO₄, filtered, and concentrated in vacuo. The crude product was then dissolved in CH₂Cl₂ (4 mL) and 2 mL of 4 M HCl in dioxane was added and stirred at rt for 12 hours. Upon complete conversion, the reaction mixture was concentrated in vacuo to give the title compound as a brown oil (206.4 mg, 91% yield). LC-MS m/z: (pos) 316.28 ([M+H]⁺).

### 3-(3-((3-(1-((3-chloro-4-methylphenyl)amino)-4-methylpentan-3-yl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (156 mg, 0.543 mmol) and N-(3-(3-aminophenyl)-4-methylpentyl)-3-chloro-4-methylaniline (206.4 mg, 0.651 mmol) in dioxane (6 mL) was added triethylamine (230 μL, 1.63 mmol). The reaction was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound. LC-MS m/z: (pos) 523.53 ([M+H]⁺).

### Example 14: Synthesis of 3-(3-((3-(4-((3-chloro-4-methylphenyl)amino)-2-methylbutan-2-yl)phenyl)amino)-2,5-dioxo-2,5-dihydro-1H-pyrrol-1-yl)piperidine-2,6-dione (79)

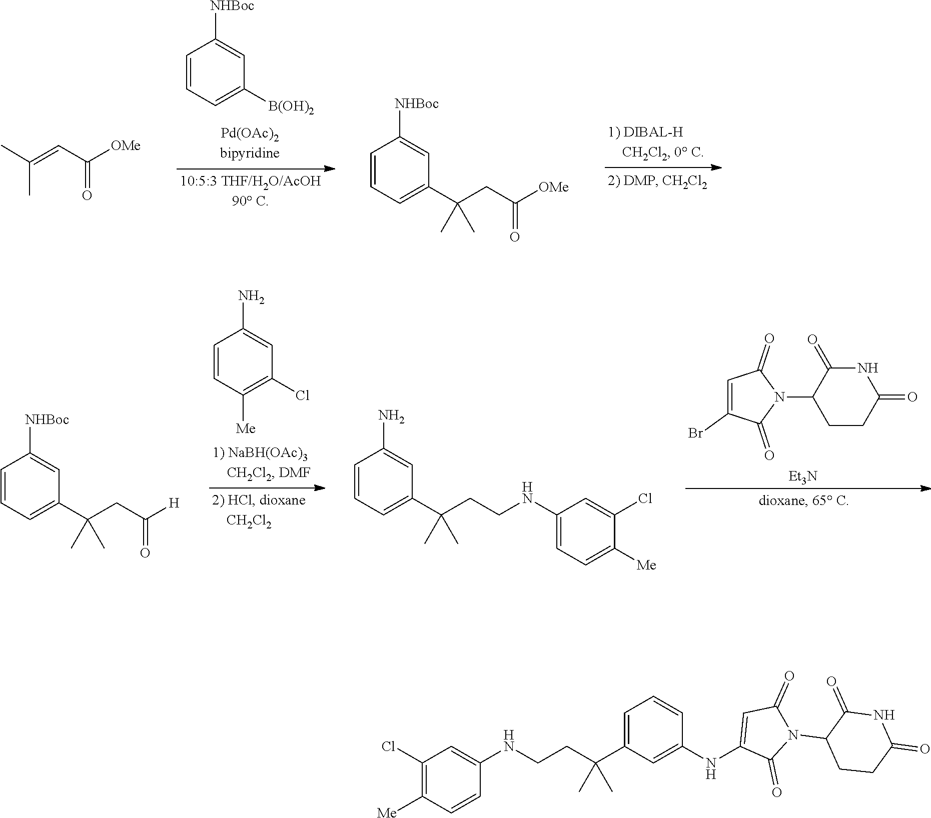

### Methyl 3-(3-((tert-butoxycarbonyl)amino)phenyl)-3-methylbutanoate

To a solution of (3-((tert-butoxycarbonyl)amino)phenyl)boronic acid (2 g, 8.43 mmol) in THF (8 mL) was added Pd(OAc)₂ (95 mg, 0.422 mmol) and bipyridine (132 mg, 0.844 mmol). Methyl 3-methylbut-2-enoate (481 mg, 4.22 mmol) was then added, followed by H₂O (4 mL) and AcOH (2 mL). The reaction mixture was stirred 90° C. for 16 hours. Upon cooling to rt, the reaction mixture was extracted with CH₂Cl₂, washed with H₂O, dried over Na₂SO₄, filtered over Celite®, and concentrated in vacuo. Purification by silica flash chromatography (0-50% EtOAc/CH₂Cl₂) yielded the title compound as a light yellow oil (524.1 mg, 40% yield). LC-MS m/z: (pos) 330.08 ([M+Na]⁺).

### tert-Butyl (3-(2-methyl-4-oxobutan-2-yl)phenyl)carbamate

Methyl 3-(3-((tert-butoxycarbonyl)amino)phenyl)-3-methylbutanoate (524.1 mg, 1.71 mmol) was dissolved in CH₂Cl₂ (0.3 M) and cooled to 0° C. Diisobutylaluminum hydride (2.57 mmol) was added dropwise to the reaction mixture as a 1 M solution in CH₂Cl₂. The reaction mixture was stirred at 0° C. for 3 hours. Upon warming to rt, the reaction mixture was extracted with CH₂Cl₂, washed with a satd. aq. solution of Rochelle salt, dried over Na₂SO₄, filtered, and concentrated in vacuo to give the crude tert-Butyl (3-(4-hydroxy-2-methylbutan-2-yl)phenyl)carbamate as a light yellow oil (467 mg, 98% yield). The crude material was taken forward directly to oxidation. LC-MS m/z: (pos) 302.12 ([M+Na]⁺).

tert-Butyl (3-(4-hydroxy-2-methylbutan-2-yl)phenyl)carbamate (467 mg, 1.67 mmol) was then dissolved in CH₂Cl₂ (10 mL). DMP (1.06 g, 2.51 mmol) was added and the reaction mixture was stirred at rt for 5 hours. The reaction mixture was extracted with CH₂Cl₂, washed with satd. aq. Na₂S₂O₃, dried over Na₂SO₄, filtered, and concentrated in vacuo to give the crude title compound as a yellow orange solid (358.3 mg, 77% yield), which was used without further purification. LC-MS m/z: (pos) 300.12 ([M+Na]⁺).

### N-(3-(3-aminophenyl)-3-methylbutyl)-3-chloro-4-methylaniline

tert-Butyl (3-(2-methyl-4-oxobutan-2-yl)phenyl)carbamate (163.9 mg, 0.591 mmol) and 3-chloro-4-methylaniline (100 mg, 0.709 mmol) were dissolved in a4:1 solution of CH₂Cl₂/DMF (8 mL). Sodium triacetoxyborohydride (251 mg, 1.18 mmol) was added, and the reaction mixture was stirred for 16 hours. The reaction mixture was extracted with CH₂Cl₂, washed with satd. aq. NaHCO₃, H₂O, dried over Na₂SO₄, filtered, and concentrated in vacuo. Purification by silica flash chromatography (0-40% EtOAc/hexanes) yielded tert-butyl (3-(4-((3-chloro-4-methylphenyl)amino)-2-methylbutan-2-yl)phenyl)carbamate (87.6 mg, 37% yield). LC-MS m/z: (pos) 403.44 ([M+H]⁺).

tert-Butyl (3-(4-((3-chloro-4-methylphenyl)amino)-2-methylbutan-2-yl)phenyl)carbamate (87.6 mg, 0.217 mmol) was then dissolved in CH₂Cl₂ (4 mL). 6 mL of 4 M HCl in dioxane was then added and stirred at rt for 18 hours. Upon complete conversion, the reaction mixture was concentrated in vacuo to give the title compound as an orange solid (65.7 mg) in quantitative yield. LC-MS m/z: (pos) 303.30 ([M+H]⁺).

### 3-(3-((3-(4-((3-chloro-4-methylphenyl)amino)-2-methylbutan-2-yl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (52 mg, 0.181 mmol) and N-(3-(3-aminophenyl)-3-methylbutyl)-3-chloro-4-methylaniline (65.7 mg, 0.217 mmol) in dioxane (4 mL) was added triethylamine (80 μL, 0.543 mmol). The reaction was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound as a yellow solid. LC-MS m/z: (pos) 509.55 ([M+H]⁺).

### Example 15: Synthesis of 3-(3-((3-(4-((3-chloro-4-(trifluoromethyl)phenyl)amino)-2-methylbutan-2-yl)phenyl)amino)-2,5-dioxo-2,5-dihydro-1H-pyrrol-1-yl)piperidine-2,6-dione (80)

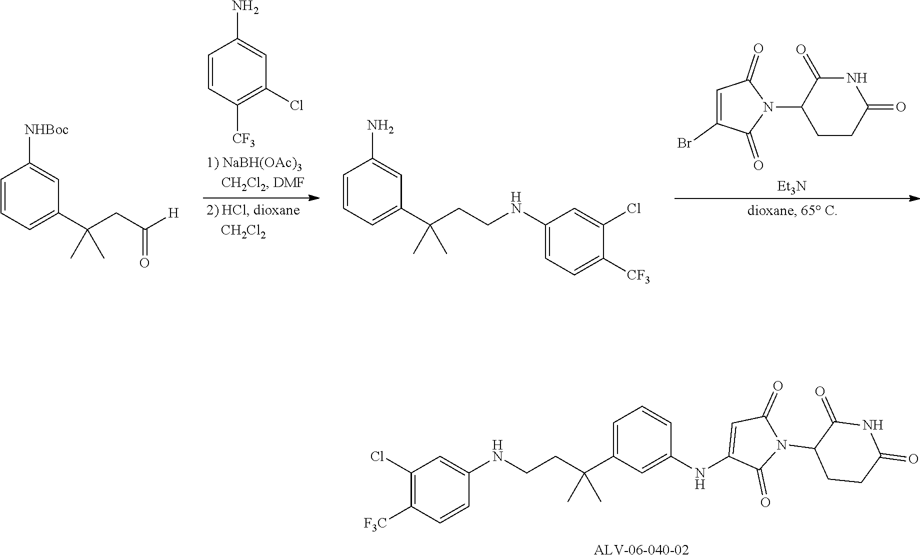 [SMILES: "Et3N" ; CC(C)(C)OC(=O)Nc1cccc(C(C)(C)CC=O)c1 ; Nc1ccc(C(F)(F)F)c(Cl)c1 ; CC(C)(CCNc1ccc(C(F)(F)F)c(Cl)c1)c1cccc(N)c1 ; O=C1CCC(N2C(=O)C=C(Br)C2=O)C(=O)N1 ; CC(C)(CCNc1ccc(C(F)(F)F)c(Cl)c1)c1cccc(NC2=CC(=O)N(C3CCC(=O)NC3=O)C2=O)c1]

### N-(3-(3-aminophenyl)-3-methylbutyl)-3-chloro-4-(trifluoromethyl)aniline

tert-Butyl (3-(2-methyl-4-oxobutan-2-yl)phenyl)carbamate (194.4 mg, 0.701 mmol) and 3-chloro-4-(trifluoromethyl)aniline (165 mg, 0.841 mmol) were dissolved in a4:1 solution of CH₂Cl₂/DMF (8 mL). Sodium triacetoxyborohydride (297 mg, 1.4 mmol) was added, and the reaction mixture was stirred for 16 hours. The reaction mixture was extracted with CH₂Cl₂, washed with satd. aq. NaHCO₃, H₂O, dried over Na₂SO₄, filtered, and concentrated by rotary evaporation. Purification by silica flash chromatography (0-40% EtOAc/hexanes) yielded tert-butyl (3-(4-((3-chloro-4-methylphenyl)amino)-2-methylbutan-2-yl)phenyl)carbamate (140.9 mg, 44% yield). LC-MS m/z: (pos) 457.47 ([M+H]⁺).

tert-butyl (3-(4-((3-chloro-4-methylphenyl)amino)-2-methylbutan-2-yl)phenyl)carbamate (140.9 mg, 0.308 mmol) was then dissolved in CH₂Cl₂ (4 mL). Six (6) mL of 4 M HCl in dioxane was then added and stirred at rt for 18 hours. Upon complete conversion, the reaction mixture was concentrated in vacuo to give the title compound as a light yellow solid (79.6 mg, 72% yield). LC-MS m/z: (pos) 357.26 ([M+H]⁺).

### 3-(3-((3-(4-((3-chloro-4-(trifluoromethyl)phenyl)amino)-2-methylbutan-2-yl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (50 mg, 0.172 mmol) and N-(3-(3-aminophenyl)-3-methylbutyl)-3-chloro-4-(trifluoromethyl)aniline (79.6 mg, 0.223 mmol) in dioxane (4 mL) was added triethylamine (72 μL, 0.516 mmol). The reaction was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound as a yellow solid. LC-MS m/z: (pos) 563.46 ([M+H]⁺).

### Example 16: Synthesis of 3-(3-((3-(1-((naphthalen-2-ylmethyl)amino)ethyl)phenyl)amino)-2,5-dioxo-2,5-dihydro-1H-pyrrol-1-yl)piperidine-2,6-dione (33)

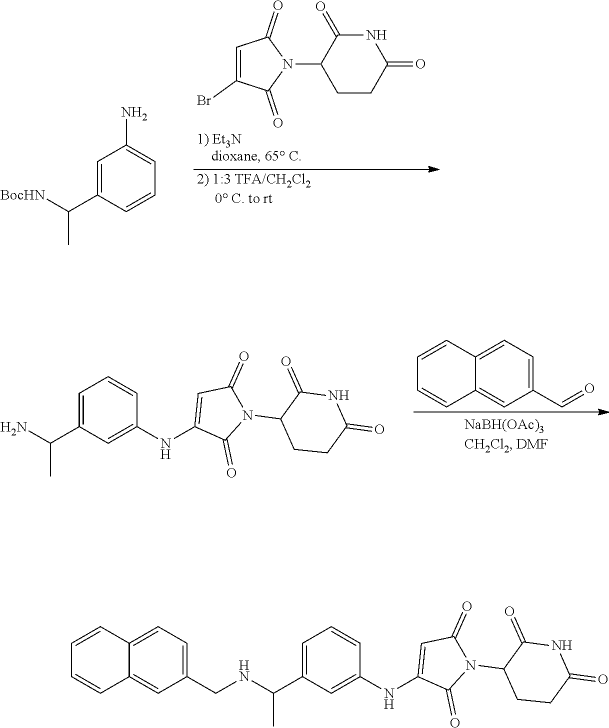

### 3-(3-((3-(1-aminoethyl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (315 mg, 1.097 mmol) and tert-butyl (1-(3-aminophenyl)ethyl)carbamate (363 mg, 1.54 mmol) in dioxane (10 mL) was added triethylamine (460 μL, 3.29 mmol). The reaction was stirred at 65° C. for 16 hours. Upon cooling to rt, the reaction mixture was filtered over a short plug of silica and Celite®, and concentrated in vacuo. Purification by silica flash chromatography yielded tert-butyl (1-(3-((1-(2,6-dioxopiperidin-3-yl)-2,5-dioxo-2,5-dihydro-1H-pyrrol-3-yl)amino)phenyl)ethyl)carbamate as a brown oil. LC-MS m/z: (pos) 465.42 ([M+Na]⁺).

tert-Butyl (1-(3-((1-(2,6-dioxopiperidin-3-yl)-2,5-dioxo-2,5-dihydro-1H-pyrrol-3-yl)amino)-phenyl)ethyl)carbamate was then dissolved in CH₂Cl₂ (6 mL) and cooled to 0° C. Trifluoroacetic acid (2 mL) was added dropwise at 0° C. The reaction mixture was stirred at 0° C. for 10 minutes and then warmed to rt for 1 hour. Upon complete conversion, the reaction mixture was concentrated in vacuo. Precipitation from ether yielded the title compound as a light brown solid (300 mg, 80% over 2 steps). LC-MS m/z: (pos) 343.34 ([M+H]⁺).

### 3-(3-((3-(1-((naphthalen-2-ylmethyl)amino)ethyl)phenyl)amino)-2,5-dioxo-2,5-dihydro-1H-pyrrol-1-yl)piperidine-2,6-dione

2-Naphthaldehyde (36 mg, 0.23 mmol) and 3-(3-((3-(1-aminoethyl)phenyl)amino)-2,5-dioxo-2,5-dihydro-1H-pyrrol-1-yl)piperidine-2,6-dione (60 mg, 0.175 mmol) were dissolved in a 1:1 solution of CH₂Cl₂/DMF (4 mL). Sodium triacetoxyborohydride (111 mg, 0.53 mmol) was added, and the reaction mixture was stirred for 14 hours. The reaction mixture was extracted with CH₂Cl₂, washed with satd. aq. NaHCO₃, dried over Na₂SO₄, filtered, and concentrated in vacuo. Purification by prep TLC with 1:20 1.75 N NH₃ in MeOH/CH₂Cl₂ yielded the title compound as a yellow solid. LC-MS m/z: (pos) 483.59 ([M+H]⁺).

### Example 17: Synthesis of 3-(3-((3-(2-(((1H-indazol-6-yl)methyl)amino)propan-2-yl)phenyl)amino)-2,5-dioxo-2,5-dihydro-1H-pyrrol-1-yl)piperidine-2,6-dione (2)

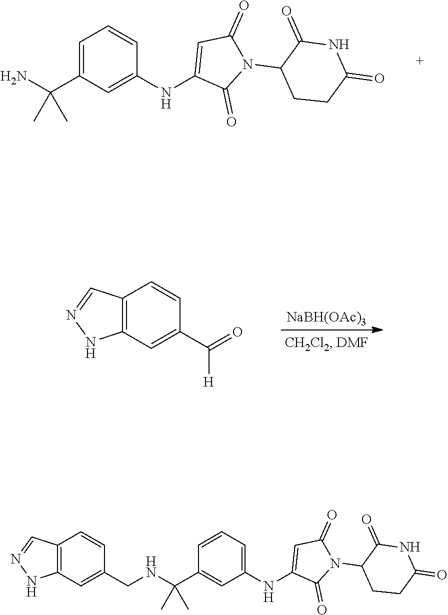

1H-Indazole-6-carbaldehyde (33 mg, 0.23 mmol) and 3-(3-((3-(2-aminopropan-2-yl)phenyl)amino)-2,5-dioxo-2,5-dihydro-1H-pyrrol-1-yl)piperidine-2,6-dione (60 mg, 0.175 mmol) were dissolved in 1:1 CH₂Cl₂/DMF (4 mL). Sodium triacetoxyborohydride (111 mg, 0.53 mmol) was added, and the reaction mixture was stirred for 14 hours. The reaction mixture was extracted with CH₂Cl₂, washed with satd. aq. NaHCO₃, dried over Na₂SO₄, filtered, and concentrated in vacuo. Purification by prep TLC with 1:20 1.75 N NH₃ in MeOH/CH₂Cl₂ yielded the title compound as a yellow solid. LC-MS m/z: (pos) 487.20 ([M+H]⁺).

### Example 18: Synthesis of 3-(3-((3-((methyl(3-(trifluoromethyl)benzyl)amino)-methyl)phenyl)amino)-2,5-dioxo-2,5-dihydro-1H-pyrrol-1-yl)piperidine-2,6-dione (34)

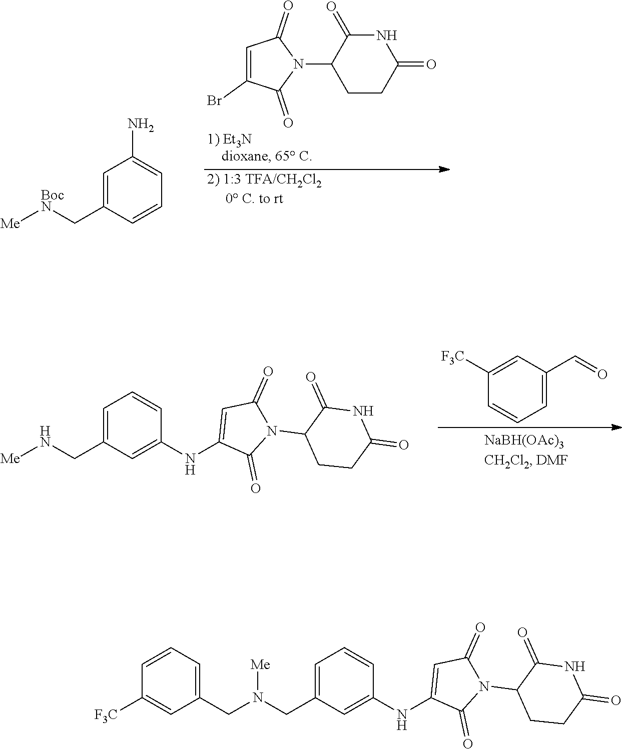

### 3-(3-((3-((methylamino)methyl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (344 mg, 1.2 mmol) and tert-butyl (3-aminobenzyl)(methyl)carbamate (425 mg, 1.8 mmol) in dioxane (10 mL) was added triethylamine (502 μL, 3.6 mmol). The reaction was stirred at 65° C. for 16 hours. Upon cooling to rt, the reaction mixture was filtered over a short plug of silica and Celite®, and concentrated in vacuo. Purification by silica flash chromatography (0-80% EtOAc/CH₂Cl₂) yielded tert-butyl (3-((1-(2,6-dioxopiperidin-3-yl)-2,5-dioxo-2,5-dihydro-1H-pyrrol-3-yl)amino)benzyl)-(methyl)carbamate as a red brown oil (253.3 mg, 48% yield). LC-MS m/z: (pos) 465.48 ([M+Na]⁺).

tert-Butyl (3-((1-(2,6-dioxopiperidin-3-yl)-2,5-dioxo-2,5-dihydro-1H-pyrrol-3-yl)amino)benzyl)-(methyl)carbamate was then dissolved in CH₂Cl₂ (6 mL) and cooled to 0° C. Trifluoroacetic acid (2 mL) was added dropwise at 0° C. The reaction mixture was stirred at 0° C. for 10 minutes and then warmed to rt for 1 hour. Upon complete conversion, the reaction mixture was concentrated in vacuo provided title compound as a red brown solid (200 mg) in quantitative yield. LC-MS m/z: (pos) 343.28 ([M+H]⁺).

### 3-(3-((3-((methyl(3-(trifluoromethyl)benzyl)amino)methyl)phenyl)amino)-2,5-dioxo-2,5-dihydro-1H-pyrrol-1-yl)piperidine-2,6-dione

3-(trifluoromethyl)benzaldehyde (46 mg, 0.263 mmol) and 3-(3-((3-((methylamino)methyl)phenyl)amino)-2,5-dioxo-2,5-dihydro-1H-pyrrol-1-yl)piperidine-2,6-dione (60 mg, 0.175 mmol) were dissolved in a solution of CH₂Cl₂ (3 mL) and DMF (500 μL). Sodium triacetoxyborohydride (74 mg, 0.35 mmol) was added, and the reaction mixture was stirred for 14 hours. The reaction mixture was extracted with CH₂Cl₂, washed with satd. aq. NaHCO₃, dried over Na₂SO₄, filtered, and concentrated in vacuo. Purification by prep TLC with 1:20 1.75 N NH₃ in MeOH/CH₂Cl₂ yielded the title compound as a yellow solid. LC-MS m/z: (pos) 501.18 ([M+H]⁺).

### Example 19: Synthesis of 3-(3-((3-(1-((3-chloro-4-methylbenzyl)amino)-2-methylpropan-2-yl)phenyl)amino)-2,5-dioxo-2,5-dihydro-1H-pyrrol-1-yl)piperidine-2,6-dione (81)

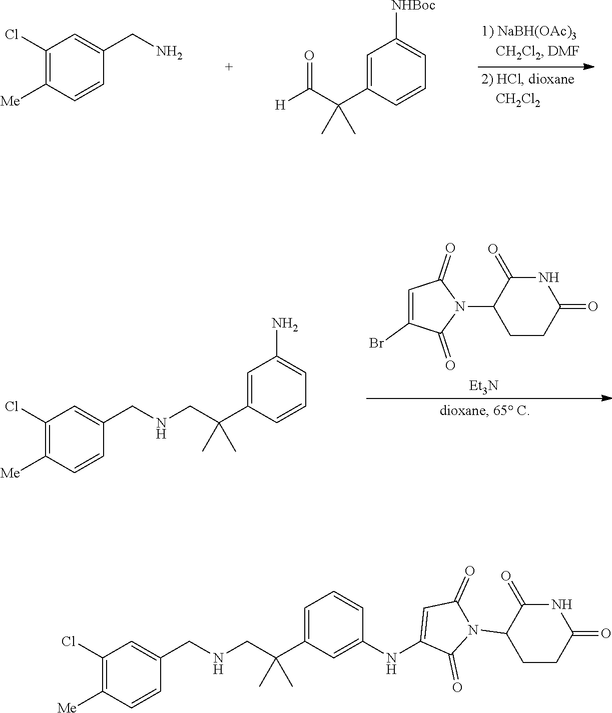

### 3-(1-((3-chloro-4-methylbenzyl)amino)-2-methylpropan-2-yl)aniline

(3-chloro-4-methylphenyl)methanamine (62 mg, 0.398 mmol) and tert-butyl (3-(2-methyl-1-oxopropan-2-yl)phenyl)carbamate (95.5 mg, 0.362 mmol) were dissolved in a solution of CH₂Cl₂ (4 mL) and DMF (2 mL). Sodium triacetoxyborohydride (154 mg, 0.724 mmol) was added, and the reaction mixture was stirred for 14 hours. The reaction mixture was extracted with CH₂Cl₂, washed with satd. aq. NaHCO₃, dried over Na₂SO₄, filtered, and concentrated in vacuo to give tert-butyl (3-(1-((3-chloro-4-methylbenzyl)amino)-2-methylpropan-2-yl)phenyl)carbamate, which was taken forward without purification. LC-MS m/z: (pos) 403.44 ([M+H]⁺).

tert-Butyl (3-(1-((3-chloro-4-methylbenzyl)amino)-2-methylpropan-2-yl)phenyl)carbamate was dissolved in 3 mL CH₂Cl₂. HCl in dioxane (4 M, 1 mL) was then added, and the reaction mixture was stirred at rt for 12 hours. Upon complete conversion, the reaction mixture was concentrated in vacuo to give the title compound. LC-MS m/z: (pos) 303.30 ([M+H]⁺).

### 3-(3-((3-(1-((3-chloro-4-methylbenzyl)amino)-2-methylpropan-2-yl)phenyl)amino)-2,5-dioxo-2,5-dihydro-1H-pyrrol-1-yl)piperidine-2,6-dione

To a solution of 3-(3-bromo-2,5-dioxo-2,5-dihydro-1H-pyrrol-1-yl)piperidine-2,6-dione (80 mg, 0.278 mmol) and 3-(1-((3-chloro-4-methylbenzyl)amino)-2-methylpropan-2-yl)aniline (109.6 mg, 0.362 mmol) in dioxane (4 mL) was added triethylamine (120 μL, 0.834 mmol). The reaction was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound as a yellow solid. LC-MS m/z: (pos) 509.49 ([M+H]⁺).

### Example 20: Synthesis of N-(1-(3-((1-(2,6-dioxopiperidin-3-yl)-2,5-dioxo-2,5-dihydro-1H-pyrrol-3-yl)amino)phenyl)-2,2,2-trifluoroethyl)-2-(3-(trifluoromethyl)phenyl)acetamide (71)

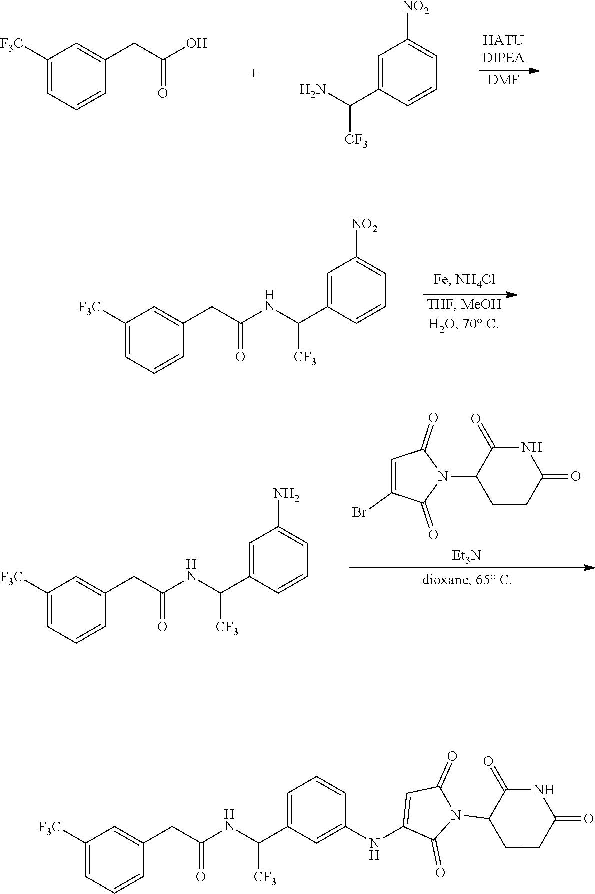

### N-(2,2,2-trifluoro-1-(3-nitrophenyl)ethyl)-2-(3-(trifluoromethyl)phenyl)acetamide

To a solution of 2-(3-(trifluoromethyl)phenyl)acetic acid (117 mg, 0.571 mmol) in DMF (2 mL) was sequentially added HATU (235 mg, 0.62 mmol), DIPEA (250 μL, 1.43 mmol), and 2,2,2-trifluoro-1-(3-nitrophenyl)ethan-1-amine (105 mg, 0.476 mmol). The reaction mixture was stirred at rt for 3 hours. Upon complete conversion, the reaction mixture was extracted with CH₂Cl₂, washed with H₂O ×2, dried over Na₂SO₄, filtered, and concentrated in vacuo to give the crude title compound, which was taken forward without purification. LC-MS m/z: (pos) 407.04 ([M+H]⁺).

### N-(1-(3-aminophenyl)-2,2,2-trifluoroethyl)-2-(3-(trifluoromethyl)phenyl)acetamide

Crude N-(2,2,2-trifluoro-1-(3-nitrophenyl)ethyl)-2-(3-(trifluoromethyl)phenyl)acetamide (193 mg, 0.476 mmol) was dissolved in a solution of THF (4 mL) and MeOH (1 mL). Iron powder (80 mg, 1.43 mmol) was added, followed by 2 mL satd. aq. NH₄Cl. The reaction mixture was stirred at 70° C. for 3 hours. Upon cooling to rt, the reaction mixture was extracted with CH₂Cl₂, washed with H₂O, dried over Na₂SO₄, filtered over Celite®, and concentrated in vacuo. Purification by silica flash chromatography (0-80% EtOAc/CH₂Cl₂) yielded the title compound as a yellow solid (134.2 mg, 75% yield). LC-MS m/z: (pos) 377.08 ([M+H]⁺).

### N-(1-(3-((1-(2,6-dioxopiperidin-3-yl)-2,5-dioxo-2,5-dihydro-1H-pyrrol-3-yl)amino)phenyl)-2,2,2-trifluoroethyl)-2-(3-(trifluoromethyl)phenyl)acetamide

To a solution of 3-(3-bromo-2,5-dioxo-2,5-dihydro-1H-pyrrol-1-yl)piperidine-2,6-dione (39.4 mg, 0.137 mmol) and N-(1-(3-aminophenyl)-2,2,2-trifluoroethyl)-2-(3-(trifluoromethyl)phenyl)acetamide (67.4 mg, 0.178 mmol) in dioxane (3 mL) was added triethylamine (60 μL, 0.411 mmol). The reaction was stirred at 65° C. for 15 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound as a yellow solid (5.4 mg). LC-MS m/z: (pos) 583.16 ([M+H]⁺).

### Example 21: Synthesis of N-(1-(3-((1-(2,6-dioxopiperidin-3-yl)-2,5-dioxo-2,5-dihydro-1H-pyrrol-3-yl)amino)phenyl)-2,2,2-trifluoroethyl)-2-(3-(trifluoromethyl)phenyl)acetamide (68)

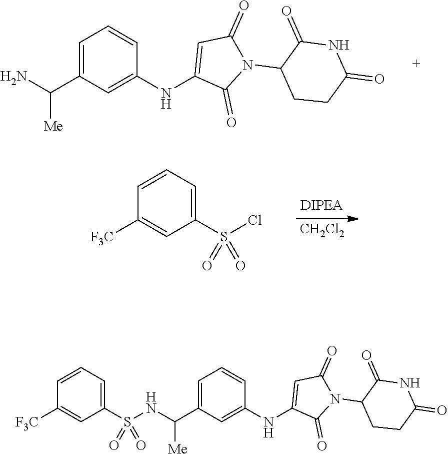

To a solution of 3-(3-((3-(1-aminoethyl)phenyl)amino)-2,5-dioxo-2,5-dihydro-1H-pyrrol-1-yl)piperidine-2,6-dione (42.1 mg, 0.123 mmol) and 3-(trifluoromethyl)benzenesulfonyl chloride (33 mg, 0.135 mmol) in CH₂Cl₂ (4 mL) was added DIPEA (52 μL, 0.298 mmol). The reaction was stirred at rt for 2 hours. Upon complete conversion of the amine, the reaction mixture was extracted with CH₂Cl₂, washed with H₂O, dried over Na₂SO₄, filtered, and concentrated in vacuo. Purification by prep TLC with 1:20 1.75 N NH₃ in MeOH/CH₂Cl₂ yielded the title compound. LC-MS m/z: (pos) 551.42 ([M+H]⁺), 573.42 ([M+Na]⁺).

### Example 22: Synthesis of N-(1-(3-((1-(2,6-dioxopiperidin-3-yl)-2,5-dioxo-2,5-dihydro-1H-pyrrol-3-yl)amino)phenyl)ethyl)-3,4-difluorobenzenesulfonamide (69)

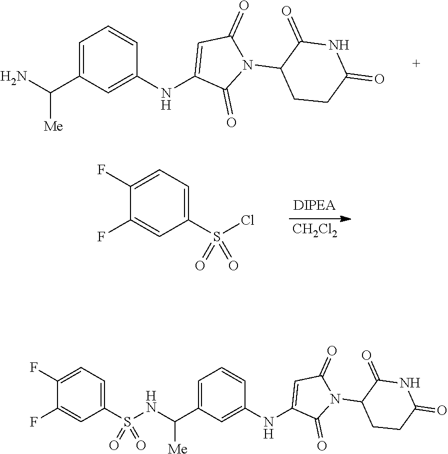

To a solution of 3-(3-((3-(1-aminoethyl)phenyl)amino)-2,5-dioxo-2,5-dihydro-1H-pyrrol-1-yl)piperidine-2,6-dione (49.3 mg, 0.144 mmol) and 3,4-difluorobenzenesulfonyl chloride (34 mg, 0.158 mmol) in CH₂Cl₂ (4 mL) was added DIPEA (50 μL, 0.288 mmol). The reaction was stirred at rt for 2 hours. Upon complete conversion of the amine, the reaction mixture was extracted with CH₂Cl₂, washed with H₂O, dried over Na₂SO₄, filtered, and concentrated in vacuo. Purification by prep TLC with 1:20 1.75 N NH₃ in MeOH/CH₂Cl₂ yielded the title compound. LC-MS m/z: (pos) 519.33 ([M+H]⁺), 541.39 ([M+Na]⁺).

### Example 23: Synthesis of 2-(4-chlorophenyl)-N-(1-(3-((1-(2,6-dioxopiperidin-3-yl)-2,5-dioxo-2,5-dihydro-1H-pyrrol-3-yl)amino)phenyl)ethyl)-2,2-difluoroacetamide (77)

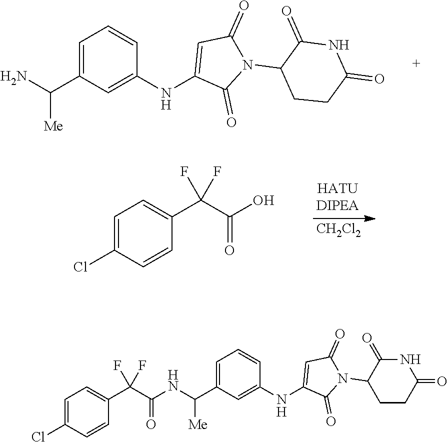

To a solution of 2-(4-chlorophenyl)-2,2-difluoroacetic acid (16.5 mg, 0.080 mmol) in CH₂Cl₂ (3 mL) was added HATU (33 mg, 0.088 mmol), DIPEA (40 μL, 0.22 mmol), then 3-(3-((3-(1-aminoethyl)phenyl)amino)-2,5-dioxo-2,5-dihydro-1H-pyrrol-1-yl)piperidine-2,6-dione (25 mg, 0.073 mmol), sequentially. The reaction mixture was stirred for 2 hours at rt. Upon complete conversion of the amine, the reaction mixture was extracted with CH₂Cl₂, washed with H₂O, dried over Na₂SO₄, filtered, and concentrated in vacuo. Purification by prep TLC with 1:20 1.75 N NH₃ in MeOH/CH₂Cl₂ yielded the title compound as a yellow solid (11.8 mg). LC-MS m/z: (pos) 531.09 ([M+H]⁺).

### Example 24: Synthesis of 3-((1-(2,6-dioxopiperidin-3-yl)-2,5-dioxo-2,5-dihydro-1H-pyrrol-3-yl)amino)-N-(3-(trifluoromethyl)phenethyl)benzenesulfonamide (82)

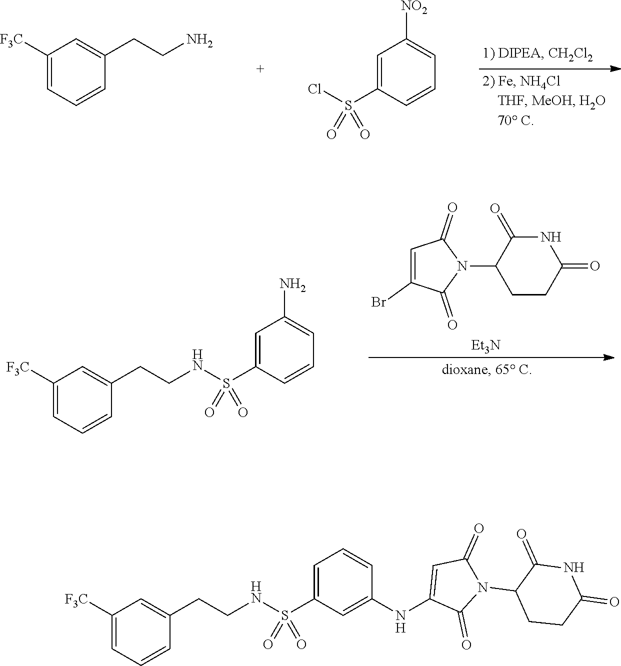

### 3-amino-N-(3-(trifluoromethyl)phenethyl)benzenesulfonamide

To a solution of 3-nitrobenzenesulfonyl chloride (201 mg, 0.91 mmol) in CH₂Cl₂ (4 mL) was added 2-(3-(trifluoromethyl)phenyl)ethan-1-amine (161 μL, 1.0 mmol) and DIPEA (317 μL, 1.82 mmol). The reaction mixture was stirred at rt for 2 hours. Upon complete conversion of the sulfonyl chloride, the reaction mixture was extracted with CH₂Cl₂, washed with H₂O, dried over Na₂SO₄, filtered, and concentrated in vacuo to give crude 3-nitro-N-(3-(trifluoromethyl)phenethyl)-benzenesulfonamide, which was taken forward without purification. LC-MS m/z: (pos) 374.31 ([M+H]⁺).

Crude 3-nitro-N-(3-(trifluoromethyl)phenethyl)-benzenesulfonamide (340 mg, 0.91 mmol) was dissolved in a solution of THF (4 mL) and MeOH (1 mL). Iron powder (152 mg, 2.7 mmol) was added, followed by 2 mL satd. aq. NH₄Cl. The reaction mixture was stirred at 70° C. for 3 hours. Upon cooling to rt, the reaction mixture was extracted with CH₂Cl₂, washed with H₂O, dried over Na₂SO₄, filtered over Celite®, and concentrated in vacuo to give the title compound (287.7 mg), which was taken forward without purification. LC-MS m/z: (pos) 345.23 ([M+H]⁺).

### 3-((1-(2,6-dioxopiperidin-3-yl)-2,5-dioxo-2,5-dihydro-1H-pyrrol-3-yl)amino)-N-(3-(tri-fluoromethyl)phenethyl)benzenesulfonamide

To a solution of 3-(3-bromo-2,5-dioxo-2,5-dihydro-1H-pyrrol-1-yl)piperidine-2,6-dione (96 mg, 0.335 mmol) and 3-amino-N-(3-(trifluoromethyl)phenethyl)benzenesulfonamide (150 mg, 0.436 mmol) in dioxane (4 mL) was added triethylamine (140 μL, 1.01 mmol). The reaction mixture was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound. LC-MS m/z: (pos) 551.43 ([M+H]⁺).

### Example 25: Synthesis of 2-((3-((1-(2,6-dioxopiperidin-3-yl)-2,5-dioxo-2,5-dihydro-1H-pyrrol-3-yl)amino)phenyl)-thio)-N-(3-(trifluoromethyl)phenyl)acetamide (83)

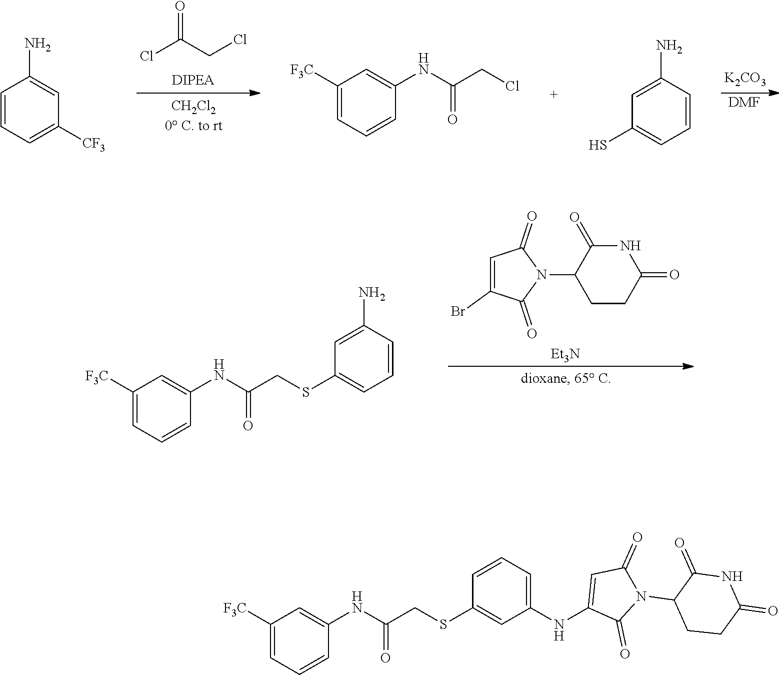

### 2-chloro-N-(3-(trifluoromethyl)phenyl)acetamide

Chloroacetyl chloride (107 μL, 1.34 mmol) was dissolved in CH₂Cl₂ (6 mL) and cooled to 0° C. 3-(Trifluoromethyl)aniline (154 mg, 0.956 mmol) was added dropwise at 0° C., followed by DIPEA (333 μL, 1.91 mmol). The reaction mixture was stirred at 0° C. for 10 minutes and then warmed to rt over 1 hour. Upon complete conversion of the aniline, the reaction mixture was extracted with CH₂Cl₂, washed with H₂O, dried over Na₂SO₄, filtered, and concentrated in vacuo to give the title compound, which was taken forward without purification.

### 2-((3-aminophenyl)thio)-N-(3-(trifluoromethyl)phenyl)acetamide

To a solution of 3-aminobenzenethiol (152 μL, 1.42 mmol) in DMF (6 mL) was added potassium carbonate (200 mg, 1.43 mmol) and stirred at rt for 15 minutes. 2-Chloro-N-(3-(trifluoromethyl)phenyl)acetamide was added to the reaction mixture and stirred at rt for 5 hours. Upon complete conversion of the acetamide, the reaction mixture was extracted with EtOAc, washed with H₂O ×2, brine, dried over Na₂SO₄, filtered, and concentrated in vacuo. Purification by silica flash chromatography yielded the title compound (270 mg). LC-MS m/z: (pos) 327.19 ([M+H]⁺).

### 2-((3-((1-(2,6-dioxopiperidin-3-yl)-2,5-dioxo-2,5-dihydro-1H-pyrrol-3-yl)amino)phenyl)-thio)-N-(3-(trifluoromethyl)phenyl)acetamide

To a solution of 3-(3-bromo-2,5-dioxo-2,5-dihydro-1H-pyrrol-1-yl)piperidine-2,6-dione (91.3 mg, 0.318 mmol) and 2-((3-aminophenyl)thio)-N-(3-(trifluoromethyl)phenyl)acetamide (135 mg, 0.414 mmol) in dioxane (6 mL) was added triethylamine (133 μL, 0.954 mmol). The reaction mixture was stirred at 65° C. for 16 hours. Upon cooling to rt, the reaction mixture was filtered over a short plug of silica and Celite®, and concentrated in vacuo. Purification by prep TLC with 1:20 1.75 N NH₃ in MeOH/CH₂Cl₂ yielded the title compound. LC-MS m/z: (pos) 533.44 ([M+H]⁺).

### Example 26: Synthesis of N-(3-chloro-4-methylphenyl)-2-[(3-{[1-(2,6-dioxopiperidin-3-yl)-2,5-dioxo-2,5-dihydro-1H-pyrrol-3-yl]amino}phenyl)(methyl)amino]acetamide (44)

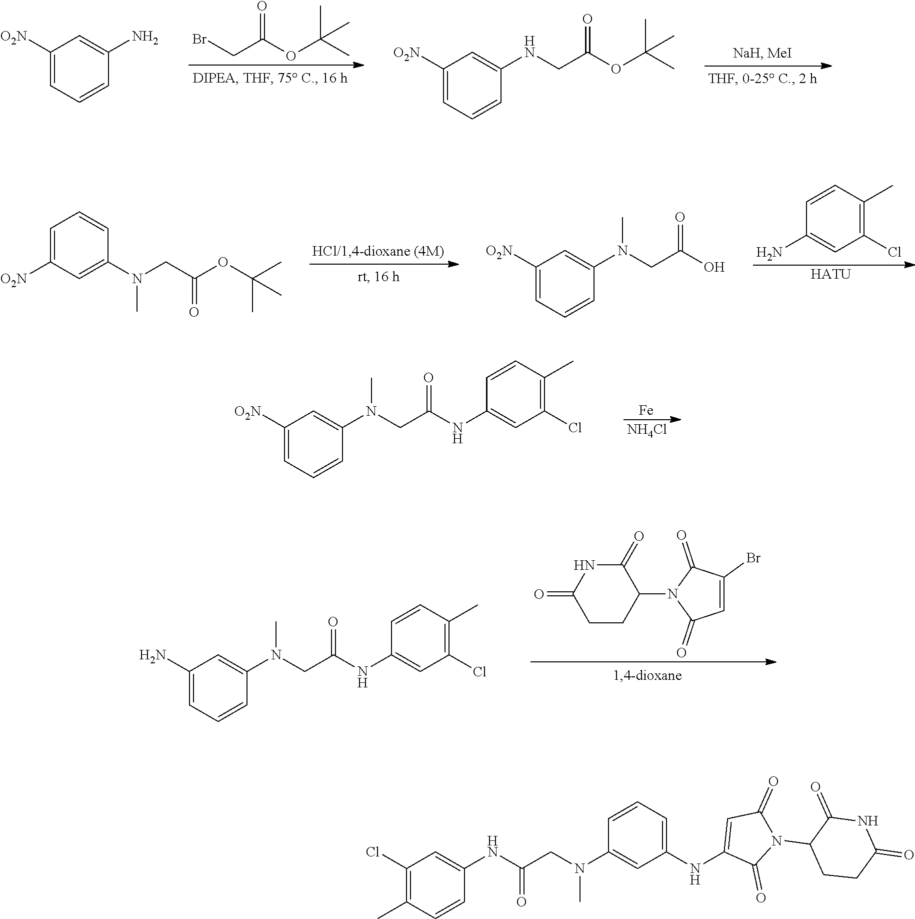

### (3-Nitro-phenylamino)-acetic acid tert-butyl ester

A solution of 3-nitro-phenylamine (1.0 g, 7.24 mmol) and bromo-acetic acid tert-butyl ester (1.41 g, 7.24 mmol) in 50 mL of THF was treated with N,N-diisopropylethylamine (2.81 g, 21.72 mmol). The reaction mixture was stirred overnight at 75° C. The reaction was diluted with water and extracted with ethyl acetate (50 mL×3). The layers were separated and the combined organic layers were concentrated under reduced pressure. The residue was purified by column chromatography (eluted with DCM/MeOH=20/1) to afford the title compound as a white solid (134 mg, 7.34%). LC-MS m/z: (pos) 253.2 ([M+H]⁺).

### [Methyl-(3-nitro-phenyl)-amino]-acetic acid tert-butyl ester

NaH (12.8 mg, 0.53 mmol) was added to a solution of (3-nitro-phenylamino)-acetic acid tert-butyl ester (134 mg, 0.53 mmol) in THF (10 mL) and the reaction mixture was stirred for 1.5 hours at 0° C. Mel (0.3 mL, 1.06 mmol) was then added and the reaction mixture was stirred at rt for 2 hours. The reaction mixture was diluted with H₂O (50 mL) and extracted with ethyl acetate (50 mL×3). The layers were separated and the combined organic layers was concentrated under reduced pressure. The residue was purified by column chromatography (eluted with DCM/MeOH=20/1) to afford the title compound as a white solid (128 mg, 99.0%). LC-MS m/z: (pos) 267.18 ([M+H]⁺).

### [Methyl-(3-nitro-phenyl)-amino]-acetic acid

HCl-dioxane (4 M, 10 mL) was added to [methyl-(3-nitro-phenyl)-amino]-acetic acid tert-butyl ester (128 mg, 0.48 mmol) in a sealed vial and the suspension was stirred at rt for 16 hours. The solvent was removed under reduced pressure to afford the title compound as a white solid (125 mg, 100%) which was used without further purification. LC-MS m/z: (pos) 211.10 ([M+H]⁺).

### N-(3-Chloro-4-methyl-phenyl)-2-[methyl-(3-nitro-phenyl)-amino]-acetamide

N,N-diisopropylethylamine (229.2 mg, 1.77 mmol) and HATU (448.7 mg, 1.18 mmol) were added to a solution of [methyl-(3-nitro-phenyl)-amino]-acetic acid (125 mg, 0.59 mmol) and 3-chloro-4-methyl-phenylamine (83.2 mg, 0.59 mmol) in DMF (10 mL) and the reaction mixture stirred at rt for 16 hours. The reaction mixture was concentrated under reduced pressure to afford a residue, that was then suspended in water (50 mL). The solid was filtered and washed with water (3×20 mL) and dried to afford the title compound as a white solid (130 mg, 65.6%) that was used without further purification. LC-MS m/z: (pos) 334.10 ([M+H]⁺).

### 2-[(3-Amino-phenyl)-methyl-amino]-N-(3-chloro-4-methyl-phenyl)-acetamide

NH₄Cl (31.3 mg, 0.59 mmol, dissolved in 1 mL of H₂O) and Fe powder (43.7 mg, 0.78 mmol) were added to a solution of N-(3-chloro-4-methyl-phenyl)-2-[methyl-(3-nitro-phenyl)-amino]-acetamide (130 mg, 0.39 mmol) in THF (3 mL) and the reaction mixture was stirred at 70° C. for 4 hours. The reaction mixture was filtered, and the filtrate was concentrated under reduced pressure to afford a residue that was then dissolved in EtOAc (30 mL) and water (20 mL). The layers were separated. The organic phase was concentrated to afford the title compound as a brown oil (115 mg, 99.0%) that was used without further purification. LC-MS m/z: (pos) 304.10 ([M+H]⁺).

### N-(3-Chloro-4-methyl-phenyl)-2-({3-[1-(2,6-dioxo-piperidin-3-yl)-2,5-dioxo-2,5-dihydro-1H-pyrrol-3-ylamino]-phenyl}-methyl-amino)-acetamide (44)

The title compound was isolated as a yellow solid (32 mg, 16.5%) following a similar procedure as in Example 15. LC-MS m/z: (pos) 510.20 ([M+H]⁺).

### Example 27: Liquid Chromatography Mass Spectrometry (LCMS) Data

Reaction monitoring and final compound characterization were performed using Shimadzu LC-20AD series (binary pump and diode array detector) with Agilent Poroshell 120 EC-C18 column (2.7 μm, 4.6×50 mm). Mobile phase: A: 0.05% Formate in water (v/v), B: 0.05% Formate in MeCN (v/v). Flow Rate: 1 mL/min at 25° C. MS: 2020, Quadrupole LC/MS, Ion Source: API-ESI, TIC: 100-900 m/z; Drying gas flow: 15 L/min; Nebulizer pressure: 1.5 L/min; Drying gas temperature: 250° C., Vcap: 4500V.

TABLE 1
LCMS Data

| | Observed Mass |
|---|---|
| Compound # | (M + H)⁺ |
| 7 | 496.24 |
| 8 | 496.24 |
| 9 | 496.23 |
| 16 | 524.26 |
| 18 | 565.33 |
| 19 | 537.23 |
| 20 | 538.27 |
| 21 | 507.23 |
| 22 | 541.25 |
| 23 | 556.23 |
| 25 | 459.31 |
| 26 | 473.32 |
| 27 | 475.28 |

### Example 28: Potency of Inventive Compounds

### HiBit-IKZF2 and HiBit-IKZF1 Assays

The HiBit tag was knocked into the endogenous IKZF2 or IKZF1 locus of Molt4 cells to generate a HiBit-IKZF2 or HiBit-IKZF1 fusion proteins via CRISPR/Cas9 gene editing. Cells were then treated with a 12 point concentration curve of the indicated compounds, and luminescence (corresponding to Helios abundance) was detected after 6 hours using the Nano-Glo® HiBit Lytic Detection system (Promega™). The concentration necessary to achieve 50% degradation (DC₅₀) was calculated using Prism (GraphPad). The results are shown in Table 2.

TABLE 2
Degradation Activity (HiBit-IKZF2 and HiBit-IKZF1 assays)

| Compound Number | IKZF2 DC₅₀ (nM) | IKZF1 DC₅₀ (nM) |
|---|---|---|
| 36 | 2.5 | 15 |
| 60 | 42 | 240 |
| 65 | 17 | 18 |
| 66 | 2.3 | 3.8 |
| 67 | 25 | 47 |

### Degradation in Jurkat Cells

Cells were lysed in M-PER® buffer (Thermo Scientific) containing protease/phosphatase inhibitor cocktail (Roche). Protein concentration was measured using a BCA assay (Pierce™). Equivalent amounts of each sample were loaded on 4-12% Bis-Tris gels (Invitrogen™), transferred to nitrocellulose membranes, and immunoblotted with the indicated antibodies. IRDye®800-labeled goat anti-rabbit IgG and IRDye®680-labeled goat anti-mouse IgG (LI-COR®) secondary antibodies were purchased from LI-COR®, and membranes were detected on an Odyssey® detection system (LI-COR® Biosciences).

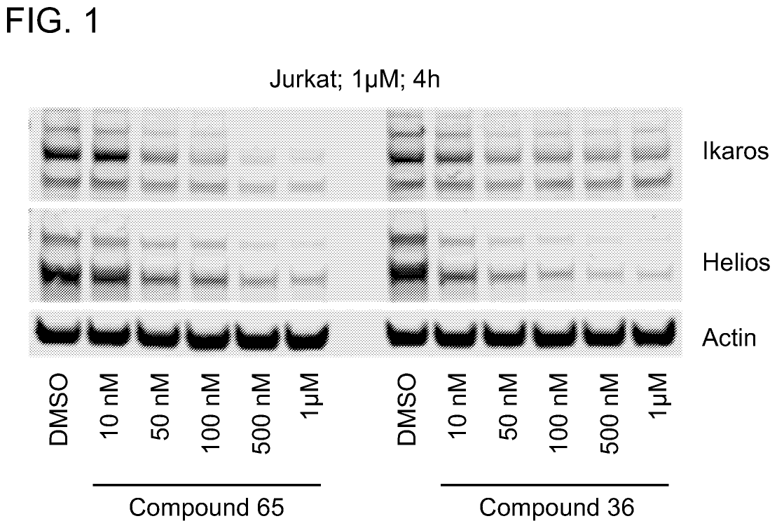

Immunoblots for Ikaros, Helios, and Actin from wildtype Jurkat cells treated with compound 36 or 65 for 4 hours at the indicated concentrations were generated (FIG. 1).

### Example 29: Cellular Degradation Assays

IKZF1A and IKZF2A were subcloned into mammalian pcDNA5/FRT Vector (Ampicillin and Hygromycin B resistant) modified to contain MCS-eGFP-P2A-mCherry. Stable cell lines expressing eGFP-protein fusion and mCherry reporter were generated using Flip-In 293 system. Plasmid (0.3 μg) and pOG44 (4.7 μg) DNA were preincubated in 100 μL of Opti-MEM™ I (Gibco™, Life Technologies™) media containing 0.05 mg/mL Lipofectamine™ 2000 (Invitrogen™) for 20 min and added to Flip-In 293 cells containing 1.9 mL of DMEM media (Gibco™, Life Technologies™) per well in a 6-well plate format (Falcon, 353046). Cells were propagated for 48 h and were then transferred into a 10 cm² plate (Corning, 430165) in DMEM media containing 50 μg/mL of Hygromycin B (REF 10687010, Invitrogen™) as a selection marker. Following 2-3 passage cycle, FACS (FACSAria™ II, BD) was used to enrich for cells expressing eGFP and mCherry.

Cells stably expressing the IKZF1A, or IKZF2A GFP fusions with mCherry reporter were seeded at 30-50% confluency in 384-well plates with 50 μL FluoroBrite™ DMEM media (Thermo Fisher Scientific™ A18967) containing 10% FBS per well a day before compound treatment. Compound titrations were dispensed using a D300e Digital Dispenser (HP), normalized to 0.5% DMSO, and incubated with cells for 5 hours.

The assay plate was imaged immediately using an Acumen High Content Imager (TTP Labtech) with 488 nm and 561 nm lasers in 2 μm×1 μm grid per well format. The resulting images were analyzed using CellProfiler (Carpenter et al., Genome Biol. 2006, 7:R100). A series of image analysis steps (‘image analysis pipeline’) was constructed. First, the red and green channels were aligned and cropped to target the middle of each well (to avoid analysis of heavily clumped cells at the edges), and a background illumination function was calculated for both red and green channels of each well individually and subtracted to correct for illumination variations across the 384-well plate from various sources of error. An additional step was then applied to the green channel to suppress the analysis of large auto fluorescent artifacts and enhance the analysis of cell specific fluorescence by way of selecting for objects under a given size, 30 A.U., and with a given shape, speckles. mCherry-positive cells were then identified in the red channel filtering for objects between 8-60 pixels in diameter and using intensity to distinguish between clumped objects. The green channel was then segmented into GFP positive and negative areas and objects were labeled as GFP positive if at least 40% of it overlapped with a GFP positive area. The fraction of GFP-positive cells/mCherry-positive cells in each well was then calculated, and the green and red images were rescaled for visualization. The concentrations that lead to half degradation at 5 h (DC_{50, 5h}) were calculated using the nonlinear fit variable slope model (GraphPad Software). The results are shown in Table 3.

TABLE 3
Degradation Activity (GFP-IKZF2 and GFP-IKZF1 assays)

| Compound | GFP IKZF2 | GFP IKZF1 |
|---|---|---|
| Number | DC₅₀ (nM) | DC₅₀ (nM) |
| 7 | 48 | 23 |
| 8 | 17 | 10 |
| 9 | 85 | 80 |
| 16 | 24 | 4.4 |
| 18 | 8.8 | 39 |
| 20 | 1.7 | 0.9 |
| 21 | 4.4 | 2.0 |
| 22 | 134 | 14 |
| 23 | 101 | 38 |
| 25 | 13 | 5.8 |
| 26 | 8.9 | 2.8 |
| 27 | 19 | 6.4 |
| 30 | 2.2 | 50 |
| 36 | 2.3 | 5.4 |
| 44 | 6.9 | 3.7 |
| 48 | 19 | 3.7 |
| 60 | 6.0 | 18 |
| 64 | 9.2 | 6.8 |
| 65 | 3.6 | 5.1 |
| 66 | 1.2 | 1.1 |
| 67 | 12 | 11 |
| 73 | 4.4 | 5.2 |
| 80 | 24 | 50 |

The data demonstrate that the compounds of the invention degrade IKZF2 or IKZF1 in cells.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A compound represented by a structure of formula I: 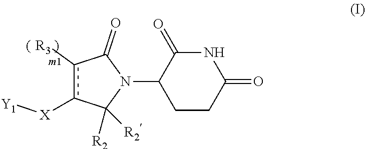
or a pharmaceutically acceptable salt, hydrate, solvate, prodrug, stereoisomer, or tautomer thereof,
wherein:
R₂, R₂′ are independently hydrogen, (C₁-C₆)alkyl, or (C₁-C₆)haloalkyl, or
R₂ and R₂′ together form ═O;
each R₃ is independently hydrogen, halogen, (C₁-C₆)alkyl, or (C₁-C₆)haloalkyl;
X is —O—, —S—, —NR₆—, or —C(R₅)₂—;
Y₁ is 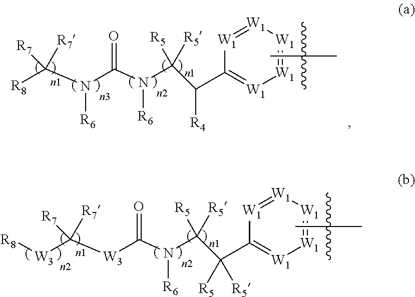
provided that when R₂ and R₂′ together form ═O, R₃ is H, X is NH, W₁ is CH, n₁ is 0, n₂ is 0, W₃ is NH, and R₈ is optionally substituted aryl, one of R₅ and R₅′ is not H, 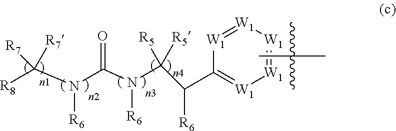
provided that when R₂ and R₂′ together
form ═O, R₃ is H, X is NH, W₁ is CH, R₆ is H, n₄ is 1, n₂ is 0, n₃ is 1, R₆ is H, n₁ is 0, and R₈ is optionally substituted aryl, one of R₅ and R₅′ is not H, 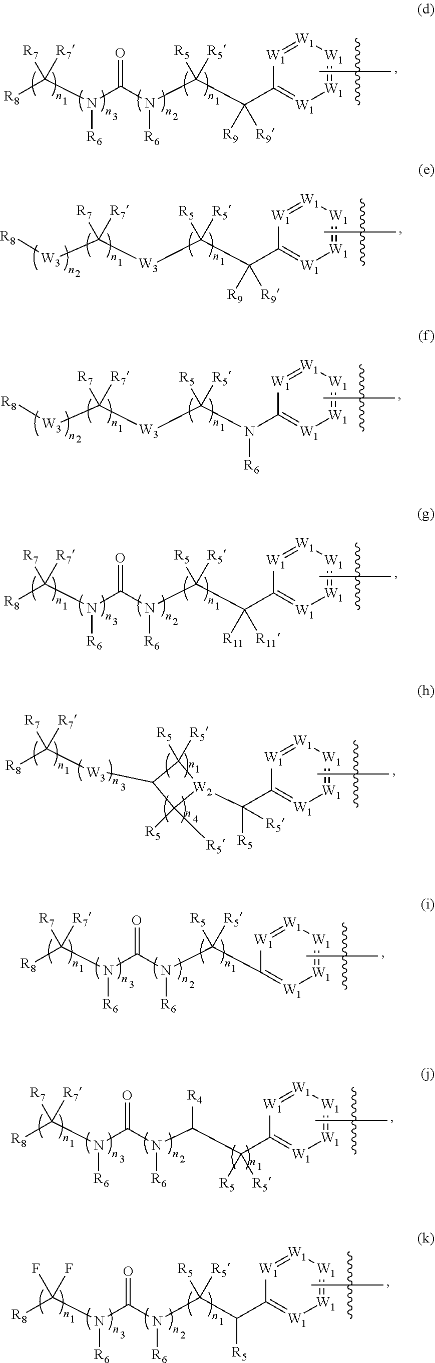 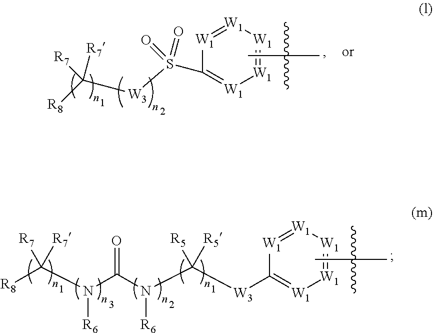
R₄ is —CF₃, 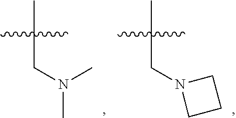
(C₃-C₇)alkyl, (C₃-C₇)haloalkyl, (C₃-C₇)cycloalkyl, 4- to 7-membered heterocycloalkyl, (C₆-C₁₀)aryl, and monocyclic or bicyclic 5- to 10-membered heteroaryl, —N(R₆)₂, —OR₆, 4- to 7-membered heterocycloalkyl which contains at least one nitrogen atom and is linked via the nitrogen atom, (C₂-C₆)alkenyl, or (C₂-C₆)alkynyl; wherein said alkyl, cycloalkyl, heterocycloalkyl, aryl, or heteroaryl is further optionally and independently substituted by one or more identical or different R₁₀ groups;
R₅ and R₅′ are absent or independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups, or
R₅ and R₅′ together with the same carbon atom to which they are attached form a spiro (C₃-C₇)cycloalkyl group or a 4- to 7-membered heterocycloalkyl group, or
R₅ and R₅′, when on different carbon atoms, together with the atoms to which they are attached form a (C₃-C₇)cycloalkyl group or a 4- to 7-membered heterocycloalkyl group, or
R₅ and R₅′, when on adjacent atoms, together with the atoms to which they are attached form a (C₆-C₁₀)aryl or a 5- or 6-membered heteroaryl; wherein said cycloalkyl, heterocycloalkyl, aryl or heteroaryl is further optionally and independently substituted by one or more identical or different R₁₀ groups, or
R₅ and R₉, together with the atoms to which they are attached form a (C₃-C₇)cycloalkyl group or a 4- to 7-membered heterocycloalkyl group; wherein said cycloalkyl, or heterocycloalkyl is further optionally and independently substituted by one or more identical or different R₁₀ groups;
R₆ is hydrogen, (C₁-C₆)alkyl, (C₁-C₆)haloalkyl, (C₃-C₇)cycloalkyl, 4- to 7-membered heterocycloalkyl, (C₆-C₁₀)aryl, or monocyclic or bicyclic 5- to 10-membered heteroaryl;
wherein said alkyl, cycloalkyl, heterocycloalkyl, aryl or heteroaryl is further optionally and independently substituted by one or more identical or different R₁₀ groups, or
two R₆ when on different nitrogen atoms, together with the atoms to which they are attached form a 4- to 7-membered heterocycloalkyl group; wherein said heterocycloalkyl is further optionally and independently substituted by one or more identical or different R₁₀ groups, or
R₆ and R₇, together with the atoms to which they are attached form a 4- to 8-membered heterocycloalkyl group; wherein said heterocycloalkyl is further optionally and independently substituted by one or more identical or different R₁₀ groups;
R₇ and R₇′ are absent or independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups, or
R₇ and R₇′ together with the same carbon atom to which they are attached form a spiro (C₃-C₇)cycloalkyl group or a 4- to 7-membered heterocycloalkyl group, or
R₇ and R₇′, when on different carbon atoms, together with the atoms to which they are attached form a (C₃-C₇)cycloalkyl group or a 4- to 7-membered heterocycloalkyl group, or
R₇ and R₇′, when on adjacent atoms, together with the atoms to which they are attached form a (C₆-C₁₀)aryl or a 5- or 6-membered heteroaryl; wherein said cycloalkyl, heterocycloalkyl, aryl or heteroaryl is further optionally and independently substituted by one or more identical or different R₁₀ groups;
R₈ is a (C₆-C₁₀)aryl, monocyclic or bicyclic 5- to 10-membered heteroaryl, wherein said aryl or heteroaryl is further optionally and independently substituted by one or more identical or different groups selected from R₁₀ or 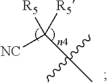
R₉ and R₉′ are independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups, or
R₉ and R₅, together with the atoms to which they are attached form a (C₃-C₇)cycloalkyl group or a 4- to 7-membered heterocycloalkyl group; wherein said cycloalkyl, or heterocycloalkyl is further optionally and independently substituted by one or more identical or different R₁₀ groups, or
R₉ and R₉′ together with the same carbon atom to which they are attached form a spiro (C₃-C₇)cycloalkyl group or a 4- to 7-membered heterocycloalkyl group;
each R₁₀ is independently alkyl, alkenyl, alkynyl, halo, haloalkyl, cycloalkyl, heterocycloalkyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, phosphinyl, phosphoryl including phosphine oxide and phosphonate, cyclic acetal, 4- to 7-membered heterocycloalkyl which contains at least one nitrogen atom and is linked via the nitrogen atom, aryl, or heteroaryl,
or wherein two adjacent R₁₀ groups taken together with the respective atoms to which each is attached form an aryl, or a heteroaryl, or a 5- to 8-membered cycloalkyl or 5- to 8-membered heterocycloalkyl;
R₁₁ and R₁₁′ are independently (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups, provided that R₁₁ and R₁₁′ are both not methyl;
R₂₁ is a monocyclic or bicyclic 5- to 10-membered heteroaryl; wherein said heteroaryl is further optionally and independently substituted by one or more identical or different R₁₀ groups;
m₁ is 0, 1, or 2;
n₁ is independently 0, 1, 2, or 3;
n₂ and n₃ are independently 0 or 1, provided that n₂ and n₃ cannot both be 0;
n₄ is 1, 2, 3, 4, or 5;
n₅ is 2, 3, 4, or 5;
W₁ is N, CR₅, or a carbon atom that is the point of attachment;
W₂ is N or CR₅; and
W₃ is —O—, —S—, —NR₆—, or —S(O₂)—.

2. The compound of claim 1, which is: 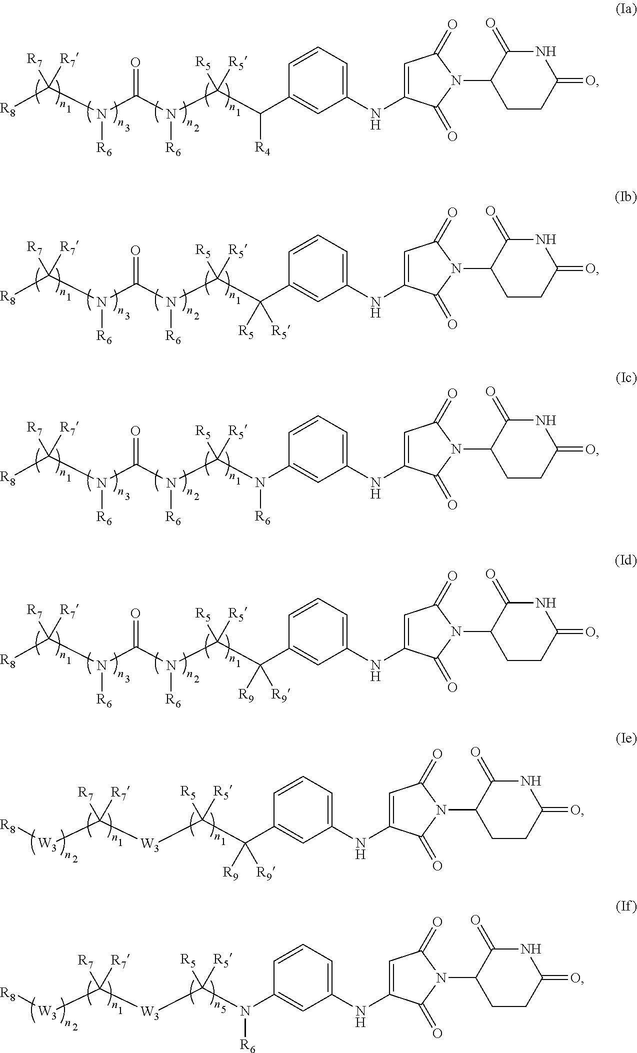 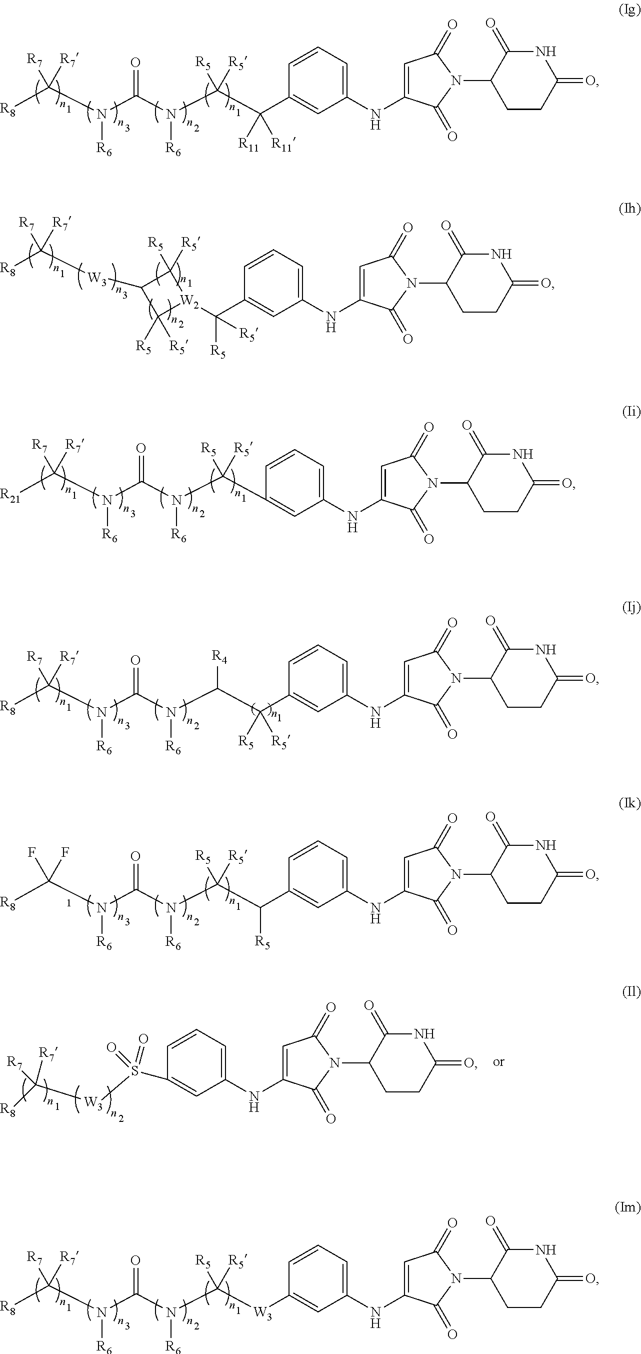
or a pharmaceutically acceptable salt, hydrate, solvate, prodrug, stereoisomer, or tautomer thereof.

3. The compound of claim 1, wherein Y₁ is

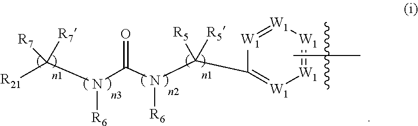

4. The compound of claim 1, R₅ and R₅′ are absent or independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups.

5. The compound of claim 4, wherein R₅ and R₅′ are independently hydrogen, methyl, ethyl, isopropyl, phenyl, chloro, fluoro, —NH₂, —NMe₂, —CF₃

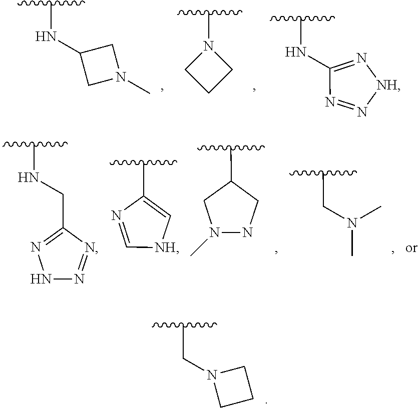

6. The compound of claim 1, wherein R₅ and R₅′ together with the same carbon atom to which they are attached form a spiro (C₃-C₇)cycloalkyl group or a 4- to 7-membered heterocycloalkyl group.

7. The compound of claim 6, wherein R₅ and R₅′ together with the same carbon atom to which they are attached form a spiro-cycle which is a cyclopropyl, cyclobutyl, oxetane, azetidine, or tetrahydropyran spiro-cycle.

8. The compound of claim 1, wherein R₅ and R₅′, when on different carbon atoms, together with the atoms to which they are attached form a (C₃-C₇)cycloalkyl group or a 4- to 7-membered heterocycloalkyl group; wherein said cycloalkyl or heterocycloalkyl is further optionally and independently substituted by one or more identical or different R₁₀ groups.

9. The compound of claim 8, wherein R₅ and R₅′ when on different carbon atoms, together with the atoms to which they are attached form a cyclopropyl or cyclobutyl ring.

10. The compound of claim 1, wherein R₇ and R₇′ are absent or independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups.

11. The compound of claim 10, wherein R₇ and R₇′ are independently hydrogen, methyl, ethyl, isopropyl, phenyl, chloro, fluoro, —NH₂, —NMe₂, —CF₃,

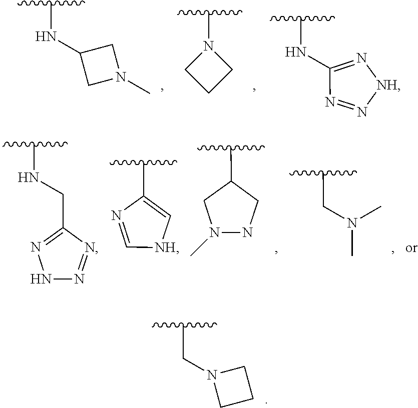

12. The compound of claim 1, wherein R₇ and R₇′ together with the same carbon atom to which they are attached, form a spiro (C₃-C₇)cycloalkyl group or a 4- to 7-membered heterocycloalkyl group.

13. The compound of claim 12, wherein R₇ and R₇′ together with the same carbon atom to which they are attached, form a spiro-cycle which is a cyclopropyl, cyclobutyl, oxetane, azetidine, or tetrahydropyran spiro-cycle.

14. The compound of claim 1, wherein R₇ and R₇′, when on different carbon atoms, together with the atoms to which they are attached, form a (C₃-C₇)cycloalkyl group or a 4- to 7-membered heterocycloalkyl group; wherein said cycloalkyl or heterocycloalkyl is further optionally and independently substituted by one or more identical or different R₁₀ groups.

15. The compound of claim 14, wherein R₇ and R₇′, when on different carbon atoms, together with the atoms to which they are attached, form a cyclopropyl or cyclobutyl ring.

16. The compound of claim 1, wherein R₈ is

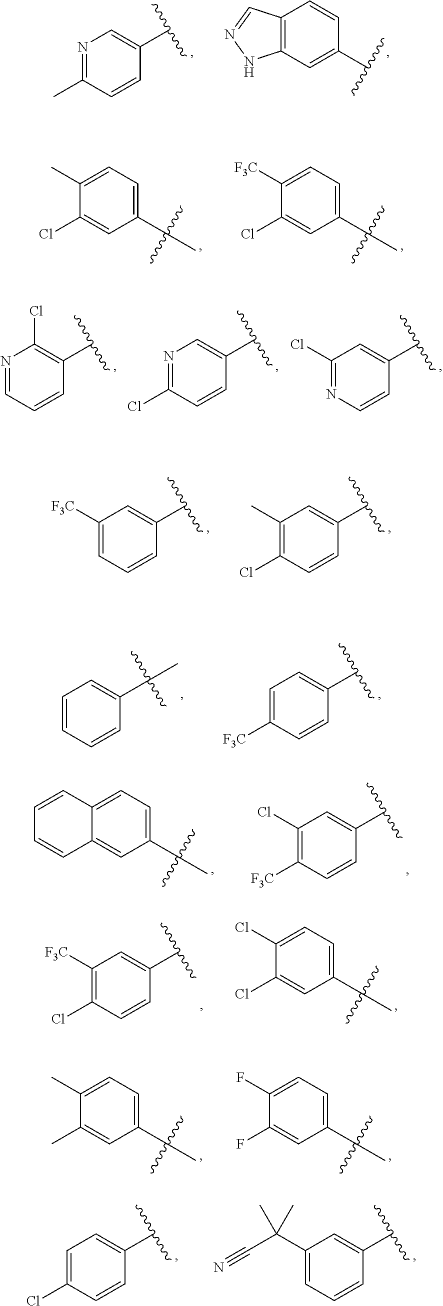

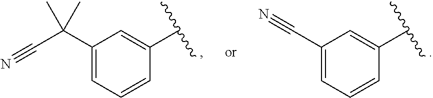 [SMILES: *c1cccc(C(C)(C)C#N)c1 ; *c1cccc(C#N)c1]

17. The compound of claim 1, wherein R₉ and R₉′ are independently hydrogen, (C₁-C₆)alkyl, (C₁-C₆)haloalkyl, (C₁-C₆)hydroxyalkyl, (C₃-C₇)cycloalkyl, 4- to 7-membered heterocycloalkyl, (C₆-C₁₀)aryl, monocyclic or bicyclic 5- to 10-membered heteroaryl, halogen, cyano, —N(R₆)₂, —OR₆, 4- to 7-membered heterocycloalkyl which contains at least one nitrogen atom and is linked via the nitrogen atom, amido, carbonyl, (C₂-C₆)alkenyl, or (C₂-C₆)alkynyl; wherein said alkyl, alkynyl, cycloalkyl, heterocycloalkyl, aryl, or heteroaryl is further optionally and independently substituted by one or more identical or different R₁₀ groups.

18. The compound of claim 17, wherein R₉ and R₉′ are independently hydrogen, methyl, ethyl, isopropyl, phenyl, chloro, fluoro, —NH₂, —NMe₂, —CF₃,

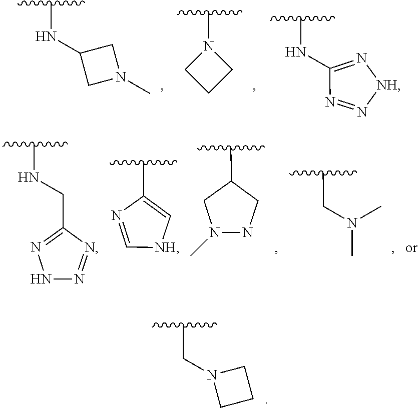

19. The compound of claim 1, wherein R₉ and R₉′ together with the same carbon atom to which they are attached, form a spiro (C₃-C₇)cycloalkyl group or a 4- to 7-membered heterocycloalkyl group.

20. The compound of claim 19, wherein R₉ and R₉′ together with the same carbon atom to which they are attached, form a spiro-cycle which is a cyclopropyl, cyclobutyl, oxetane, azetidine, or tetrahydropyran spiro-cycle.

21. The compound of claim 1, wherein R₂₁ is

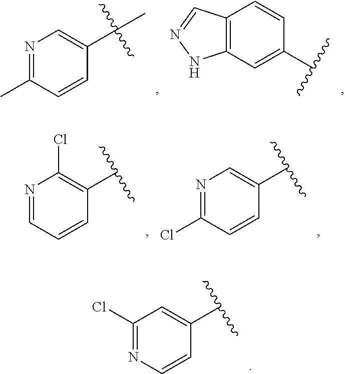

22. The compound of claim 1, wherein m₁ is 0 or 1.

23. The compound of claim 1, wherein n₁ is 0 or 1.

24. The compound of claim 1, wherein n₂ is 0 or 1.

25. The compound of claim 1, wherein n₃ is 0 or 1.

26. The compound of claim 1, wherein n₄ is 2.

27. The compound of claim 1, wherein n₅ is 2.

28. The compound of claim 1, wherein W₁ is N.

29. The compound of claim 1, W₁ is CR₅.

30. The compound of claim 1, wherein W₂ is N.

31. The compound of claim 1, W₂ is CR₅.

32. The compound of claim 1, wherein W₃ is —NR₆—.

33. The compound of claim 1, wherein W₃ is —S(O)₂—.

34. The compound of claim 1, which is: 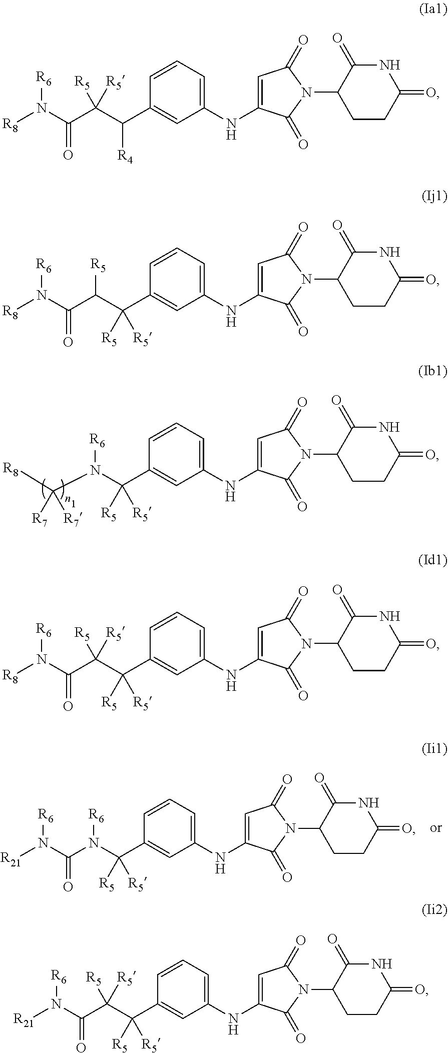
or a pharmaceutically acceptable salt, hydrate, solvate, prodrug, stereoisomer, or tautomer thereof,
wherein
R₆ is hydrogen or (C₁-C₆)alkyl;
n₁ is 1 or 2;
R₉ and R₅, together with the atoms to which they are attached form a (C₃-C₇)cycloalkyl group or a 4- to 7-membered heterocycloalkyl group: wherein said cycloalkyl, or heterocycloalkyl is further optionally and independently substituted by one or more identical or different R₁₀ groups; and
R₂₁ is a monocyclic 6-membered heteroaryl; wherein said heteroaryl is further optionally and independently substituted by one or more identical or different R₁₀ groups.

35.-37. (canceled)

38. The compound of claim 1, which is: 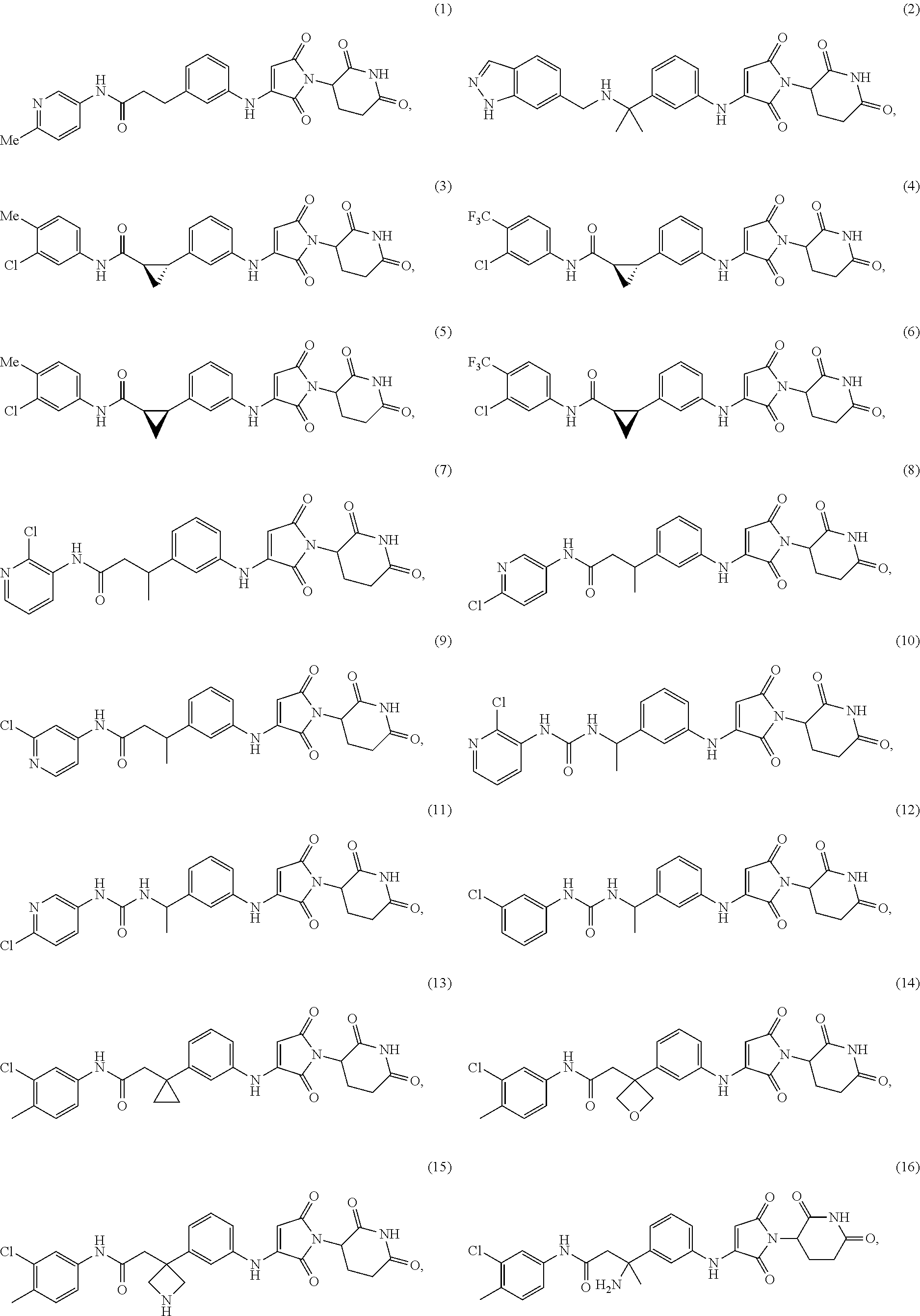 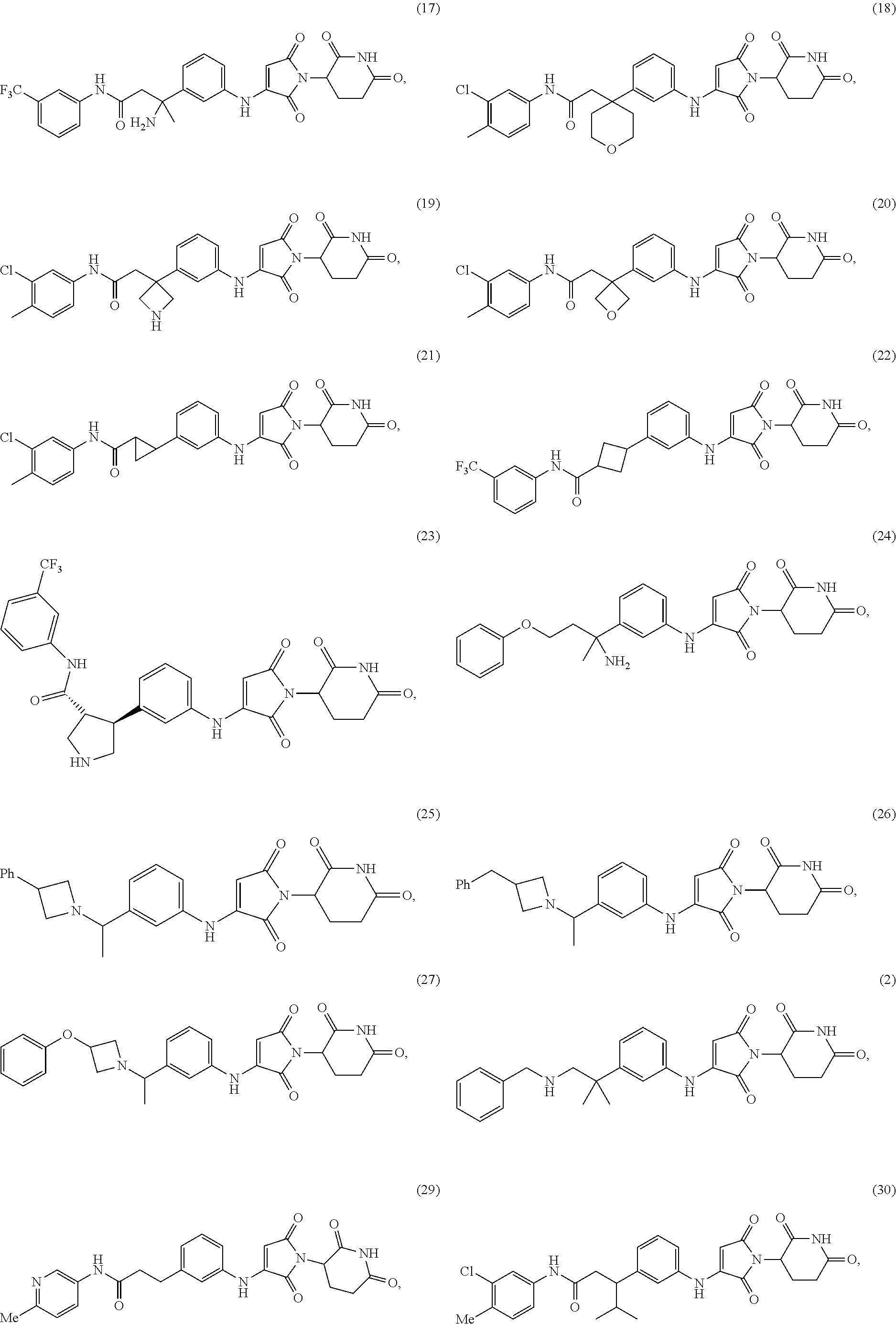 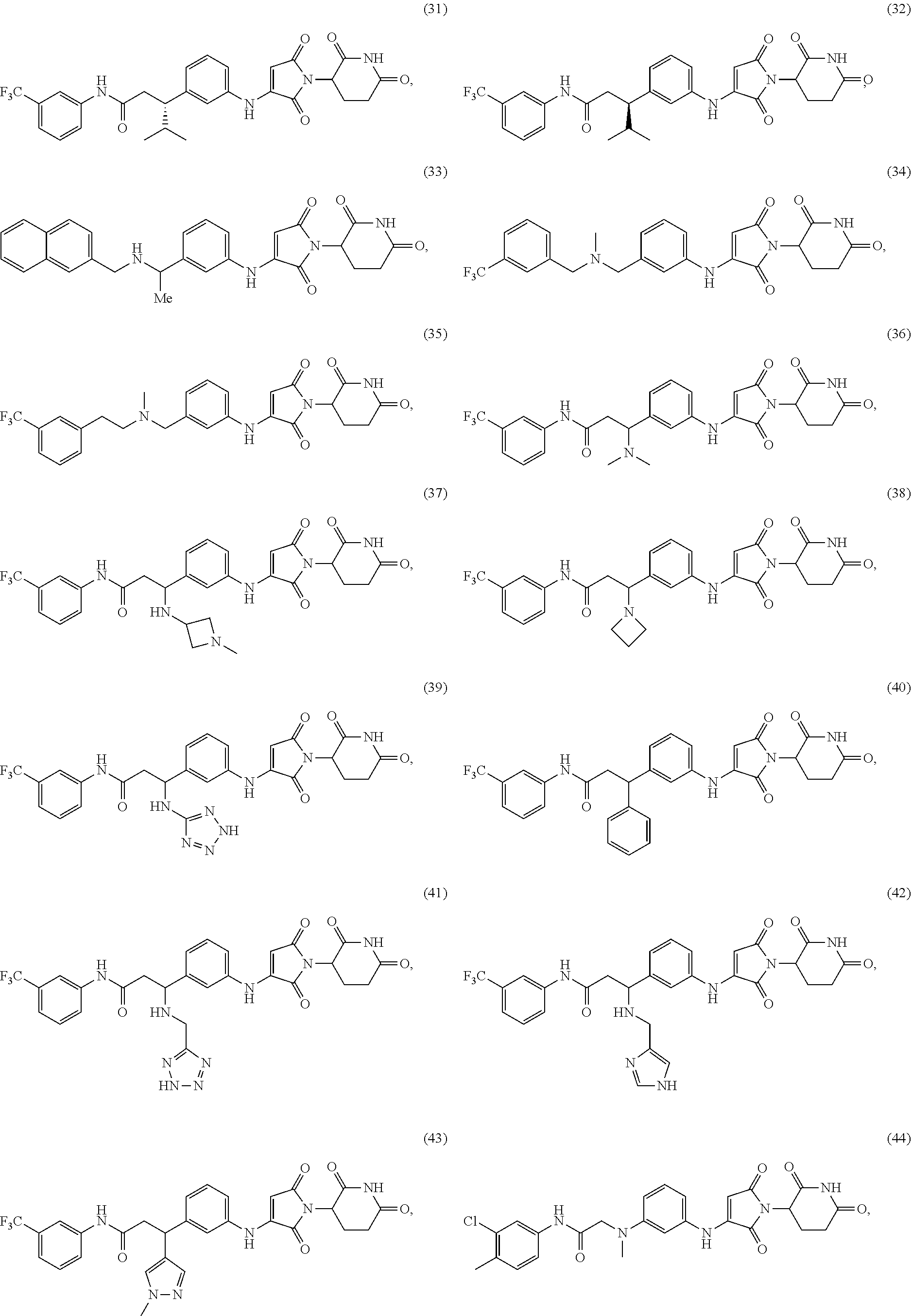 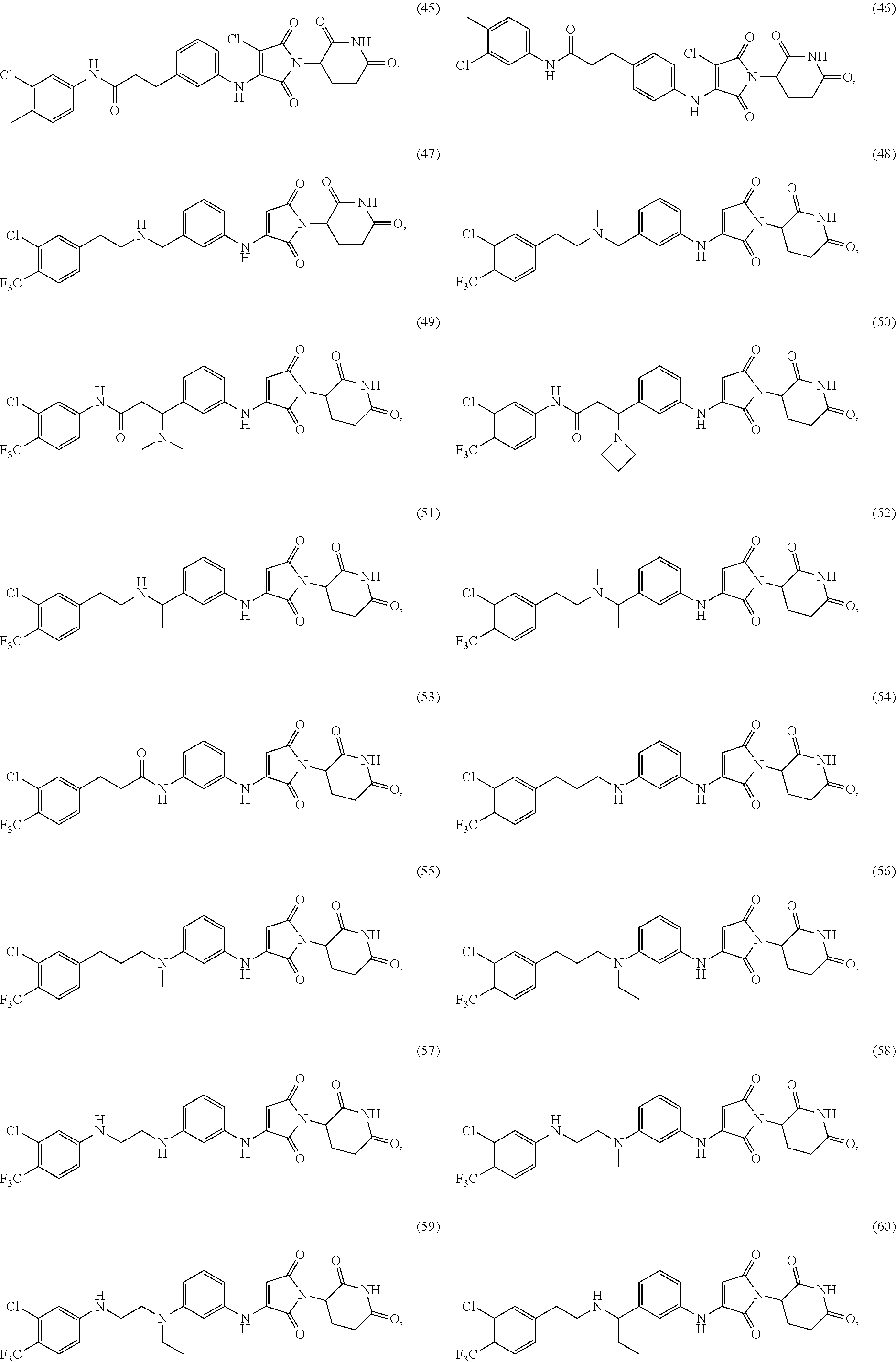 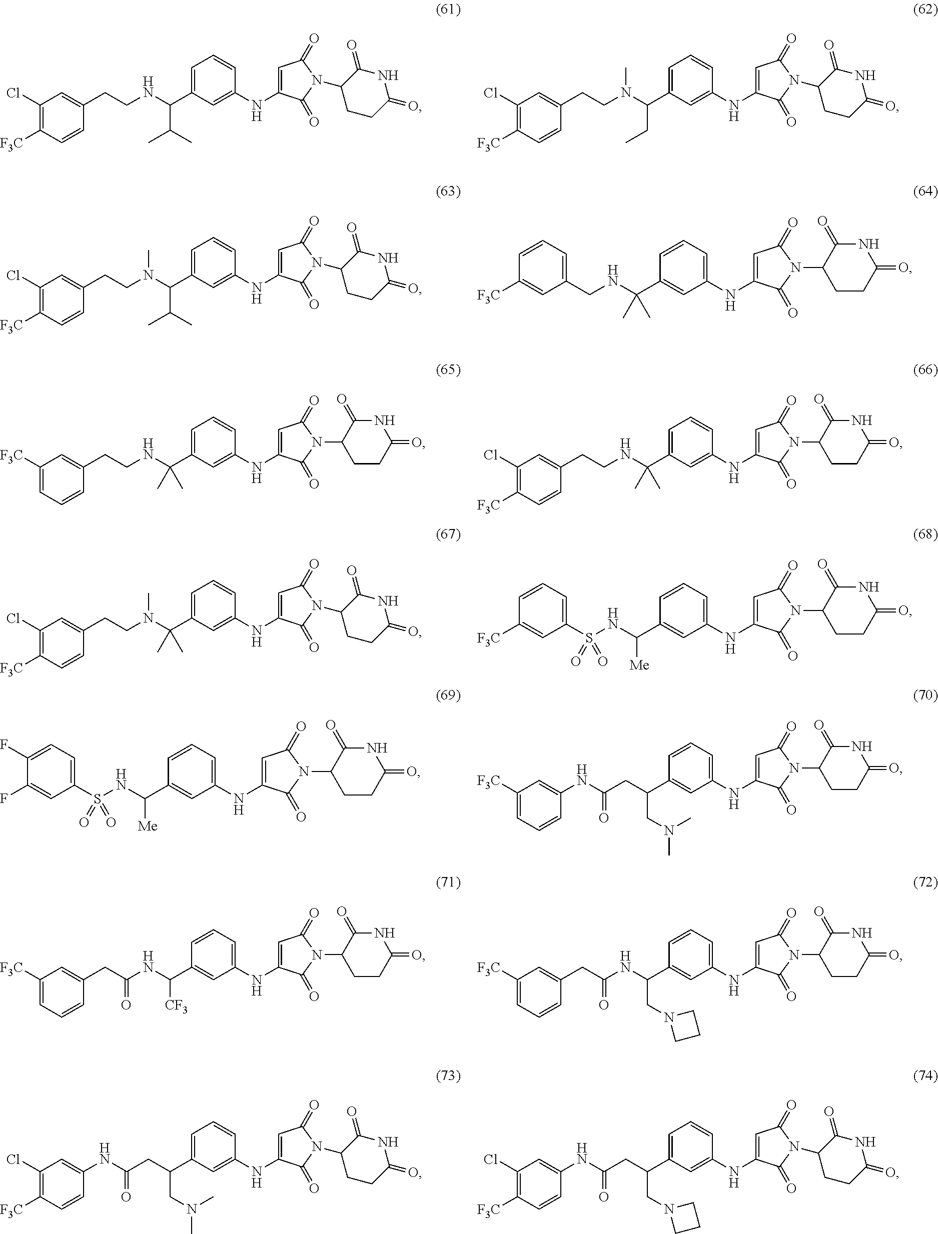 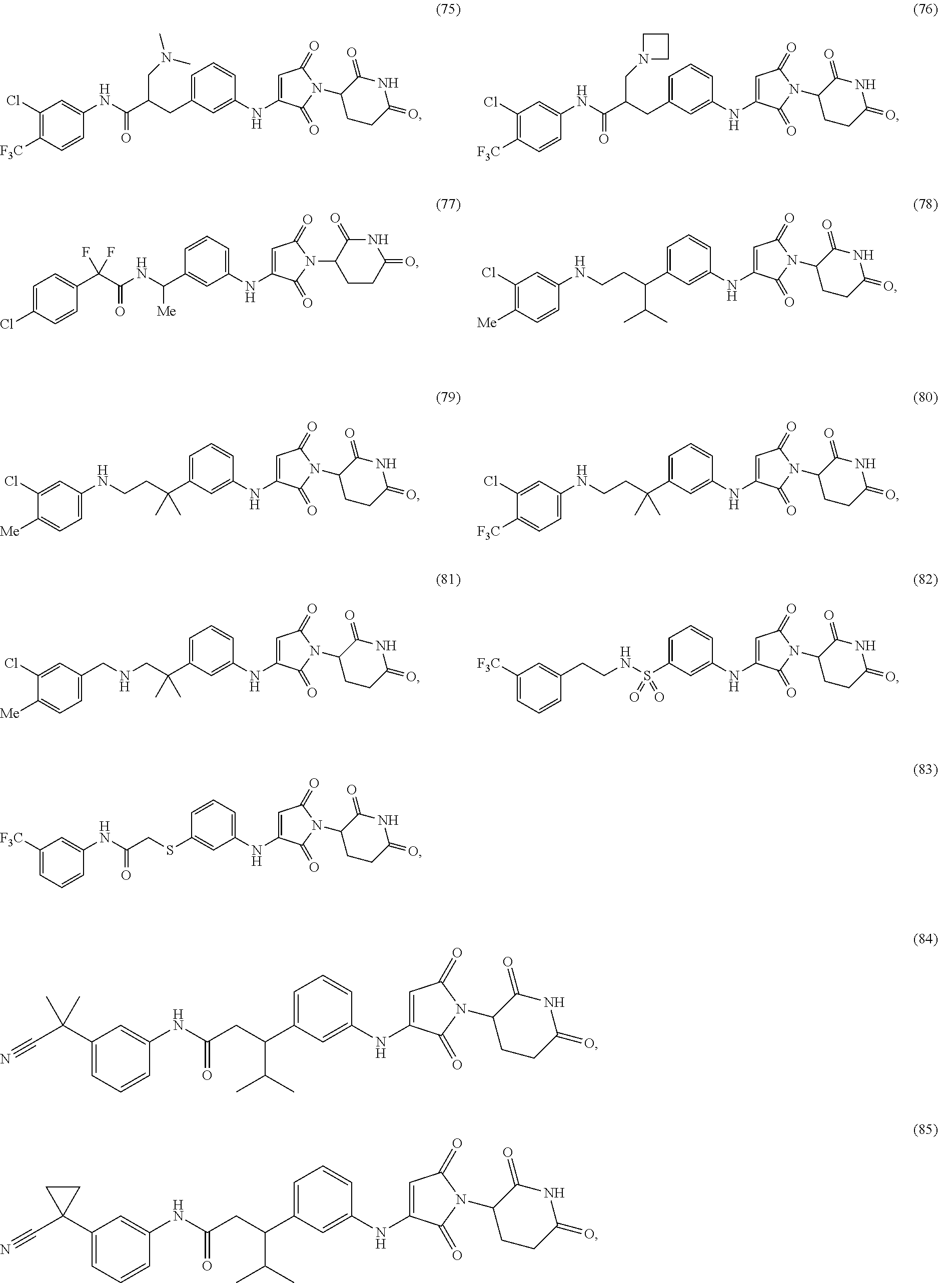 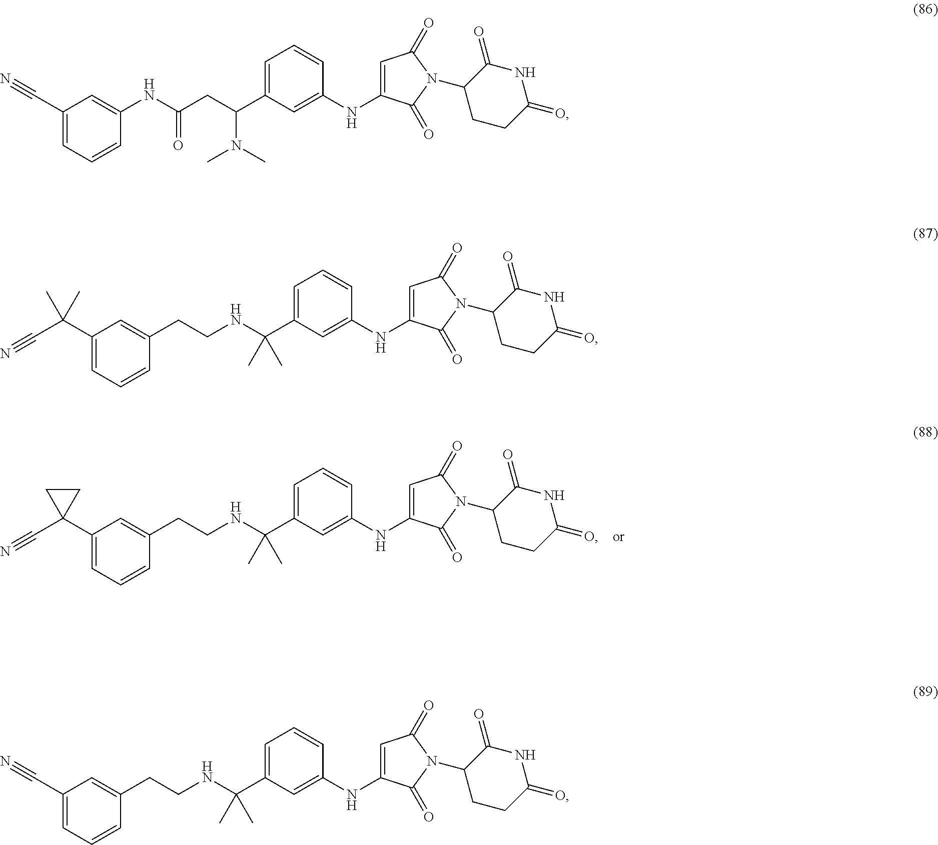 [SMILES: CN(C)C(CC(=O)Nc1cccc(C#N)c1)c1cccc(NC2=CC(=O)N(C3CCC(=O)NC3=O)C2=O)c1 ; CC(C)(C#N)c1cccc(CCNC(C)(C)c2cccc(NC3=CC(=O)N(C4CCC(=O)NC4=O)C3=O)c2)c1 ; CC(C)(NCCc1cccc(C2(C#N)CC2)c1)c1cccc(NC2=CC(=O)N(C3CCC(=O)NC3=O)C2=O)c1 ; CC(C)(NCCc1cccc(C#N)c1)c1cccc(NC2=CC(=O)N(C3CCC(=O)NC3=O)C2=O)c1]
or a pharmaceutically acceptable salt, hydrate, solvate, prodrug, stereoisomer, or tautomer thereof.

39. A pharmaceutical composition, comprising a therapeutically effective amount of the compound or pharmaceutically acceptable salt, hydrate, solvate, prodrug, stereoisomer, or tautomer of claim 1, and a pharmaceutically acceptable carrier.

40. A method of treating a disease or disorder that is characterized or mediated by activity of a protein that is a substrate for a complex between CRBN and the compound of claim 1, comprising administering to a subject in need thereof a therapeutically effective amount of the compound or pharmaceutically acceptable salt hydrate, solvate, prodrug, or tautomer or stereoisomer of claim 1.

41. The method of claim 40, wherein the disease or disorder is characterized or mediated by activity of IKZF1, IKZF2, or IKZF3.

42. The method of claim 41, wherein the disease or disorder is mediated by IKZF2 (Helios) activity.

43. The method of claim 42, wherein the disease or disorder is cancer.

44. The method of claim 43, wherein the cancer is T cell leukemia, T cell lymphoma, Hodgkin's lymphoma, non-Hodgkin's lymphoma, myeloid leukemia, non-small cell lung cancer (NSCLC), melanoma, triple-negative breast cancer (TNBC), nasopharyngeal cancer (NPC), microsatellite stable colorectal cancer (mssCRC), thymoma, carcinoid, or gastrointestinal stromal tumor (GIST).

45. The method of claim 40, wherein the disease or disorder is mediated by thioredoxin-interacting protein (TXNIP) activity.

46. The method of claim 45, wherein the disease or disorder is gout, idiopathic pulmonary fibrosis, silicosis, asbestosis, nonalcoholic steatohepatitis, atherosclerosis, diabetes, diabetic nephropathy, diabetic retinopathy, or diabetic cardiomyopathy.
